(19) United States
(12) Patent Application Publication
(10) Pub. No.: US 2015/0018369 A1
(43) Pub. Date: Jan. 15, 2015

(54) Compositions and Methods for Treating Malignant Astrocytomas

(71) Applicants: University of Washington through its Center for Commercialization, Seattle, WA (US); University of Montana, Missoula, MT (US)

(72) Inventors: Nephi Stella, Seattle, WA (US); Philippe Diaz, Missoula, MT (US)

(21) Appl. No.: 14/371,148

(22) PCT Filed: Jan. 9, 2013
(86) PCT No.: PCT/US13/20871

§ 371 Date: Jul. 8, 2014

Related U.S. Application Data
(60) Provisional application No. 61/584,808, filed on Jan. 9, 2012.

(51) Int. Cl. C07D 209/86; C07D 401/06; C07D 471/04

(52) U.S. Cl. CPC C07D 209/86; C07D 471/04; C07D 401/06
USPC 514/254.08; 548/441; 514/411; 546/86; 514/292; 546/165; 514/314; 544/372; 546/168; 546/85

(57) ABSTRACT

The disclosure provides methods of treating glioblastoma, methods of screening for compounds that treat glioblastoma, and pharmaceutical compositions useful in the treatment of glioblastoma.

### CROSS-REFERENCES TO RELATED APPLICATIONS

This application claims benefit of U.S. Provisional Application Ser. No. 61/584,808, filed Jan. 9, 2012, which is incorporated by reference herein in its entirety.

### STATEMENT REGARDING FEDERALLY SPONSORED RESEARCH

This invention was made with government support under NIH RO1 DA021285, DA014486, and P30-NS055022. The government has certain rights.

### BACKGROUND

1. Field of the Disclosure

The disclosure relates to methods and pharmaceutical compositions for treating brain tumors, and methods of screening for compounds that provide improved treatment of brain tumors.

2. Description of Related Art

There is an urgent need for novel therapeutics to treat brain tumors, especially astrocytomas grade IV (also known as glioblastomas multiforme). These tumors progress rapidely through healthy brain parenchyma and resist all current therapeutic approaches, making them one of the most devastating of all cancers. Patients diagnosed with astrocyomas grade IV typically die within a year. Even aggressive therapeutic interventions (i.e., combining gingery, radiotherapy and available chemotherapeutics) extend the life expectancy of these patients by only a few months. All drugs and adjuvants developed to kill astrocytomas (e.g., novel alkylating agents and monoclonal antibodies) have produced minimal therapeutic benefits. Thus, a radically different therapeutic approach needs to be identified and implemented so that we can reliably treat these devastating tumors.

Because of the shortcomings of existing brain cancer treatments, there is a need in the art for improved therapies that provide meaningful therapeutic intervention against brain tumors. In recent years, a number of studies have suggested the existence of receptors activated by the cannabinoid-like compounds: the alkylindoles (AI). There is a need in the art to identify new compounds that selectively activate AI receptors. AI receptors may also be implicated in disease and to use such receptors, alone or as part of a panel of other receptors, to identify and profile the effects of potential therapeutic compounds capable of treating one or other of the many diseases and disorders mediated by AI receptors.

### SUMMARY

The disclosure provides improved methods and pharmaceutical compositions for treating brain tumors. Also provided are methods of screening for compounds that provide improved treatment of brain tumors.

In broad aspect, the disclosure provides methods of treating or inhibiting cancer (e.g., glioblastoma), acognition disorder, schizophrenia, Alzheimer's disease and dementia, Parkinson's disease, depression, multiple sclerosis, amyotrophic lateral sclerosis (ALS), Huntington's disease, Fronto temporal dementia, parkinsonism linked to chromosome 17, and prion diseases, in a subject, the method comprising administering to the subject an effective amount of a compound of formula (I):

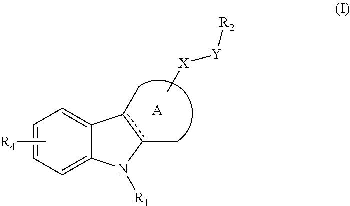

or a salt of prodrug of wherein:
ring A is a saturated or unsaturated 6 or 7-member ring, which can optionally contain one or more nitrogen atoms, and is optionally substituted with R₉;
X is selected from the group consisting of CH(OH), C═O, C═S, and S(O)₁₋₂;
Y is selected from the group consisting of absent, O, N(R₃), and C(R₃)(R₈);
R₁ is selected from the group consisting of optionally substituted alkyl, optionally substituted alkynyl, optionally substituted alkynyl, optionally substituted alkylcarbonyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₂ is selected from the group consisting of optionally substituted alkyl, optionally substituted alkynyl, optionally substituted alkynyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₃ is selected from the group consisting of optionally substituted alkyl, optionally substituted alkynyl, optionally substituted alkynyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical; or
or R₂ and R₃, with the atoms to which they are attached form an optionally substituted cycloalkyl, an optionally substituted heteroaryl, an optionally substituted heterocyclyl, or an optionally substituted aryl;
R₄ is selected from the group consisting of hydrogen, optionally substituted alkyl, optionally substituted alkynyl, optionally substituted alkynyl, optionally substituted cycloalkyl, optionally substituted aryl, optionally substituted aralkyl, optionally substituted heteroaryl, optionally substituted heterocyclyl, halogen, optionally substituted alkoxy, optionally substituted haloalkoxy, hydroxyl, N(R₅)(R₆), and polyether radical;
R₅ and R₆ are independently selected from the group consisting of hydrogen, optionally substituted alkyl, optionally substituted acyl, optionally substituted heteroalkyl, optionally substituted aryl, optionally substituted cycloalkyl, optionally substituted heteroaryl, and optionally substituted heterocyclyl;
R₈ is selected from the group consisting of hydrogen, optionally substituted alkyl, optionally substituted alkenyl, optionally substituted alkynyl, optionally substituted cycloalkyl, optionally substituted aryl, optionally substituted aralkyl, a halogen, optionally substituted alkoxy, and hydroxyl, or can form an optionally substituted cycloalkyl, an optionally substituted heteroaryl, an optionally substituted heterocyclyl, or an optionally substituted aryl with R₃; and R₉ is selected from the group consisting of hydrogen, optionally substituted alkyl, optionally substituted alkynyl, optionally substituted alkynyl, optionally substituted cycloalkyl, optionally substituted aryl, optionally substituted aralkyl, optionally substituted heteroaryl, optionally substituted heterocyclyl halogen, optionally substituted alkoxy, optionally substituted haloalkoxy, hydroxyl, and N(R₅)(R₆).

In broad aspect, the disclosure provides methods of treating or inhibiting canoes (e.g., glioblastoma), acognition disorder, schizophrenia, Alzheimer's disease and dementia, Parkinson's disease, depression, multiple sclerosis, amyotrophic lateral sclerosis (ALS), Huntington's disease, Fronto temporal dementia, parkinsonism linked to chromosome 17, and prion diseases, in a subject, the method comprising administering to the subject an effective amount of a compound of formula (II):

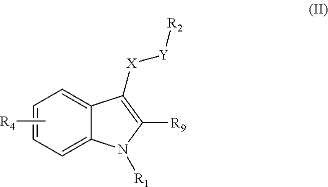

or a salt of prodrug of wherein:
X is selected from the group consisting of CH(OH), C═O, C═S, and S(O)₁₋₂;
Y is selected from the group consisting of absent, O, N(R₃), and C(R₃)(R₈);
R₁ is selected from the group consisting of optionally substituted alkyl, optimally substituted alkynyl, optionally substituted alkynyl, optionally substituted alkylcarbonyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₂ is selected from the group consisting of optionally substituted alkyl, optionally substituted alkynyl, optionally substituted alkynyl, optionally substituted haloalkyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₃ is selected from the group consisting of optionally substituted alkyl, optionally substituted alkynyl, optionally substituted alkynyl, optionally substituted cycloalkyl, optionally substituted heteroaryl, optionally substituted heterocyclyl, optionally substituted aryl, optionally substituted aralkyl, optionally substituted heteroaralkyl, optionally subs-tinged (heterocyclyl)alkyl, and polyether radical; or
or R₂ and R₃, with the atoms to which they are attached form an optionally substituted cycloalkyl, an optionally substituted heteroaryl, an optionally substituted heterocyclyl, Of an optionally substituted aryl;
R₄ is selected from the group consisting of hydrogen, optionally substituted alkyl, optionally substituted alkynyl, optionally substituted alkynyl, optionally substituted cycloalkyl, optionally substituted awl, optionally substituted aralkyl, optionally substituted heteroaryl, optionally substituted heterocyclyl, halogen, optionally substituted alkoxy, optionally substituted haloalkoxy, hydroxyl, N(R₅)(R₆), and polyether radical;
R₅ and R₆ are independently selected from the group consisting of hydrogen, optionally substituted alkyl, optionally substituted acyl, optionally substituted heteroalkyl, optionally substituted aryl, optionally substituted cycloalkyl, optionally substituted heteroaryl, and optionally substituted heterocyclyl;
R₈ is selected from the group consisting of hydrogen, optionally substituted alkyl, optionally substituted alkenyl, optionally substituted alkynyl, optionally substituted cycloalkyl, optionally substituted aryl, optionally substituted aralkyl, a halogen, optionally substituted alkoxy, and hydroxyl, or can form an optionally substituted cycloalkyl, an optionally substituted heteroaryl, an optionally substituted heterocyclyl, or an optionally substituted aryl with R₃; and
R₉ is selected from the group consisting of hydrogen, optionally substituted alkyl, optionally substituted alkynyl, optionally substituted alkynyl, optionally substituted cycloalkyl, optionally substituted aryl, optionally substituted aralkyl, optionally substituted heteroaryl, optionally substituted heterocyclyl, halogen, optionally substituted alkoxy, optionally substituted haloalkoxy, hydroxyl, and N(R₅)(R₆).

In broad aspect, the disclosure provides methods of treating or inhibiting cancer (e.g., glioblastoma), acognition disorder, schizophrenia, Alzheimer's disease and dementia, Parkinson's disease, depression, multiple sclerosis, amyotrophic lateral sclerosis (ALS), Huntington's disease, Pronto temporal dementia, parkinsonism linked to chromosome 17, and prion diseases, in a subject, the method comprising administering to the subject an effective amount of a compound of formula (IV):

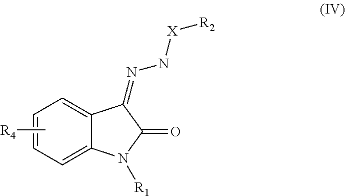

or a salt of prodrug of wherein:
X is C═O;
R₁ is selected from the group consisting of optionally substituted alkyl, optionally substituted alkynyl, optionally substituted alkynyl, optionally substituted alkylcarbonyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₂ is selected from the group consisting of optionally substituted alkyl, optionally substituted alkynyl, optionally substituted alkynyl, optionally substituted haloalkyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₄ is selected from the group consisting of hydrogen, optionally substituted alkyl, optionally substituted alkynyl, optionally substituted alkynyl, optionally substituted cycloalkyl, optionally substituted aryl, optionally substituted aralkyl, optionally substituted heteroaryl, optionally substituted heterocyclyl, halogen, optionally substituted alkoxy, optionally substituted haloalkoxy, hydroxyl, N(R₅)(R₆), and polyether radical; and
R₅ and R₆ are independently selected from the group consisting of hydrogen, optionally substituted alkyl, optionally substituted acyl, optionally substituted heteroalkyl, optionally substituted aryl, optionally substituted cycloalkyl, optionally substituted heteroaryl, and optionally substituted heterocyclyl.

In one aspect, the disclosure provides for methods of activating the GPR124 receptor comprising administering a compound formula (I) or formula (II) or formula (IV).

In another aspect, the disclosure provides for a method of screening for therapeutic agents useful in the treatment of glioblastomas.

The disclosure also provides compounds of formula (I)

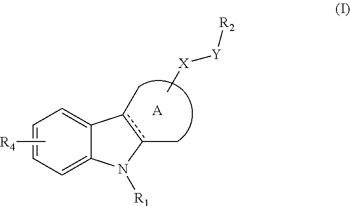

or a salt of prodrug of wherein:
ring A is a saturated or unsaturated 6 or 7-member ring, which can optionally contain one or more nitrogen atoms, and is optionally substituted with R₉;
X is selected from the group consisting of CH(OH), C═O, C═S, and S(O)₁₋₂;
Y is selected from the group consisting of absent, O, N(R₃), and C(R₃)(R₈);
R₁ is selected from the group consisting of optionally substituted alkyl, optionally substituted alkynyl, optionally substituted alkynyl, optionally substituted alkylcarbonyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₂ is selected from the group consisting of optionally substituted alkyl, optionally substituted alkynyl, optionally substituted alkynyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₃ is selected from the group consisting of optionally substituted alkyl, optionally substituted alkynyl, optionally substituted alkynyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical; or
or R₂ and R₃, with the atoms to which they are attached form an optionally substituted cycloalkyl, an optionally substituted heteroaryl, an optionally substituted heterocyclyl, or an optionally substituted aryl;
R₄ is selected from the group consisting of hydrogen, optionally substituted alkyl, optionally substituted alkynyl, optionally substituted alkynyl, optionally substituted cycloalkyl, optionally substituted aryl, optionally substituted aralkyl, optionally substituted heteroaryl, optionally substituted heterocyclyl, halogen, optionally substituted alkoxy, optionally substituted haloalkoxy, hydroxyl, N(R₅)(R₆), and polyether radical;
R₅ and R₆ are independently selected from the group consisting of hydrogen, optionally substituted alkyl, optionally substituted acyl, optionally substituted heteroalkyl, optionally substituted aryl, optionally substituted cycloalkyl, optionally substituted heteroaryl, and optionally substituted heterocyclyl;
R₈ is selected from the group consisting of hydrogen, optionally substituted alkyl, optionally substituted alkenyl, optionally substituted alkynyl, optionally substituted cycloalkyl, optionally substituted aryl, optionally substituted aralkyl, a halogen, optionally substituted alkoxy, and hydroxyl, or can form an optionally substituted cycloalkyl, an optionally substituted heteroaryl, an optionally substituted heterocyclyl, or an optionally substituted aryl with R₃; and R₉ is selected from the group consisting of hydrogen, optionally substituted alkyl, optionally substituted alkynyl, optionally substituted alkynyl, optionally substituted cycloalkyl, optionally substituted aryl, optionally substituted aralkyl, optionally substituted heteroaryl, optionally substituted heterocyclyl, halogen, optionally substituted alkoxy, optionally substituted haloalkoxy, hydroxyl, and N(R₅)(R₆).

The disclosure further provides compounds of formula (II)

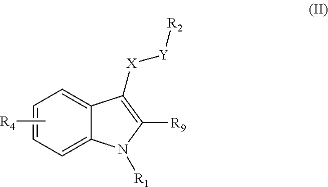

or a salt of prodrug of wherein:
X is selected from the group consisting of CH(OH), C═O, C═S, and S(O)₁₋₂;
Y is selected from the group consisting of absent, O, N(R₃), and C(R₃)(R₈);
R₁ is selected from the group consisting of optionally substituted alkyl, optionally substituted alkynyl, optionally substituted alkynyl, optionally substituted alkylcarbonyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₂ is selected from the group consisting of optionally substituted alkyl, optionally substituted alkynyl, optionally substituted alkynyl, optionally substituted haloalkyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₃ is selected from the group consisting of optionally substituted alkyl, optionally substituted alkynyl, optionally substituted alkynyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical; or
or R₂ and R₃, with the atoms to which they are attached form an optionally substituted cycloalkyl, an optionally substituted heteroaryl, an optionally substituted heterocyclyl or an optionally substituted aryl;
R₄ is selected from the group consisting of hydrogen, optionally substituted alkyl, optionally substituted alkynyl, optionally substituted alkynyl, optionally substituted cycloalkyl, optionally substituted aryl, optionally substituted aralkyl, optionally substituted heteroaryl, optionally substituted heterocyclyl, halogen, optionally substituted alkoxy, optionally substituted haloalkoxy, hydroxyl, N(R₅)(R₆), and polyether radical;
R₅ and R₆ are independently selected from the group consisting of hydrogen, optionally substituted alkyl, optionally substituted acyl, optionally substituted heteroalkyl, optionally substituted aryl, optionally substituted cycloalkyl, optionally substituted heteroaryl, and optionally substituted heterocyclyl;
R₈ is selected from the group consisting of hydrogen, optionally substituted alkyl, optionally substituted alkenyl, optionally substituted alkynyl, optionally substituted cycloalkyl, optionally substituted aryl, optionally substituted aralkyl, a halogen, optionally substituted alkoxy, and hydroxyl, or can form an optionally substituted cycloalkyl, an optionally substituted heteroaryl, an optionally substituted heterocyclyl, or an optionally substituted aryl with R₃; and
R₉ is selected from the group consisting of hydrogen, optionally substituted alkyl, optionally substituted alkynyl, optionally substituted alkynyl, optionally substituted cycloalkyl, optionally substituted aryl, optionally substituted aralkyl, optionally substituted heteroaryl, optionally substituted heterocyclyl, halogen, optionally substituted alkoxy, optionally substituted haloalkoxy, hydroxyl, and N(R₅)(R₆);
provided the compound is not

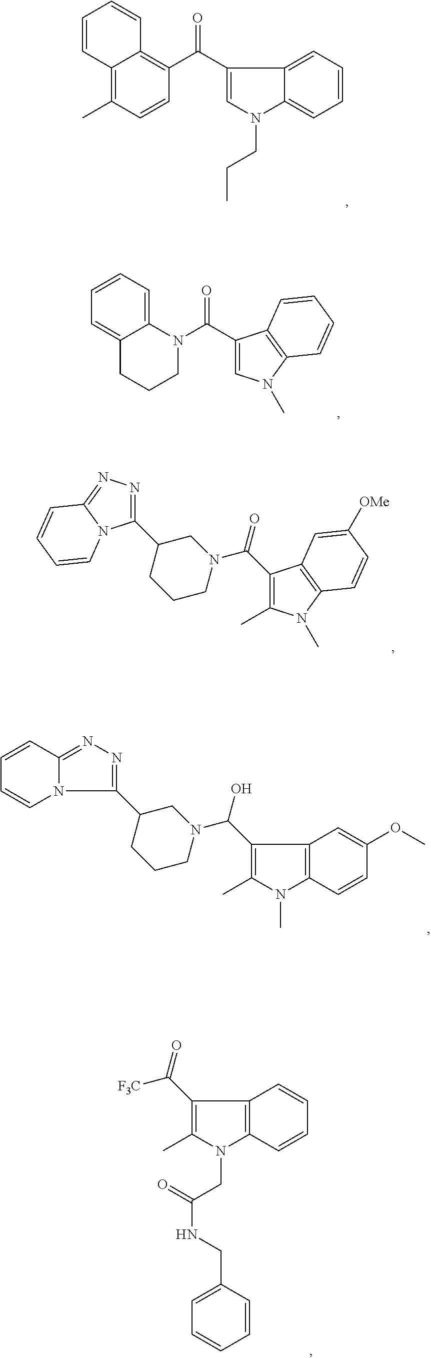 [SMILES: CCCn1cc(C(=O)c2ccc(C)c3ccccc23)c2ccccc21 ; Cn1cc(C(=O)N2CCCc3ccccc32)c2ccccc21 ; COc1ccc2c(c1)c(C(=O)N1CCCC(c3nnc4ccccn34)C1)c(C)n2C ; COc1ccc2c(c1)c(C(O)N1CCCC(c3nnc4ccccn34)C1)c(C)n2C ; Cc1c(C(=O)C(F)(F)F)c2ccccc2n1CC(=O)NCc1ccccc1]

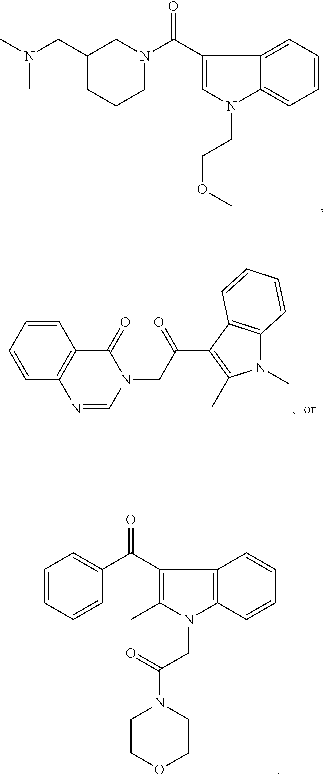 [SMILES: COCCn1cc(C(=O)N2CCCC(CN(C)C)C2)c2ccccc21 ; Cc1c(C(=O)Cn2cnc3ccccc3c2=O)c2ccccc2n1C ; Cc1c(C(=O)c2ccccc2)c2ccccc2n1CC(=O)N1CCOCC1]

The disclosure also provides compounds of formula (IV)

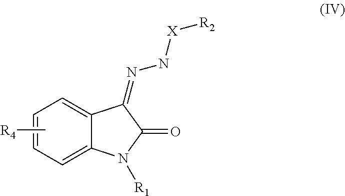

or a salt of prodrug of wherein:
X is C═O;
R₁ is selected from the group consisting of optionally substituted alkyl, optionally substituted alkynyl, optionally substituted alkynyl, optionally substituted alkylcarbonyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₂ is selected from the group consisting of optionally substituted alkyl, optionally substituted alkynyl, optionally substituted alkynyl, optionally substituted haloalkyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₄ is selected from the group consisting of hydrogen, optionally substituted alkyl, optionally substituted alkynyl, optionally substituted alkynyl, optionally substituted cycloalkyl, optionally substituted aryl, optionally substituted aralkyl, optionally substituted heteroaryl, optionally substituted heterocyclyl, halogen, optionally substituted alkoxy, optionally substituted haloalkoxy, hydroxyl, N(R₅)(R₆), and polyether radical; and
R₅ and R₆ are independently selected from the group consisting of hydrogen, optionally substituted alkyl, optionally substituted acyl, optionally substituted heteroalkyl, optionally substituted aryl, optionally substituted cycloalkyl, optionally substituted heteroaryl, and optionally substituted heterocyclyl.

The disclosure also provides compounds that are:

| Name | Structure |
|---|---|
| (4-methylnaphthalen-1-yl)(1-(2- morpholinoethyl)-2-(trifluoromethyl)- 1H-indol-3-yl)methanone | 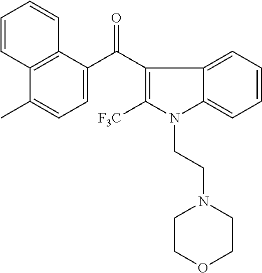 [SMILES: Cc1ccc(C(=O)c2c(C(F)(F)F)n(CCN3CCOCC3)c3ccccc23)c2ccccc12] |
| (2-methyl-1-(2,2,2-trifluoroethyl)-1H- indol-3-yl)(4-methylnaphthalen-1- yl)methanone | 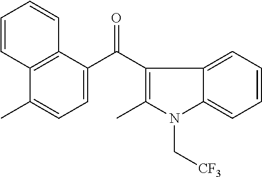 [SMILES: Cc1ccc(C(=O)c2c(C)n(CC(F)(F)F)c3ccccc23)c2ccccc12] |
| (1-cyclopropyl-2-methyl-1H-indol-3- yl)(4-methylnaphthalen-1- yl)methanone | 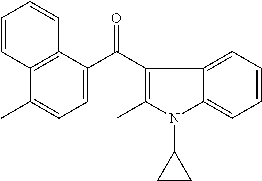 [SMILES: Cc1ccc(C(=O)c2c(C)n(C3CC3)c3ccccc23)c2ccccc12] |
| (2-methyl-1-(2-morpholinoethyl)-1H- pyrrolo[2,3-c]pyridin-3-yl)(4- methylnaphthalen-1-yl)methanone | 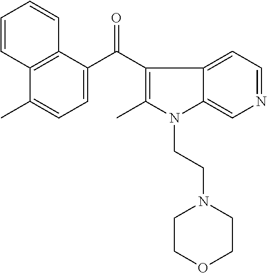 [SMILES: Cc1ccc(C(=O)c2c(C)n(CCN3CCOCC3)c3cnccc23)c2ccccc12] |
| (9-ethyl-9H-carbazol-3-yl)(4- methylnaphthalen-1-yl)methanone | 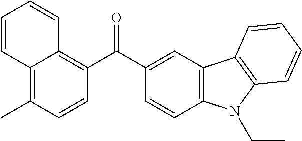 [SMILES: CCn1c2ccccc2c2cc(C(=O)c3ccc(C)c4ccccc34)ccc21] |
| (Z)-N′-(1-ethyl-2-oxoindolin-3- ylidene)-1-naphthohydrazide | 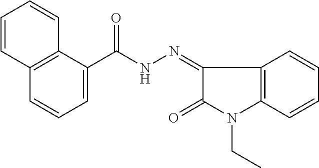 [SMILES: CCN1C(=O)/C(=N\NC(=O)c2cccc3ccccc23)c2ccccc21] |
| (1-ethyl-2-methyl-1H-indol-3-yl)(4- methoxyphenyl)methanone | 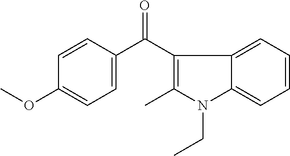 [SMILES: CCn1c(C)c(C(=O)c2ccc(OC)cc2)c2ccccc21] |
| (3,4-dihydroquinolin-1(2H)-yl)(9- ethyl-9H-carbazol-3-yl)methanone | 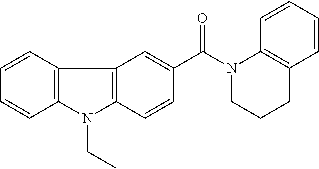 [SMILES: CCn1c2ccccc2c2cc(C(=O)N3CCCc4ccccc43)ccc21] |
| ethyl 5-ethyl-8-(4-methyl-1- naphthoyl)-3,4-dihydro-1H-pyrido[4,3- b]indole-2(5H)-carboxylate | 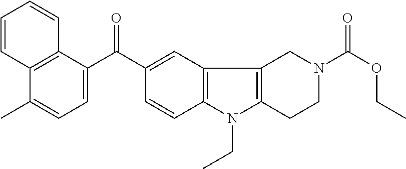 [SMILES: CCOC(=O)N1CCc2c(c3cc(C(=O)c4ccc(C)c5ccccc45)ccc3n2CC)C1] |
| (4-methylnaphthalen-1-yl)(9-propyl- 9H-carbazol-3-yl)methanone | 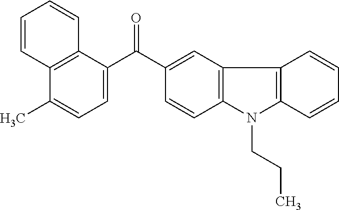 [SMILES: CCCn1c2ccccc2c2cc(C(=O)c3ccc(C)c4ccccc34)ccc21] |
| 2-(7-Fluorobenzofurazan-4-sulfonoyl)- 5-ethyl-1H,2H,3H,4H,5H-pyrido[4,3- b]indole | 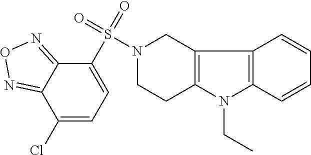 [SMILES: CCn1c2c(c3ccccc31)CN(S(=O)(=O)c1ccc(Cl)c3nonc13)CC2] |
| (5-ethyl-3,4-dihydro-1H-pyrido[4,3- b]indol-2(5H)-yl)(4-methylnaphthalen- 1-yl)methanone | 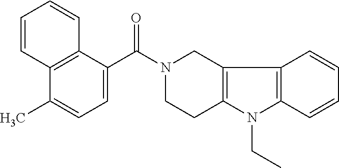 [SMILES: CCn1c2c(c3ccccc31)CN(C(=O)c1ccc(C)c3ccccc13)CC2] |
| (9-ethyl-9H-carbazol-3-yl)(4- methylpiperazin-1-yl)methanone | 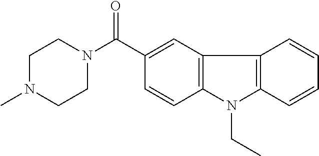 [SMILES: CCn1c2ccccc2c2cc(C(=O)N3CCN(C)CC3)ccc21] |
| (9-ethyl-9H-carbazol-3-yl)(p- tolyl)methanone | 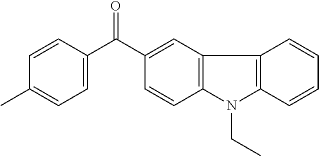 [SMILES: CCn1c2ccccc2c2cc(C(=O)c3ccc(C)cc3)ccc21] |
| (9H-carbazol-9-yl)(4- methylnaphthalen-1-yl)methanone | 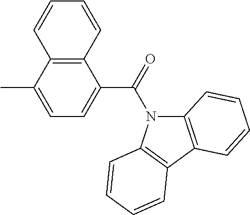 [SMILES: Cc1ccc(C(=O)n2c3ccccc3c3ccccc32)c2ccccc12] |
| (9-ethyl-9H-carbazo1-3-yl)(quinolin-8- yl)methanone | 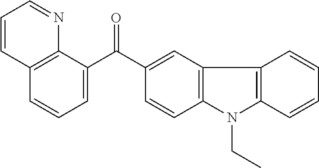 [SMILES: CCn1c2ccccc2c2cc(C(=O)c3cccc4cccnc34)ccc21] |
| (9-ethyl-9H-carbazol-3-yl)(quinolin-5-N yl)methanone | 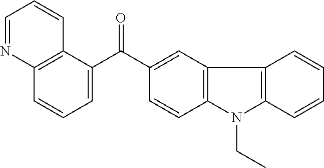 [SMILES: CCn1c2ccccc2c2cc(C(=O)c3cccc4ncccc34)ccc21] |
| (9-ethyl-9H-carbazol-3-yl)(naphthalen- 1-yl)methanone | 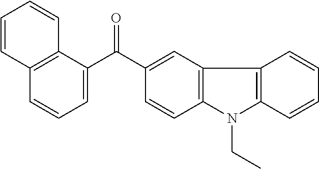 [SMILES: CCn1c2ccccc2c2cc(C(=O)c3cccc4ccccc34)ccc21] |
| 1-(9-ethyl-9H-carbazol-3-yl)-2- phenylethanone | 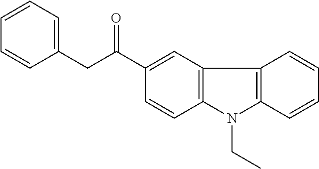 [SMILES: CCn1c2ccccc2c2cc(C(=O)Cc3ccccc3)ccc21] |
| (9-ethyl-9H-carbazol-3-yl)(1,2,3,4- tetrahydroquinolin-8-yl)methanone | 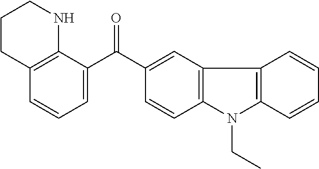 [SMILES: CCn1c2ccccc2c2cc(C(=O)c3cccc4c3NCCC4)ccc21] |
| (9-ethyl-9H-carbazo1-3-yl)(1,2,3,4- tetrahydroquinolin-5-yl)methanone | 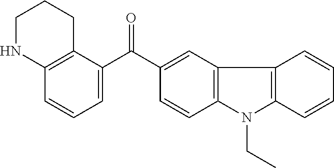 [SMILES: CCn1c2ccccc2c2cc(C(=O)c3cccc4c3CCCN4)ccc21] |

The disclosure also provides a pharmaceutical composition comprising a therapeutically effective amount of a compound of formula (I) or formula (II) or formula (IV), and one or more pharmaceutically acceptable diluents, preservatives, solubilizers, emulsifiers, adjuvants, excipients, or carriers.

The disclosure also provides methods of preparing compounds of the disclosure and the intermediates used in those methods.

The disclosure further provides a compound or pharmaceutical composition of the disclosure thereof in a kit with instructions for using the compound or composition.

The disclosure further provides compounds of the disclosure that may be administered alone or in combination with other drugs or therapies known to be effective to treat the disease to enhance overall effectiveness of therapy.

### BRIEF DESCRIPTION OF THE DRAWINGS

FIG. 1 illustrates toxic profile of a) THC, b) CP55940, c) WIN 55212-2 (ST-1) and d) ST-11 in sknmc, MDA231 and T98 g cells. Indicated are the EC₅₀ of the respective toxic effect.

FIG. 2 shows the effect of ST-11, ST-23, ST-25, ST-29 and ST-48 on human CB₁ and CB₂ receptors. ST-11 competes for [³H]-WIN55212-2 binding in HEK cells (circles), and not for [³H]-CP5540 at CB₁ (triangles) and CB₂ (squares).

FIG. 3 shows the potency of standard care therapeutics (BCNU and temozolamide, TMZ) compare to ST-11, ST-25 and ST-34 when tested in human T98 g cells and HepG2 cells, providing the in vitro therapeutic index of these compounds.

FIG. 4 shows the potency of ST-11, ST-25 and ST-34 in human astrocytomas cell lines and astrocytoma cells derived from patients.

FIG. 5 shows siRNA identification (following the decline of mRNA over 4 days in vitro to ascertain for knockdown stability). Sequence homology between CB₂ receptors and GPR124 within the third transmembrane domain, which contains an interaction site for alkylindole binding to CB₂ receptors.

FIG. 6 shows that HEK293 cells do not express CB₁ and CB₂ receptors as measured by radioligand binding, but express AI receptors as indicated by abundant [³H]-WIN55212-2 binding and activation of GTP□S binding and inhibition of cAMP production by ST-11, suggesting that AI receptor couple to Gifu proteins.

FIG. 7 shows that CBX-003, CBX-005 and CBX-009 activate GTP□S binding in HEK293 cell homogenates, indicating that these compounds act as agonists at AI receptors.

FIG. 8 shows that in T98 g cells in culture ST-11 induces the activation of polo-like kinase 1 (PLK-1), promotes the cleavage of PARP (a) and activates caspase 3 (b) within hours, which is followed by cell death as measured by reduction in cell number (c), nuclear fragmentation (d) and cell blebbing after 48 hrs.

FIG. 9 shows that DBT cells, a mouse astrocytoma cell line, does not express CB₁ and CB₂ receptors as measured by radioligand binding (a), yet likely express AI receptors as measured by radioligand binding competition with ST compounds.

FIG. 10 illustrates that ST-compounds do not stipulate cell migration (a), yet inhibit DBT cell migration stimulated by LPA (b). ST-11 kills DBT cells but not mouse neurons in primary culture (c).

FIG. 11 shows that ST-compounds, similarly to the chemoattactant LPA, increase the number of focal adhesion in the human astrocytoma cell line U87MG cells.

FIG. 12 shows that mouse microglia in primary culture express AI receptors as suggested by [³H]-WIN55212 binding competed by ST-11 (a). ST-11 inhibits cAMP production stimulated by Isoproterenol (b) and microglia cell migration stimulated by ATP (c). ST-11 does not stimulate or inhibit IP production (d and e), NO production (f) and affect cell viability in microglia.

FIG. 13 illustrates an LC-MS chromatogram and calibration curve of ST-11 (a & b). PK profile of ST-11 (c, d, e & f). ST-11 does not influence locomotor activity on an accelerating rotarod, suggesting lack of acute toxicity.

FIG. 14 shows that ST-11 increases the number of lymphocytes (a, b) and microglia (c, d) in DBT tumors implanted in BalbC mice and treated daily over 2 weeks.

FIG. 15 shows the effect of ST-11 on mouse microglia (a) and lymphocyte cell number (c), as well as on cell division (b) and overall tumor volume (d) in 3 week DBT tumors implanted in BalbC mice brain and treated daily with ST-11 (i.p.) over 2 weeks.

FIG. 16 shows that ST-compounds compete for [³H]-WIN55212-2 binding in human sknmc cells, suggesting that these cells express AI receptors.

FIG. 17 shows that ST-compounds compete for [³H]-WIN55212-2 binding in CB₁^{-/-} mouse brain homogenates, suggesting that neurons express AI receptors.

FIG. 18 illustrates the potency of ST compounds at killing human melanoma cells lines in culture.

### DETAILED DESCRIPTION

Briefly stated, the disclosure provides compounds, pharmaceutical compositions, and methods for treating brain tumors (e.g., glioblastoma) in a subject. Also provided are methods of treating cancer, acognition disorder, schizophrenia, Alzheimer's disease and dementia, Parkinson's disease, depression, multiple sclerosis, amyotrophic lateral sclerosis (ALS), Huntington's disease, Fronto temporal dementia, parkinsonism linked to chromosome 17, and prion diseases, in a subject. Also provided are methods of screening for compounds and adjuvants that provide improved treatment of brain tumors.

The disclosure provides compounds of formula (I), which are of formula (III):

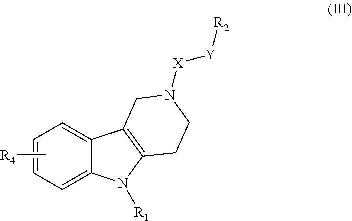

or a salt of prodrug of wherein:
X is selected from the group consisting of C═O, C═S, and S(O)₁₋₂;
Y is selected from the group consisting of absent, O, N(R₃), and C(R₃)(R₈);
R₁ is selected from the group consisting of optionally substituted alkyl, optionally substituted alkynyl, optionally substituted alkynyl, optionally substituted alkylcarbonyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₂ is selected from the group consisting of optionally substituted alkyl, optionally substituted alkynyl, optionally substituted alkynyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₃ is selected from the group consisting of optionally substituted alkyl, optionally substituted alkynyl, optionally substituted alkynyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical; or
or R₂ and R₃, with the atoms to which they are attached form an optionally substituted cycloalkyl, an optionally substituted heteroaryl, an optionally substituted heterocyclyl, or an optionally substituted aryl;
R₄ is selected from the group consisting of hydrogen, optionally substituted alkyl, optionally substituted alkynyl, optionally substituted alkynyl, optionally substituted cycloalkyl, optionally substituted aryl, optionally substituted aralkyl, optionally substituted heteroaryl, optionally substituted heterocyclyl, halogen, optionally substituted alkoxy, optionally substituted haloalkoxy, hydroxyl, N(R₅)(R₆), and polyether radical;
R₅ and R₆ are independently selected from the group consisting of hydrogen, optionally substituted alkyl, optionally substituted acyl, optionally substituted heteroalkyl, optionally substituted aryl, optionally substituted cycloalkyl, optionally substituted heteroaryl, and optionally substituted heterocyclyl; and
R₈ is selected from the group consisting of hydrogen, optionally substituted alkyl, optionally substituted alkenyl, optionally substituted alkynyl, optionally substituted cycloalkyl, optionally substituted aryl, optionally substituted aralkyl, a halogen, optionally substituted alkoxy, and hydroxyl, or can form an optionally substituted cycloalkyl, an optionally substituted heteroaryl, an optionally substituted heterocyclyl, of an optionally substituted aryl with R₃.

In one embodiment, the disclosure provides compounds of formula (II) that are of formula (IIa):

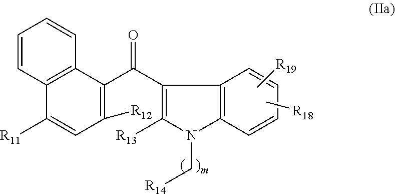

or pharmaceutically acceptable salts, wherein
R₁₁ is C₁-C₆ alkyl, C₂-C₆ alkenyl, C₂-C₆ alkynyl, C₁-C₆ haloalkyl, C₁-C₆ alkoxy, C₁-C₆ hydroxyalkyl, C₁-C₆ haloalkoxy, halogen, —NO₂, —CN, —OR₁₅, —SR₁₅, —C(O)R₁₅, —NHC(O)R₁₅, —C(O)OR₁₅, —OC(O)R₁₅, —NR₁₆R₁₇, —C(O)NR₁₆R₁₇, —NHR₁₅C(O)NR₁₆R₁₇, —SO₂NR₁₆R₁₇, alkylaryl, cycloalkyl, heterocyclyl, or heteroaryl, each being optionally substituted with one to four R₂₀;
R₁₂ is H, C₁-C₆ alkyl, C₂-C₆ alkenyl, C₂-C₆ alkynyl, C₁-C₆ haloalkyl, C₁-C₆ alkoxy, C₁-C₆ hydroxyalkyl, C₁-C₆ haloalkoxy, halogen, —NO₂, —CN, —OR₁₅, —SR₁₅, —C(O)R₁₅, —NHC(O)R₁₅, —C(O)OR₁₅, —OC(O)R₁₅, —NR₁₆R₁₇, —C(O)NR₁₆R₁₇, —NHR₁₅C(C)NR₁₆R₁₇, —SO₂NR₁₆R₁₇, alkylaryl, cycloalkyl, heterocyclyl, or heteroaryl, each being optionally substituted with one to four R₂₀;
R₁₃ is C₁-C₆ alkyl, C₂-C₆ alkenyl, C₂-C₆ alkynyl, C₁-C₆ haloalkyl, C₁-C₆ alkoxy, C₁-C₆ hydroxyalkyl, C₁-C₆ haloalkoxy, halogen, —NO₂, —CN, —OR₁₅, —SR₁₅, —C(O)R₁₅, —NHC(O)R₁₅, —C(O)OR₁₅, —C(O)OR₁₅, —OC(O)R₁₅, —NR₁₆R₁₇, —C(O)NR₁₆R₁₇, —NHR₁₅C(O)NR₁₆R₁₇, —SO₂NR₁₆R₁₇, alkylaryl, cycloalkyl, heterocyclyl, or heteroaryl, each being optionally substituted with one to four R₂₀;
R₁₄ is H, C₁-C₆ alkyl, C₂-C₆ alkenyl, C₂-C₆ alkynyl, aryl, C₁-C₆ alkylaryl, cycloalkyl, heterocyclyl, or heteroaryl, each being optionally substituted with one to foot R₂₀, or
R₁₄ and R₁₃ taken together with the atoms to which they are attached form a 5-, 6-, or 7-membered heterocyclyl group optionally substituted with one to four R₂₀;
R₁₅, R₁₆, and R₁₇ are independently H, C₁-C₆ alkyl, C₂-C₆ alkenyl C₂-C₆ alkenyl, C₁-C₆ alkoxy, halogen, hydroxyl, aryl, alkylaryl, cycloalkyl, heterocyclyl, or heteroaryl, each being optionally substituted with one to four R₂₀;
R₁₈ and R₁₉ are each independently H, C₁-C₆ alkyl, C₂-C₆ alkenyl, C₂-C₆ alkynyl, C₁-C₆ haloalkyl, C₁-C₆ alkoxy, C₁-C₆ haloalkoxy, C₁-C₆ hydroxyalkyl, halogen, —NO₂, —CN, —OR₁₅, —SR₁₅, —C(O)R₁₅, —NHC(O)R₁₅, —C(O)OR₁₅, —OC(O)R₁₅, —NR₁₆R₁₇, —C(C)NR₁₆R₁₇, —NHR₁₅C(O)NR₁₆R₁₇, —SO₂NR₁₆R₁₇, alkylaryl, cycloalkyl, heterocyclyl, or heteroaryl, each being optionally substituted with one to four R₂₀,
R₂₀ is halogen, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxy, C₁-C₆ haloalkyl, C₁-C₆ alkoxy, C₁-C₆ haloalkoxy, —CO₂H, —CO₂(C₁-C₆ alkyl), —SO₂(C₁-C₆ alkyl), —CONH₂, —CONH(C₁-C₆ alkyl), —CON(C₁-C₆ alkyl)₂, —CON(H)OH, —NHCO(C₁-C₆ alkyl), or —NHCO₂(C₁-C₆ alkyl); and
m is an integer 1 or 2.

In another embodiment, the disclosure provides compounds of formula (IIa) wherein:
R₁₁ is C₁-C₆ alkyl, C₂-C₆ alkenyl, C₂-C₆ alkynyl, C₁-C₆ haloalkyl, C₁-C₆ alkoxy, C₁-C₆ hydroxyalkyl, C₁-C₆ haloalkoxy, halogen, —NO₂, —CN, —OR₁₅, —SR₁₅, —C(O)R₁₅, —NHC(O)R₁₅, —C(O)OR₁₅, —OC(O)R₁₅, —NR₁₆R₁₇, —C(O)NR₁₆R₁₇, —NHR₁₅C(O)NR₁₆R₁₇, —SO₂NR₁₆R₁₇, alkylaryl, cycloalkyl, heterocyclyl, or heteroaryl, each being optionally substituted with one to four R₂₀;
R₁₂ is H, C₁-C₆ alkyl, C₂-C₆ alkenyl, C₂-C₆ alkynyl, C₁-C₆ haloalkyl, C₁-C₆ alkoxy C₁-C₆ hydroxyalkyl, C₁-C₆ haloalkoxy, halogen, —NO₂, —CN, —OR₁₅, —SR₁₅, —C(O)R₁₅, —NHC(O)R₁₅, —C(O)OR₁₅, —OC(O)R₁₅, NR₁₆R₁₇, —C(O)NR₁₆R₁₇, —NHR₁₅C(O)NR₁₆R₁₇, —SO₂NR₁₆R₁₇, alkylaryl, cycloalkyl, heterocyclyl, or heteroaryl, each being optionally substituted with one to four R₂₀;
R₁₃ is C₁-C₆ alkyl, C₂-C₆ alkenyl, C₂-C₆ alkynyl, C₁-C₆ haloalkyl, C₁-C₆ alkoxy, C₁-C₆ hydroxyalkyl, C₁-C₆ haloalkoxy, halogen, —NO₂, —CN, —OR₁₅, —SR₁₅, —C(O)R₁₅, —NHC(O)R₁₅, —C(O)OR₁₅, —OC(O)R₁₅, —NR₁₆R₁₇, —C(O)NR₁₆R₁₇, —NHR₁₅C(O)NR₁₆R₁₇, —SO₂NR₁₆R₁₇, alkylaryl, cycloalkyl, heterocyclyl, or heteroaryl, each being optionally substituted with one to four R₂₀;
R₁₄ is H, C₁-C₆ alkyl, C₂-C₆ alkenyl, C₂-C₆ alkynyl, aryl, C₁-C₆ alkylaryl, cycloalkyl, heterocyclyl, or heteroaryl, each being optionally substituted with one to four R₂₀, or
R₁₄ and R₁₃ taken together with the atoms to which they are attached form a 5-, 6-, or 7-membered heterocyclyl group optionally substituted with one to four R₂₀;
R₁₅, R₁₆, and R₁₇ are independently H, C₁-C₆ alkyl, C₂-C₆ alkenyl C₂-C₆ alkynyl, C₁-C₆ alkoxy, halogen, hydroxyl, aryl, alkylaryl, cycloalkyl, heterocyclyl, or heteroaryl, each being optionally substituted with one to four R₂₀;
R₁₈ and R₁₉ are each independently H, C₁-C₆ alkyl, C₂-C₆ alkenyl, C₂-C₆ alkynyl, C₁-C₆ haloalkyl, C₁-C₆ alkoxy, C₁-C₆ haloalkoxy, C₁-C₆ hydroxyalkyl, halogen, —NO₂, —CN, —OR₁₅, —SR₁₅, —C(O)R₁₅, —NHC(O)R₁₅, —C(O)OR₁₅, —OC(O)R₁₅, NR₁₆R₁₇, —C(O)NR₁₆R₁₇, —NHR₁₅C(O)NR₁₆R₁₇, —SO₂NR₁₆R₁₇, cycloalkyl, heterocyclyl, or heteroaryl, each being optionally substituted with one to four R₂₀,
R₂₀ is halogen, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxy, C₁-C₆ haloalkyl, C₁-C₆ alkoxy, C₁-C₆ haloalkoxy, —CO₂H, —CO₂(C₁-C₆ alkyl), —SO₂(C₁-C₆ alkyl), —CONH₂, —CONH(C₁-C₆ alkyl), —CON(C₁-C₆ alkyl)₂, —CON(H)OH, —NHCO(C₁-C₆ alkyl), or —NHCO₂(C₁-C₆ alkyl); and
m is an integer 1 or 2.

In yet another embodiment, the disclosure provides compounds of formula (IIa), wherein:
R₁₁ is C₁-C₆ alkyl, C₂-C₆ alkenyl, C₂-C₆ alkynyl, C₁-C₆ haloalkyl, C₁-C₆ alkoxy, C₁-C₆ hydroxyalkyl, C₁-C₆ haloalkoxy, halogen, —NO₂, —CN, —OR₁₅, —SR₁₅, —C(O)R₁₅, —NHC(O)R₁₅, —C(O)OR₁₅, —OC(O)R₁₅, —NR₁₆R₁₇, —C(O)NR₁₆R₁₇, —NHR₁₅C(O)NR₁₆R₁₇, —SO₂NR₁₆R₁₇, alkylaryl, cycloalkyl, heterocyclyl, or heteroaryl, each being optionally substituted with one to four R₂₀;
R₁₂ is H, C₁-C₆ alkyl, C₂-C₆ alkenyl, C₂-C₆ alkynyl, C₁-C₆ haloalkyl, C₁-C₆ alkoxy, C₁-C₆ hydroxyalkyl, C₁-C₆ haloalkoxy, halogen, —NO₂, —CN, —SR₁₅, —C(O)R₁₅, —NHC(O)R₁₅, —C(O)OR₁₅, —OC(O)R₁₅, —NR₁₆R₁₇, —C(O)NR₁₆R₁₇, —NHR₁₅C(O)NR₁₆R₁₇, —SO₂NR₁₆R₁₇, alkylaryl, cycloalkyl, heterocyclyl, or heteroaryl, each being optionally substituted with one to four R₂₀;
R₁₃ is C₁-C₆ alkyl, C₂-C₆ alkenyl, C₂-C₆ alkynyl, C₁-C₆ haloalkyl, C₁-C₆ alkoxy, C₁-C₆ hydroxyalkyl, C₁-C₆ haloalkoxy, halogen, —NO₂, —CN, —OR₁₅, —SR₁₅, —C(O)R₁₅, —NHC(O)R₁₅, —C(O)OR₁₅, —OC(O)R₁₅, —NR₁₆R₁₇, —C(O)NR₁₆R₁₇, —NHR₁₅C(O)NR₁₆R₁₇, —SO₂NR₁₆R₁₇, alkylaryl, cycloalkyl, heterocyclyl, or heteroaryl, each being optionally substituted with one to flour R₂₀;
R₁₄ is C₁-C₆ alkyl, C₂-C₆ alkenyl, C₂-C₆ alkynyl, aryl, C₁-C₆ alkylaryl, cycloalkyl, heterocyclyl, or heteroaryl, each being optionally substituted with one to four R₂₀, or
R₁₅, R₁₆, and R₁₇ are independently H, C₁-C₆ alkyl, C₂-C₆ alkenyl C₂-C₆ alkynyl, C₁-C₆ alkoxy, halogen, hydroxyl, aryl, alkylaryl, cycloalkyl, heterocyclyl, or heteroaryl, each being optionally substituted with one to four R₂₀;
R₁₈ and R₁₉ are each independently H, C₁-C₆ alkyl, C₂-C₆ alkenyl, C₂-C₆ alkynyl, C₁-C₆ haloalkyl, C₁-C₆ alkoxy, C₁-C₆ haloalkoxy, C₁-C₆ hydroxyalkyl, halogen, —NO₂, —CN, —OR₁₅, —SR₁₅, —C(O)R₁₅, —NHC(O)R₁₅, —C(O)OR₁₅, —OC(O)R₁₅, —NR₁₆R₁₇, —C(O)NR₁₆R₁₇, —NHR₁₅C(O)NR₁₆R₁₇, —SO₂NR₁₆R₁₇, alkylaryl, cycloalkyl, heterocyclyl, or heteroaryl, each being optionally substituted with one to four R₂₀,
R₂₀ is halogen, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxy, C₁-C₆ haloalkyl, C₁-C₆ alkoxy, C₁-C₆ haloalkoxy, —CO₂H, —CO₂(C₁-C₆ alkyl), —SO₂(C₁-C₆ alkyl), —CONH₂, —CONH(C₁-C₆ alkyl), —CON(C₁-C₆ alkyl)₂, —CON(H)OH, —NHCO(C₁-C₆ alkyl), or —NHCO₂(C₁-C₆ alkyl); and
m is an integer 1 or 2.

In another embodiment, the disclosure provides compounds of formula (IIa) wherein:
R₁₁ is C₁-C₆ alkyl, C₂-C₆ alkenyl, C₂-C₆ alkynyl, C₁-C₆ haloalkyl, C₁-C₆ alkoxy, C₁-C₆ hydroxyalkyl, C₁-C₆ haloalkoxy, halogen, —NO₂, —CN, —OR₁₅, —SR₁₅, —C(O)R₁₅, —NHC(O)R₁₅, —C(O)OR₁₅, —OC(O)R₁₅, —NR₁₆R₁₇, —C(O)NR₁₆R₁₇, —NHR₁₅C(O)NR₁₆R₁₇, —SO₂NR₁₆R₁₇, alkylaryl, cycloalkyl, heterocyclyl, or heteroaryl, each being optimally substituted with one to four R₂₀;
R₁₂ is H, C₁-C₆ alkyl, C₂-C₆ alkenyl, C₂-C₆ alkynyl, C₁-C₆ haloalkyl, C₁-C₆ alkoxy C₁-C₆ hydroxyalkyl, C₁-C₆ haloalkoxy, halogen, —NO₂, —CN, —OR₁₅, —SR₁₅, —C(O)R₁₅, —NHC(O)R₁₅, —C(O)OR₁₅, —OC(O)R₁₅, —NR₁₆R₁₇, —C(O)NR₁₆R₁₇, —NHR₁₅C(O)NR₁₆R₁₇, —SO₂NR₁₆R₁₇, alkylaryl, cycloalkyl, heterocyclyl, or heteroaryl, each being optionally substituted with one to four R₂₀;
R₁₃ is C₁-C₆ alkyl, C₂-C₆ alkenyl, C₂-C₆ alkynyl, C₁-C₆ haloalkyl, C₁-C₆ alkoxy, C₁-C₆ hydroxyalkyl, C₁-C₆ haloalkoxy, halogen, —NO₂, —CN, —OR₁₅, —SR₁₅, —C(O)R₁₅, —NHC(O)R₁₅, —C(O)OR₁₅, —OC(O)R₁₅, —NR₁₆R₁₇, —C(O)NR₁₆R₁₇, —NHR₁₅C(O)NR₁₆R₁₇, —SO₂NR₁₆R₁₇, alkylaryl, cycloalkyl, heterocyclyl, or heteroaryl, each being optionally substituted with one to four R₂₀;
R₁₄ is H;
R₁₅, R₁₆, and R₁₇ are independently H, C₁-C₆ alkyl, C₂-C₆ alkenyl, C₂-C₆ alkynyl, C₁-C₆ alkoxy, halogen, hydroxyl, aryl, alkylaryl, cycloalkyl, heterocyclyl, or heteroaryl, each being optionally substituted with one to four R₂₀;
R₁₈ and R₁₉ are each independently H, C₁-C₆ alkyl, C₂-C₆ alkenyl C₂-C₆ alkynyl, C₁-C₆ haloalkyl, C₁-C₆ alkoxy, C₁-C₆ haloalkoxy, C₁-C₆ hydroxyalkyl, halogen, —NO₂, —CN, —OR₁₅, —SR₁₅, —C(O)R₁₅, —NHC(O)R₁₅, —C(O)OR₁₅, —OC(O)R₁₅, —NR₁₆R₁₇, —C(O)NR₁₆R₁₇, —NHR₁₅C(O)NR₁₆R₁₇, —SO₂NR₁₆R₁₇, alkylaryl, cycloalkyl, heterocyclyl, or heteroaryl, each being optionally substituted with one to four R₂₀,
R₂₀ is halogen, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxy, C₁-C₆ haloalkyl, C₁-C₆ alkoxy. C₁-C₆ haloalkoxy, —CO₂H, —CO₂(C₁-C₆ alkyl), —SO₂(C₁-C₆ alkyl), —CONH₂, —CONH(C₁-C₆ alkyl), —CON(C₁-C₆ alkyl)₂, —CON(H)OH, —NHCO(C₁-C₆ alkyl), or —NHCO₂(C₁-C₆ alkyl); and
m is an integer 1 or 2.

In one embodiment, the disclosure as described above provides compounds wherein R₁₁ is C₁-C₆ alkyl, C₂-C₆ alkenyl, C₂-C₆ alkynyl, C₁-C₆ haloalkyl, C₁-C₆ alkoxy, C₁-C₆ hydroxyalkyl, C₁-C₆ haloalkoxy, halogen, —NO₂, —CN, —OR₁₅, —SR₁₅, —C(O)R₁₅, —NHC(O)R₁₅, —C(O)OR₁₅, NR₁₆R₁₇, —C(O)NR₁₆R₁₇, —SO₂NR₁₆R₁₇, or alkylaryl, each optionally substituted with one to four R₂₀. In another embodiment, R₁₁ is C₁-C₆ alkyl, C₁-C₆ haloalkyl, C₁-C₆ alkoxy, C₁-C₆ hydroxyalkyl, C₁-C₆ haloalkoxy, halogen, —NO₂, —CN, —OR₁₅, —C(O)R₁₅, —C(O)OR₁₅, —NR₁₆R₁₇, or —C(O)NR₁₆R₁₇. In yet another embodiment, R₁₁ is C₁-C₆ alkyl, C₁-C₆ haloalkyl, C₁-C₆ alkoxy, C₁-C₆ haloalkoxy, halogen, —NO₂, —CN, —OR₁₅, —C(O)R₁₅, —C(O)OR₁₅, —NR₁₆R₁₇, or —C(O)NR₁₆R₁₇. In some embodiments, R₁₁ is C₁-C₆ alkyl, C₁-C₆ haloalkyl, C₁-C₆ alkoxy, halogen, —OR₁₅, or —NR₁₆R₁₇.

In another embodiment, the disclosure as described above provides compounds wherein R₁₁ is C₁-C₆ alkyl or C₁-C₆ alkoxy. More specifically, R₁₁ is C₁-C₄ alkyl or C₁-C₄ alkoxy. Even more specifically, R₁₁ is methyl or methoxy. For example, R₁₁ is methyl.

In certain embodiments, the disclosure as described above provides compounds wherein R₁₂ is H or C₁-C₆ alkyl optionally substituted with R₂₀. In one embodiment, R₁₂ is H.

The disclosure as described above also provides compounds wherein R₁₃ is C₁-C₆ alkyl, C₂-C₆ alkenyl, C₂-C₆ alkynyl, C₁-C₆ haloalkyl, C₁-C₆ alkoxy, C₁-C₆ hydroxyalkyl, C₁-C₆ haloalkoxy, halogen, —NO₂, —CN, —OR₁₅, —SR₁₅, —C(O)R₁₅, —NHC(O)R₁₅, —C(O)OR₁₅, —NR₁₆R₁₇, —C(O)NR₁₆R₁₇, —SO₂NR₁₆R₁₇, or alkylaryl, each optimally substituted with one to four R₂₀. In another embodiment, R₁₃ is C₁-C₆ alkyl, C₁-C₆ haloalkyl, C₁-C₆ alkoxy, C₁-C₆ hydroxyalkyl, C₁-C₆ haloalkoxy, halogen, —NO₂, —CN, —OR₁₅, —C(O)R₁₅, —C(O)OR₁₅, —NR₁₆R₁₇, or —C(O)NR₁₆R₁₇. In yet another embodiment, R₁₃ is C₁-C₆ alkyl, C₁-C₆ haloalkyl, C₁-C₆ alkoxy, C₁-C₆ hydroxyalkyl, C₁-C₆ haloalkoxy, halogen, —NO₂, —CN, —OR₁₅, —C(O)R₁₅, —C(O)OR₁₅, —NR₁₆R₁₇, or —C(O)NR₁₆R₁₇. In some embodiments, R₁₃ is C₁-C₆ alkyl, C₁-C₆ haloalkyl, C₁-C₆ alkoxy, halogen, —OR₁₅, or —NR₁₆R₁₇.

In another embodiment, the disclosure as described above provides compounds wherein R₁₃ is C₁-C₆ alkyl. More specifically, R₁₃ is C₁-C₄ alkyl. Even more specifically, R₁₃ is methyl.

The disclosure as described above also provides compounds wherein R₁₃ and R₁₄ taken together with the atoms to which they are attached form a 5-, 6-, or 7-membered heterocyclyl group, each being optionally substituted with one to four R₂₀. In one embodiment, R₁₃ and R₁₄ taken together with the atoms to which they are attached form a 5-, or 6-membered heterocyclyl. In another embodiment, R₁₃ and R₁₄ taken together with the atoms to which they are attached form a 6-membered heterocyclyl (e.g., piperidinyl, piperazinyl, morpholinyl, etc.) In one embodiment, the heterocyclyl is piperidinyl.

In one embodiment, the disclosure as described above provides compounds wherein R₁₄ is H.

In another embodiment, the disclosure as described above provides compounds wherein R₁₄ is aryl, cycloalkyl, heterocyclyl, or heteroaryl, each being optionally substituted with one to four R₂₀. In yet another embodiment, R₁₄ is heterocyclyl or heteroaryl, each optionally substituted with R₂₀. In some embodiments, R₁₄ is heterocyclyl (e.g., piperidinyl, piperazinyl, morpholinyl, etc.) In one embodiment, R₁₄ is morpholinyl.

In another embodiment, the disclosure as described above provides compounds wherein R₁₄ is C₁-C₆ alkyl, C₂-C₆ alkenyl, or C₂-C₆ alkynyl, each being optionally substituted with one to four R₂₀. In yet another embodiment, R₁₄ is C₁-C₆ alkyl optionally substituted with one to four R₂₀. In yet another embodiment, R₁₄ is unsubstituted C₁-C₆ alkyl.

The disclosure as described above provides compounds wherein R₁₈ and R₁₉ are each independently H, C₁-C₆ alkyl, C₁-C₆ haloalkyl, C₁-C₆ alkoxy, C₁-C₆ haloalkoxy, C₁-C₆ hydroxyalkyl, halogen, —NO₂, —CN, —OR₁₅, —SR₁₅, —C(O)R₁₅, —C(O)OR₁₅, —NR₆R₁₇, —C(O)NR₁₆R₁₇, or —SO₂NR₁₆R₁₇, each being optionally substituted with one to four R₂₀. In one embodiment, R₁₈ and R₁₉ we each independently H, C₁-C₆ alkyl, C₁-C₆ haloalkyl, C₁-C₆ alkoxy, C₁-C₆ haloalkoxy, halogen, —NO₂, —CN, —OR₁₅, or NR₁₆R₁₇. In another embodiment, R₁₈ and R₁₉ are each independently H, alkyl, C₁-C₆ haloalkyl, C₁-C₆ alkoxy, C₁-C₆ haloalkoxy, halogen, or —OH.

The disclosure as described above also provides compounds R₁₃ and R₁₉ are each independently H, C₁-C₆ alkoxy, C₁-C₆ haloalkoxy, halogen, or —OH. In one embodiment, one of R₁₈ and R₁₉ is H, and the other is C₁-C₆ alkoxy, C₁-C₆ haloalkoxy, halogen, or —OH. In another embodiment, one of R₁₁ and R₁₉ is H, and the other is C₁-C₆ alkoxy or halogen. In yet another embodiment, one of R₁₈ and R₁₉ is H, and the other is halogen. In some embodiments, both R₁₈ and R₁₉ are H. In other embodiments, both R₁₈ and R₁₉ are halogen.

In certain specific embodiments, the disclosure as described above provides compounds wherein R₁₄ and R₁₈ taken together with the atoms to which they are attached form a 5-, 6-, or 7-membered cycloalkyl, heterocyclyl, aryl, or heteroaryl group, each being optionally substituted with one to four R₂₀. In one embodiment, R₁₄ and R₁₈ taken together with the atoms to which they are attached form a 6-membered heterocyclyl group, optionally substituted with R₂₀.

In certain embodiments, the disclosure provides compounds of formula (IIa) wherein R₁₄ is H. In other embodiments, R₁₁ is C₁-C₆ alkyl; R₁₃ is H, R₁₃ is C₁-C₆ alkyl; R₁₄ is H; and R₁₈ and R₁₉ are each independently H, C₁-C₆ alkyl, halogen, or —OH.

In other embodiments, the disclosure provides compounds of formula (IIa) wherein R₁₄ is heterocyclyl or heteroaryl, each optionally substituted with R₂₀. In some other embodiments, R₁₁ is C₁-C₆ alkyl; R₁₂ is H, R₁₃ is C₁-C₆ alkyl; R₁₄ is heterocyclyl or heteroaryl, each optionally substituted with R₂₀; and R₁₈ and R₁₉ are each independently H, C₁-C₆ alkyl, halogen, or —OH.

In other embodiments, the disclosure provides compounds of formula (IIa) wherein R₁₁ is C₁-C₆ alkyl; R₁₂ is H; R₁₃ and R₁₄ taken together with the atoms to which they are attached form a 5-, or 6-membered heterocyclyl, and R₁₈ and R₁₉ are each independently H, C₁-C₆ alkyl, halogen, or —OH.

In one embodiment, the disclosure provides compounds of formula (II), which is:

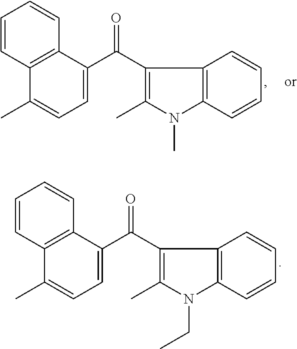 [SMILES: Cc1ccc(C(=O)c2c(C)n(C)c3ccccc23)c2ccccc12 ; CCn1c(C)c(C(=O)c2ccc(C)c3ccccc23)c2ccccc21]

In one embodiment, the disclosure provides compounds of formula (II), which is:

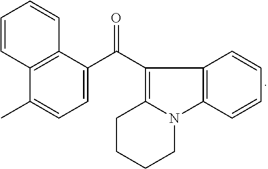 [SMILES: Cc1ccc(C(=O)c2c3n(c4ccccc24)CCCC3)c2ccccc12]

In one embodiment, the disclosure provides compounds of formula (II), which is:

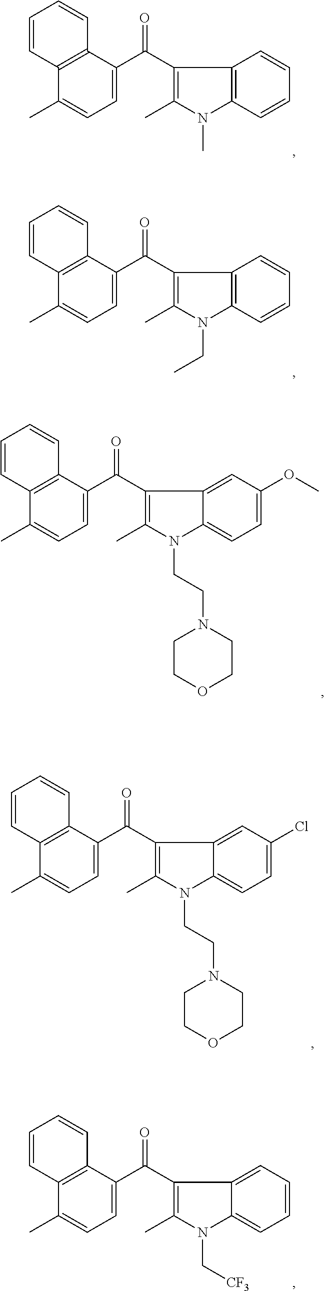 [SMILES: Cc1ccc(C(=O)c2c(C)n(C)c3ccccc23)c2ccccc12 ; CCn1c(C)c(C(=O)c2ccc(C)c3ccccc23)c2ccccc21 ; COc1ccc2c(c1)c(C(=O)c1ccc(C)c3ccccc13)c(C)n2CCN1CCOCC1 ; Cc1ccc(C(=O)c2c(C)n(CCN3CCOCC3)c3ccc(Cl)cc23)c2ccccc12 ; Cc1ccc(C(=O)c2c(C)n(CC(F)(F)F)c3ccccc23)c2ccccc12]

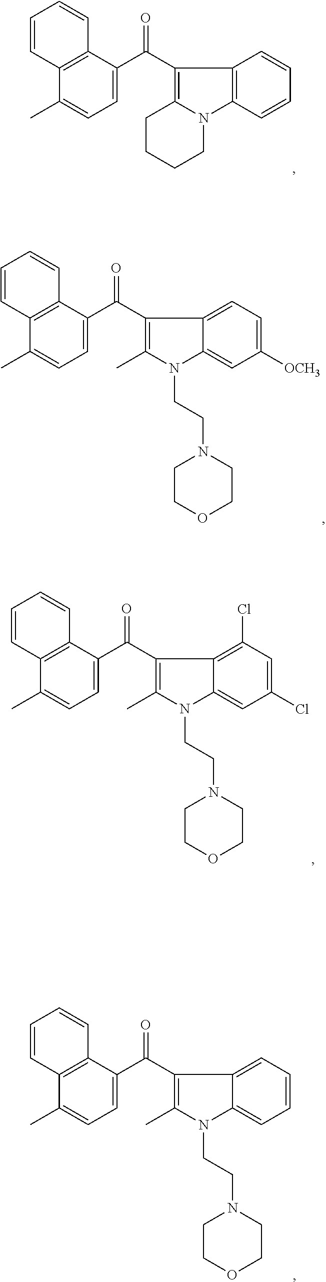 [SMILES: Cc1ccc(C(=O)c2c3n(c4ccccc24)CCCC3)c2ccccc12 ; COc1ccc2c(C(=O)c3ccc(C)c4ccccc34)c(C)n(CCN3CCOCC3)c2c1 ; Cc1ccc(C(=O)c2c(C)n(CCN3CCOCC3)c3cc(Cl)cc(Cl)c23)c2ccccc12 ; Cc1ccc(C(=O)c2c(C)n(CCN3CCOCC3)c3ccccc23)c2ccccc12]

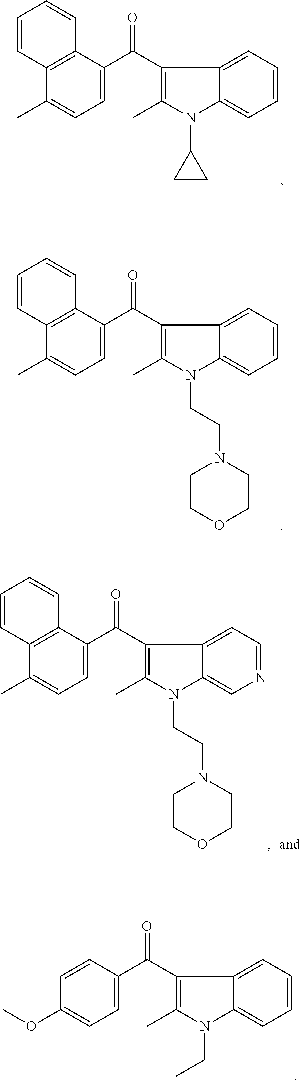 [SMILES: Cc1ccc(C(=O)c2c(C)n(C3CC3)c3ccccc23)c2ccccc12 ; Cc1ccc(C(=O)c2c(C)n(CCN3CCOCC3)c3ccccc23)c2ccccc12 ; Cc1ccc(C(=O)c2c(C)n(CCN3CCOCC3)c3cnccc23)c2ccccc12 ; CCn1c(C)c(C(=O)c2ccc(OC)cc2)c2ccccc21]

### Pharmaceutical Formulations and Modes of Administration

In various aspects, the disclosure provides a pharmaceutical composition comprising a therapeutically effective amount of a compound of the disclosure, and one or more pharmaceutically acceptable diluents, preservatives, solubilizers, emulsifiers, adjuvants, excipients, or carriers.

In certain aspects, the disclosure provides for a pharmaceutical composition comprising the compounds of the disclosure together with one or more pharmaceutically acceptable excipients or vehicles, and optionally other therapeutic and/or prophylactic ingredients. Such excipients include liquids such as water, saline glycerol, polyethyleneglycol, hyaluronic acid, ethanol, and the like.

The term “pharmaceutically acceptable vehicle” refers to a diluent, adjuvant, excipient or carrier with which a compound of the disclosure is administered. The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. For example, an “effective amount” for therapeutic uses is the amount of the composition comprising one or more polyene ma fide compounds disclosed herein required to treat diseases caused by fungal infections to provide a clinically significant decrease in infections. An appropriate “effective” amount in any individual case can be determined by one of ordinary skill in the art using routine experimentation.

“Pharmaceutically acceptable carriers” for therapeutic use are well known in the pharmaceutical art, and are described, for example, in Remington's Pharmaceutical Sciences, 18th Edition (Easton, Pa.: Mack Publishing Company, 1990). For example, sterile saline and phosphate-buffered saline at physiological pH can be used. Preservatives, stabilizers, dyes and even flavoring agents can be provided in the pharmaceutical composition. For example, sodium benzoate, sortie acid and esters of p-hydroxybenzoic acid can be added as preservatives. Id. at 1449. In addition, antioxidants and suspending agents can be used. Id.

Suitable excipients for non-liquid formulations are also known to those of skill in the art. A thorough discussion of pharmaceutically acceptable excipients and salts is available in Remington's Pharmaceutical Sciences, 18th Edition (Easton, Pa.: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline and the like.

Depending on the intended mode of administration, the pharmaceutical compositions can be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, can include other pharmaceutical agents, adjuvants, diluents, buffers, and the like.

The disclosure includes a pharmaceutical composition comprising a compound of the disclosure including isomers, racemic or non-racemic mixtures of isomers, or pharmaceutically acceptable salts or solvates thereof together with one or more pharmaceutically acceptable carriers, and optionally other therapeutic and/or prophylactic ingredients.

In general, the compounds of the disclosure will be administered in a therapeutically effective amount by any of the accepted modes of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compounds of the disclosure for a given disease.

Thus, the compounds of the disclosure can be administered as pharmaceutical formulations including those suitable for oral (including buccal and sub-lingual), rectal, nasal, topical, pulmonary, vaginal or parenteral (including intramuscular, intra-arterial, intrathecal, subcutaneous and intravenous) administration or in a form suitable for administration by inhalation or insufflation. The preferred manner of administration is intravenous or oral using a convenient daily dosage regimen which can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art for example, see Remington's Pharmaceutical Sciences, referenced above.

In yet another embodiment is the use of permeation enhancer excipients including polymers such as: polycations (chitosan and its quaternary ammonium derivatives, poly-L-arginine, aminated gelatin); poly-anions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

For oral administration, the composition will generally take the form of a tablet, capsule, a softgel capsule or can be an aqueous or nonaqueous solution, suspension or syrup. Tablets and capsules are preferred oral administration forms. Tablets and capsules for oral use can include one or more commonly used carriers such as lactose and corn starch. Lubricating agents, such as magnesium stearate, are also typically added. Typically, the compounds of the disclosure can be combined with an oral, nontoxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Thus, for example, capsules can be prepared by conventional procedures so that the dosage unit is 100 mg of the compounds of the disclosure, 100 mg of cellulose and 10 mg of magnesium stearate. A large number of unit capsules can also prepared by filling standard two-piece hard gelatin capsules each with 100 mg of powdered active ingredient, 150 mg of lactose, 50 mg of cellulose, and 10 mg magnesium stearate. Or, tablets can be prepared by conventional procedures so that the dosage unit is 100 mg of the compounds of the disclosure, 150 mg of lactose, 50 mg of cellulose and 10 mg of magnesium stearate. A large number of tablets can also be prepared by conventional procedures such that the dosage unit was 100 mg of the compounds of the disclosure, and other ingredients can be 0.2 mg of colloidal silicon dioxide, 5 mg of magnesium stearate, 250 mg of microcrystalline cellulose, 10 mg of starch and 100 mg of lactose. Appropriate coatings can be applied to increase palatability or delay absorption.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms sinkable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as an continuous infusion system. A formulation provided by the disclosure can be administered liming a syringe, injector, pump, or any other device recognized in the art for parenteral administration.

Preferably, sterile injectable suspensions are formulated according to techniques known in the art using suitable carriers, dispersing or wetting agents and suspending agents. The sterile injectable formulation can also be a steak injectable solution or a suspension in a nontoxic parenterally acceptable diluent or solvent. Among the acceptable vehicles and solvents that can be employed are water, Ringer's solution and isotonic sodium chloride solution. In addition, suede, fixed oils, fatty esters or polyols are conventionally employed as solvents or suspending media. In addition, parenteral administration can involve the use of a slow release or sustained release system such that a constant level of dosage is maintained.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the composition, by irradiating the compositions, or by heating the compositions. They can also be manufactured using sterile water, or some other sterile injectable medium, immediately before use.

The formulations can optionally contain an isotonicity agent. The formulations preferably contain an isotonicity agent and glycerin is the most preferred isotonicity agent. The concentration of glycerin, when it is used, is in the range known in the art, such as for example, about 1 mg/mL to about 20 mg/mL.

The pH of the parenteral formulations can be controlled by a buffering agent such as phosphate, acetate, TRIS or L-arginine. The concentration of the buffering agent is preferably adequate to provide buffering of the pH during storage to maintain the pH at a target pH±0.2 pH unit. The preferred pH is between about 7 and about 8 when measured at room temperature.

Other additives, such as a pharmaceutically acceptable solubilizers like Tween 20® (polyoxyethylene (20) sorbitan monolaurate), Tween 40® (polyoxyethylene (20) sorbitan monopalmitate), Tween 80® (polyoxyethylene (20) sorbitan monooleate), Pluronic F68® (polyoxyethylene polyoxypropylene block copolymers), and PEG (polyethylene glycol) can optionally be added to the formulation, and can be useful if the formulations will contact plastic materials. In addition, the parenteral formulations can contain various antibacterial and antifungal agents, for example, parabens, chlorobutanol, phenol, sorbic acid, thimerosal, and the like.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Alternatively, the pharmaceutical compositions of the disclosure can be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax and polyethylene glycols.

The pharmaceutical compositions of the disclosure can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, propellants such as fluorocarbons or nitrogen, and/or other conventional solubilizing or dispersing agents.

Preferred formulations for topical drug delivery are ointments and creams. Ointments are semisolid preparations which are typically based on petrolatum or other petroleum derivatives. Creams containing the selected active agent, are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base ID be used, as will be appreciated by those skilled in the art, is one that will provide for optimum drug delivery. As with other carriers or vehicles, an ointment base should be inert, stable, nonirritating and nonsensitizing.

Formulations for buccal administration include tablets, lozenges, gels and the like. Alternatively, buccal administration can be effected using a transmuscosal delivery system as known to those skilled in the art. The compounds of the disclosure can also be delivered through the skin or muscosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device can contain a single reservoir, or it can contain multiple reservoirs. In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, can be either a polymeric matrix as described above, or it can be a liquid or gel reservoir, or can take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

The compounds of the disclosure can be formulated for aerosol administration, particularly to the respiratory tract and including intranasal administration. The compound will generally have a small particle size for example of the order of 5 microns or less. Such a particle size can be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol can conveniently also contain a surfactant such as lecithin. The dose of ding can be controlled by a metered valve. Alternatively the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form for example in capsules or cartridges of e.g., gelatin or blister packs from which the powder can be administered by means of an inhaler.

A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. For purposes of the disclosure, generally a therapeutic amount will be in the range of about 0.01 mg/kg to about 250 mg/kg body weight, more preferably about 0.1 mg/kg to about 10 mg/kg, in at least one dose. In larger mammals the indicated daily dosage can be from about 1 mg to 300 mg, one or more times per day, more preferably in the range of about 10 mg to 200 mg. The subject can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

The pharmaceutical preparations are preferably in unit dosage forms. In such farm, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself or it can be the appropriate number of any of these in packaged form.

### Methods of Treating of Glioblastoma

WIN 55212-2 (WIN-2; ST-1) was reported to bind with nanomolar affinity to a protein expressed by NG108-15 cells. CP55,940 (CP), a high-affinity agonist at CB₁ and CB₂ receptors, did not compete for WIN-2 binding in these cells. WIN-2 was reported to increase [³⁵S]-GTPγS binding in homogenates prepared from CB₁−/− mice cerebellum, a response insensitive to the CB₂ antagonist SR144528. WIN-2 was also reported to inhibit excitatory transmission in hippocampal slices prepared from CB₁−/− mice, and subsequently showed that this response is blocked by the TRPV1 antagonist capsazepine. Together, these findings suggest that a receptor activated by the aminoalkylindole compound WIN-2 exists and that it is a G protein-coupled receptor (GPCR).

G protein-coupled receptors (GPCRs) constitute a family of proteins sharing a common structural organization characterized by an extracellular N-terminal end, seven hydrophobic alpha helices putatively constituting transmembrane domains, and an intracellular C-terminal domain. GPCRs bind a wide variety of ligands that bigger intracellular signals through the activation of transducing G proteins. More than 300 GPCRs have been cloned, and it is generally assumed that well over 1,000 of such receptors exist. Roughly 50-60% of all clinically relevant drugs act by modulating the functions of various GPCRs.

Activation of CB₁ and CB₂ receptors kill astrocytomas. In the mid-1990s, CB₁ receptors were reported to be present in various human glioma cell lines, as well as explants of human tumors with various degrees of malignancy. Accordingly, agonists at CB₁ receptors active the ERR kinase pathway and transcription factor krox-24 in human glioma cell lines in culture, responses antagonized by the CB₁ receptor antagonist rimonabant applied a nanomolar concentrations. Shortly after these publications, cannabinoids were hypothesized to serve as powerful anti-tumoral agents in the treatment of astrocytomas.

As the prototypical AI compound, WIN-2, was originally synthesized as an anti-inflammatory and analgesic agent. Its serendipitous pharmacological targeting of CB₁/CB₂ has saved as a highly efficacious tool to study cannabinoid signaling, and more interestingly to our study, has revealed non-CB₁CB₂-mediated effects. The disclosure shows that mouse and human astrocytomas cell line express GPR124, a AI receptor and that agonists at GPR124 receptor selectively kill tumor cells without harming healthy cells.

The disclosure provides methods of treating or inhibiting glioblastoma in a subject, the method comprising administering to the subject an effective amount of a compound as discussed above.

In another aspect, the disclosure provides for methods of activating the GPR124 receptor comprising administering a compound of formula as defined above.

In various embodiments, the compounds of the disclosure bind to GPR124. In further embodiments, the compounds of the disclosure bind to no more than one of the CB₁ or CB₂ cannabinoid receptors. In some embodiments, the compounds of the disclosure do not bind to the CB₁ or CB₂ cannabinoid receptors. In certain embodiments, the disclosure provides methods where astrocytomas are killed.

GPR124 was initially identified in endothelial cells derived from blood vessels growing in colorectal tumors. No compound acting through this receptor has been reported yet and its signal transduction mechanism is only starting to be delineated. Genetic approaches aimed at deleting or ova-expressing GPR124 in selective cell populations show that this receptor plays a crucial role in the development of vasculature and the migration of endothelial cells. While the expression pattern of GPR124 in healthy human brain and in human GBMs still needs to be determined, the mouse brain atlas of the Allen Institute indicates that GPR124 is expressed at low level in healthy mouse brain.

In one embodiment, the compounds of the disclosure as defined above selectively bind to GPR124. As defined herein, the term “selectively binds” means binding to a predetermined target where the dissociation constant is at least two orders of magnitude lower than the dissociation constant of the non-binding targets. In another embodiment, the compounds of the disclosure as defined above activate no MOW than one of the CB₁ or CB₂ cannabinoid receptors. As defined herein, the term “activate” means having an increased activity, i.e., agonist. Compounds with decreased activity ‘inhibit”, i.e., antagonist. In yet another embodiment, the compounds of the disclosure as defined above do not functionally activate or inhibit the CB₁ or CB₂ cannabinoid receptors.

The disclosure also provides methods of treating glioblastomas in a subject comprising activating the GPR124 receptor in the brain of the subject, comprising administering one or more of compounds of the disclosure as described above. In one embodiment, astrocytomas are killed. In additional embodiment, the CB₁ or CB₂ cannabinoid receptors are not activated or inhibited by the treatment.

The disclosure further provides methods of enhancing or reducing GPR124 activity in a subject comprising administering an agonist of GPR124 in the brain of the subject. In further embodiment, the CB₁ or CB₂ cannabinoid receptors are not activated by the agonist. In one embodiment, the agonist is the compound of the disclosure as described above.

### Screening Methods

In one aspect, the disclosure provides methods of screening for therapeutic agents useful in the treatment of glioblastomas or melanoma in a subject, comprising the steps of: contacting a test compound with a GPR124 polypeptide or a fragment thereof;
measuring a signal correlated with binding of the test compound to the GPR124 polypeptide; contacting the test compound with the CB₁ cannabinoid receptor,
measuring a signal correlated with binding of the test compound to the CB₁ cannabinoid receptor,
contacting the test compound with the CB₂ cannabinoid receptor,
measuring a signal correlated with binding of the test compound to the CB₂ cannabinoid receptor; and
determining whether the test compound binds to the GPR124 polypeptide, CB₁ cannabinoid receptor, and CB₂ cannabinoid receptor, and
selecting a positive test compound that binds to the GPR124 polypeptide but not to one of the CB₁ or CB₂ cannabinoid receptors.

In one embodiment, the test compound is an agonist of GPR124. In further aspects, the test compound is an antagonist of GPR124. In another embodiment the test compound is the compound of disclosure as described above.

In one embodiment, the disclosure provides methods of screening for therapeutic agents further comprising:
contacting a second test agent with a GPR124 polypeptide or a fragment thereof, wherein the GPR124 is bound to the positive test compound;
measuring a signal correlated with binding of the positive test compound to the GPR124 polypeptide;
selecting a second test compound that modulates the activity of the positive test compound at the GPR124 polypeptide.

In one embodiment, the step of contacting is in or at the surface of a cell. In other embodiment, the step of contacting is in a cell-free system.

In certain embodiments, the polypeptide is coupled to a detectable label. In other embodiments, the test compound is coupled to a detectable label. In another embodiment, the test compound displaces a ligand which is first bound to the polypeptide.

In one embodiment, the test compound is an agonist of GPR124. In further aspects, the test compound is an antagonist of GPR124. In another embodiment; the test compound is the compound of the disclosure as described above.

In certain embodiments, the polypeptide is protein.

### Kits

In other aspects, the disclosure provides for kits that can be used to perform the methods described herein. In various aspects, the kits comprise the compounds of the disclosure in one or more containers. In some aspects, the kits contain all of the components necessary and/or sufficient to administer the compounds of time disclosure to a subject, including instructions for administering the compounds. In some aspects, the kits contain all of the components necessary and/or sufficient to perform a the assays of the screening methods of the disclosure, including all controls, directions for performing assays, and any necessary software for analysis and presentation of results. In certain aspects, the disclosure provides for a compartment kit in which reagents are contained in separate containers. Such containers allow one to efficiently transfer reagents from one compartment to another compartment such that the samples and reagents are not cross-contaminated, and the agents or solutions of each container can be added in a quantitative fashion from one compartment to another. Such containers will include a container which will accept the test sample, a container which contains the soluble receptor used in the methods, containers which contain wash reagents (such as phosphate buffered saline, Tris-buffs, and the like), and containers which contain the reagents used to detect signals corresponding to binding of the CB₁ and CB₂ receptors and the GPR124 receptor. One skilled in the art will readily recognize that the presently disclosed compounds can be readily incorporated into one of the established kit formats which are well known in the art.

In one embodiment, the disclosure provides for a kit comprising a compound of the disclosure as described above. In another embodiment, the disclosure provides for a kit comprising a compound of the disclosure as described above.

### Other Therapeutic Methods

The disclosure also provides embodiments related to the interaction between AIs compounds and other ligands and the AI receptors. According to the disclosure, methods for the identification of compounds that modulate the binding of AIs and other ligands to AI receptors are provided. These methods are used to identify compounds that modulate AI compounds and other ligand activation of AI receptors, identify compounds that are agonists, antagonists, allosteric modulators, or inverse agonists of AI receptors, and identify compounds that selectively modulate AI receptors, rather than other receptors, such as CB₁ or CB₂. Assays of the disclosure can also be used to identify compounds having activity at any combination of CB₁, CB₂ and AI receptors.

Modulation of the AI binding site activity by endogenous, natural or synthetic agonists, antagonists or inverse agonists may be useful for the treatment (therapeutic or prophylactic) of a number of diseases where cannabinoid-hie ligands play a key role or have a beneficial effect, in particular but not limited to tissues where AI binding site is expressed and where AI are implicated to have a significant disease modifying effect, such as the prefrontal cortex, substantia nigra and nucleus basalis of Meynert in CNS and cognition disorders, ego schizophrenia, Alzheimer's disease and dementia, or the caudate and putamen in Parkinson's disease, depression, multiple sclerosis, and other pathologies associated with neuroinflammation amyotrophic lateral sclerosis (ALS), Huntington's disease, Fronto temporal dementia, parkinsonism linked to chromosome 17 and prion diseases such as Kuru, Creutzfeld-Jacob disease, scrapie and bovine spongiform encephalitis, and the like). Thus, the disclosure provides method of treatment of acognition disorders, schizophrenia, Alzheimer's disease and dementia, Parkinson's disease, depression, multiple sclerosis, amyotrophic lateral sclerosis (ALS), Huntington's disease, Fronto temporal dementia, parkinsonism linked to chromosome 17, and prion diseases (such as Kuru, Creutzfeld-Jacob disease, scrapie and bovine spongiform encephalitis) comprising administering to the subject an effective amount of a compound of the disclosure as described above.

The disclosure also provides method of treatment of cancer comprising administering to the subject an effective amount of a compound of the disclosure as described above. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, melanoma, breast cancer, medulloblastomas, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer. In one embodiment, cancer is melanoma, breast cancer, medulloblastomas, astrocytoma, and colon cancer.

Modulation of closely related GPCR and of other therapeutic targets (including proteins involved in the pathogenesis of a specific disease) by compounds of the disclosure as described above may be useful for the treatment (therapeutic or prophylactic) of a number of diseases where these related GPCRs and other therapeutic targets are implicated to have a significant disease modifying effect.

### DEFINITIONS

Any terms not directly defined herein shall be understood to have the meanings commonly associated with them as understood within the art of the disclosure. Certain terms are discussed herein to provide additional guidance to the practitioner in describing the compositions, devices, methods, and the like, of embodiments of the disclosure, and how to make or use them. It will be appreciated that the same thing can be said in more than one way. Consequently, alternative language and synonyms can be used for any one or more of the terms discussed herein. No significance is to be placed upon whether or not a term is elaborated or discussed herein. Some synonyms or substitutable methods, materials and the like are provided. Recital of one or a few synonym or equivalents does not exclude use of other synonyms or equivalents, unless it is explicitly stated. Use of examples, including examples of terms, is for illustrative purposes only and does not limit the scope and meaning of the embodiments of the disclosure herein.

As used in the specification, the singular forms “a,” “an” and “the” include plural referents unless the context clearly dictates otherwise.

As used herein, the term “patient” or “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to birds, fish and the like. The term does not denote a particular age or gender.

Chemical moieties referred to as univalent chemical moieties (e.g., alkyl, aryl, and the like) also encompass structurally permissible multivalent moieties, as understood by those skilled in the art. For example, while an “alkyl” moiety generally refers to a monovalent radical (e.g., CH₃CH₂—), in appropriate circumstances an “alkyl” moiety can also refer to a divalent radical (e.g., —CH₂CH₂—, which is equivalent to an “alkylene” group). Similarly, under circumstances where a divalent moiety is required, those skilled in the art will understand that the term “aryl” refers to the corresponding divalent arylene group.

Terms used herein may be preceded and/or followed by a single dash, “-”, or a double dash, “=”, to indicate the bond order of the bond between the named substituent and is parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, C₁-C₆alkoxycarbonyloxy and —OC(O)C₁-C₆alkyl indicate the same functionality; similarly arylalkyl and -alkylaryl indicate the same functionality.

All atoms are understood to have their normal number of valences for bond formation (e.g., 4 for carbon, 3 for N, 2 for O, and 2, 4, or 6 for S, depending on the atom's oxidation state). On occasion a moiety can be defined, for example, as (A)₉B, wherein a is 0 or 1. In such instances, when a is 0 the moiety is B and when a is 1 the moiety is AB.

Where a substituent can vary in the number of atoms or groups of the same kind (e.g., alkyl groups can be C₁, C₂, C₃, and the like), the number of repeated atoms or groups can be represented by a range (e.g., C₁-C₆ alkyl) which includes each and every number in the range and any and all sub ranges. FOE example, C₁-C₃ alkyl includes C₁, C₂, C₃, C₁₋₂, C₁₋₃, and C₂₋₃ alkyl.

“Alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, —CH₂CH(CH₂CH₃)CH₂—.

The terra “alkenyl” as used herein, means a straight branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2 propenyl 3-butenyl, 4-pentenyl, 5-hexenyl, 2-heptenyl, 2-methyl-1-heptenyl, 3-decenyl, and 3,1-dimethylocta-2,6-dienyl.

The term “alkynyl” as used herein, means a straight or branched chain hydrocarbon group containing from 2 to 10 carbon atoms and containing at least one carbon-carbon triple bond. Representative examples of alkenyl include, but are not limited, to acetylenyl, 1-propynyl, 2-propynyl, 3-butynyl, 2-pentynyl, and 1-butynyl.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocycle heterocyclyl portions of the bicyclic aryl are optionally substituted with one or two oxo and/or this groups. Representative examples of the bicyclic aryls include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl 2H-chromes-2-on-7-yl, 2H-chromen-2-on-8-yl, isoindoline-1,3-dion-4-yl, isoindoline-1,3-dion-5-yl, index-1-on-4-yl, inden-1-on-5-yl, inden-1-on-6-yl, inden-1-an-7-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-an-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fined cycloalkyl, cycloalkenyl, and heterocyclyl groups are optionally substituted with one or two groups which are independently oxo or thia.

An “aralkyl” or “arylalkyl” group comprises an aryl group covalently attached to an alkyl group, either of which independently is optionally substituted. Preferably, the aralkyl group is aryl(C₁-C₆)alkyl, including, without limitation, benzyl, phenethyl, and naphthylmethyl.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted with one or two groups which are independently oxo or thia. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia.

The term “heterocyclyl” as used herein, means a monocyclic heterocycle or a bicyclic heterocycle. The monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, imidazolinyl, imidazolidinyl, isothiazolinyl, isothiazolidinyl, isoxazolinyl, isoxazolidinyl, morpholinyl oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to either a phenyl, a monocyclic cycloalkyl, a monocyclic cycloalkenyl, a monocyclic heterocycle, or a monocyclic heteroaryl. The bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. Heterocyclyl groups are optionally substituted with one or two groups which are independently oxo or thia. In certain embodiments, the bicyclic heterocyclyl is a 5 or 6 membered monocyclic heterocyclyl ring fused to phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia.

“Halogen” refers to a chloro, bromo, fluoro or iodo atom radical. The terra “halogen” also contemplates terms “halo” or “halide”.

The terms “haloalkyl”, “haloalkenyl” and “haloalkoxy” refer to an alkyl, alkenyl or alkoxy group, as the case may be, which is substituted with one or more halogen atoms.

“Heteroatom” refers to a non-carbon atom, where boron, nitrogen, oxygen, sulfur and phosphorus are preferred heteroatoms, with nitrogen, oxygen and sulfur being particularly preferred heteroatoms in the compounds of the disclosure.

The terra “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a phenyl ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia.

“Hydroxyalkyl” refers to a branched or unbranched alkyl group bearing a hydroxy (—OH) group. Examples include hydroxymethyl (—CH₂OH, a C₁hydroxyalkyl) and 1-hydroxyethyl (—CHOHCH₃, a C₂hydroxyalkyl).

The term “nitro” as used herein, means a —NO₂ group.

The term “oxo” as used herein means a ═O group.

The term “saturated” as used herein means the referenced chemical structure does not contain any multiple carbon-carbon bonds. For example, a saturated cycloalkyl group as defined herein includes cyclohexyl, cyclopropyl, and the like.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

The phrase “one or more substituents”, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

The term “substituted”, as used herein, means that a hydrogen radical of the designated moiety is replaced with the radical of a specified substituent, provided that the substitution results in a stable or chemically feasible compound. Unless otherwise indicated, an optionally substituted group may have a substituent at each substitutable position of the group, and the substituents may be either the same or different.

Examples of suitable substituents on the unsaturated carbon atom of an aryl or heteroaryl group include -halo, —NO₂, —CN, —R′, —C(R′)═C(R′)₂, —C≡C—R′, —OR′, —SR′″, —S(O)R′″, —SO₂R′″, —SO₂N(R″)₂, —N(R″)₂, —NR″C(O)R′, —NR″C(O)N(R″)₂, —NR″CO₂R′″, —O—CO₂R″, —OC(O)N(R″)₂, —C—C(O)R′, —CO₂R′, —C(O)—C(O)R′, —C(O)R′, —C(O)N(R″)₂, —C(═NR″)—N(R″)₂, —C(═NR″)—OR′, —N(R″)—N(R″)₂, —N(R″)C(—NR″)—N(R″)₂, —NR″SO₂R′″, —NR″SO₂N(R″), —P(O)(R′)₂, —P(O)(R′)₂, —O—P(O)—OR′, and —P(O)(NR″)—N(R″)₂, wherein R′″ is an aliphatic or aryl group, and R′ and R″ are independently hydrogen, alkyl, alkenyl, alkynyl, haloalkyl, haloalkenyl, haloaklynyl, cycloalkyl, cycloalkenyl, aryl, heteroaryl, or heterocyclyl, or two adjacent substituents, taken together with their intervening atoms, farm a 5- to 6-membered saturated or partially unsaturated ring having 0-3 ring atoms selected from the group consisting of N, O, and S.

Examples of suitable substituents on the saturated carbon of an aliphatic group or of a non-aromatic ring include, without limitation, those listed above and the following: ═O, ═S, ═C(R′)₂, ═N—N(R″)₂, ═N—OR′, ═N—NHC(O)R′, ═N—NHCO₂R′″, ═N—NHSO₂R′″, or ═N—R′ where each R′, R″, and R′″ is as defined above. For the purposes of clarity, the term “substituted aliphatic” refers to an aliphatic group having at least one non-aliphatic substituent.

Suitable substituents on a substitutable nitrogen atom of a heteroaryl or heterocyclic ring include —R′, —N(R′)₂, —C(O)R′, —CO₃R′, —C(O)—C(O)R′, —C(O)CH₂C(O)R′, —SO₂R′, —SO₂N(R′)₂, —C(═S)N(R′)₂, —C(═NH)—N(R′)₂, and —NR′SO₂R′; wherein each R′ is as defined above.

Compounds of the disclosure can exist as stereoisomers, wherein asymmetric or chiral centers are present. Stereoisomers are designated (R) or (S) depending on the configuration of substituents around the chiral carbon atom. The terms (R) and (S) used herein are configurations as defined in IUPAC 1974 Recommendations for Section E, Fundamental Stereochemistry, Pure Appl. Chem., (1976), 45: 13-30, hereby incorporated by reference. The disclosure contemplates various stereoisomers and mixtures thereof, which are specifically included within the scope of the disclosure. Stereoisomers include enantiomers, diastereomers, and mixtures of enantiomers or diastereomers. Individual stereoisomers of compounds of the disclosure can be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Also, moieties disclosed herein which exist in multiple tautomeric forms include all such forms encompassed by a given tautomeric structure.

“Pharmaceutically acceptable” means approved or approvable by a regulatory agency of the Federal or state government or listed in the U.S. Pharmacopoeia or other generally recognized pharmacopoeia for use in animals, and more particularly in humans. It can be material which is not biologically or otherwise undesirable, i.e., the material can be administered to an individual without causing any undesirable biological effects or interacting in a deleterious manner with any of the components of the composition in which it is contained.

The term “pharmaceutically acceptable salt” of a compound means a salt that is pharmaceutically acceptable and that possesses the desired pharmacological activity of the parent compound. Such salts include, for example, acid addition salts and base addition salts.

“Acid addition salts” according to the disclosure, are formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, butyl sulfuric acid, gluconic acid, glutamic acid, hydroxynaphthoic acid, salicylic acid, stearic acid, muconic acid, and the like.

“Base addition salts” according to the disclosure are formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. It should be understood that a reference to a pharmaceutically acceptable salt includes the solvent addition forms or crystal forms thereof particularly solvates or polymorphs. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various actors such as the recrystallization solvent, rate of crystallization, and storage temperature can cause a single crystal form to dominate.

The term “agonist” refers to a compound that can combine with a GPR124 receptor to produce or increase a molecular and cellular activity. An agonist may be a ligand that directly binds to the receptor. Alternatively, an agonist may combine with a receptor indirectly by, for example, (a) forming a complex with another molecule or protein that directly binds to the receptor, or (b) otherwise results in the modification of another compound so that the other compound directly binds to the GPR124 receptor.

The term “activate”, and variations thereof refers to any measurable increase in molecular and cellular activity.

The term “antagonist” refers to a compound that can combine with a GPR124 receptor to reduce or inhibit a molecular and cellular activity. An antagonist may be a ligand that directly binds to the receptor. Alternatively, an antagonist may combine with a receptor indirectly by, for example, (a) forming a complex with another molecule or protein that directly binds to the receptor, or (b) otherwise results in the modification of another compound so that the other compound directly binds to the GPR124 receptor.

As used herein, the term “polypeptide” is intended to encompass a singular “polypeptide” as well as plural “polypeptides,” and comprises any chain or chains of two or more amino acid residues linked by peptide bonds. Thus, as used herein, terms including, but not limited to “peptide,” “dipeptide,” “tripeptide,” “protein,” “amino acid chain,” or any other term used to refer to a chain or chains of two or more amino acids, are included in the definition of a “polypeptide,” and the term “polypeptide” can be used instead of or interchangeably with any of these terms. The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids.

### Methods of Preparation

The compounds of the disclosure may be prepared by use of known chemical reactions and procedures. Representative methods for synthesizing compounds of the disclosure are presented below. It is understood that the nature of the substituents required for the desired target compound often determines the preferred method of synthesis. All variable groups of these methods are as described in the generic description if they are not specifically defined below.

### General Procedure

Those having skill in the art will recognize that the starting materials and reaction conditions may be varied, the sequence of the reactions altered, and additional steps employed to produce compounds encompassed by the disclosure, as demonstrated by the following examples. Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Starting materials can be obtained from commercial sources or prepared by well-established literature methods known to those of ordinary skill in the art. The reactions are performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure.

In some cases, protection of certain reactive functionalities may be necessary to achieve some of the above transformations. In general, the need for such protecting groups as well as the conditions necessary to attach and remove such groups will be apparent to those skilled in the art of organic synthesis. An authoritative account describing the many alternatives to the trained practitioner are J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E Gross and J. Meienhofer), Academic Press, London and New York 1981, in “Methoden der organischen Chemie”, Houben-Weyl, 4.sup.th edition, Vol. 15/I, Georg Theme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann “Chemie der Kohlenhydrate: Monosaccharide and Derivate”, Georg Theme Verlag, Stuttgart 1974. The protecting groups may be removed at a convenient subsequent stage using methods known from the art.

Chemical names in this document were generated using Chemdraw Ultra Version 10.0 or Version 12.0, commercially available from CambridgeSoft.

DMSO (dimethyl sulfoxide), Trypsin-EDTA (0.25%), HEPES, NaHCO₃, KCl, CaCl₂, MgSO₄, glucose, NaOH and Triton X-100 were all purchased from Sigma-Aldrich (St. Louis, Mo.). NaCl, HCl (hydrochloric acid), and EDTA were purchased from Fisher Scientific (Santa Clara, Calif.). NaH₂PO₄ was purchased from JT Baker Analytical (Batavia, Ill.). [³H]-CP55,940 ((−)cis-3R-[2-Hydroxy-4-(1,1-dimethyl-[2,3,4,4-³H₄]-heptyl)phenyl]-trans-4R-3(hydroxypropyl)-1R-cyclohexanol) (0.54 mCi/ml), and CP55,940, Δ⁹-THC, was provided by the National Institute of Drug Abuse Drug Supply Program (RTI, Research Triangle Park, N.C.). WIN55,212-2 was from Cayman Chemicals (Ann Arbor, Mich.). HU-210 was purchased from Tocris Bioscience (Ellisville, Mo.). All drugs were dissolved in DMSO, unless otherwise stated and stored at −20° C. until used for experiments.

### EXAMPLES

The preparation and utility of the compounds of the disclosure is illustrated further by the following examples, which are not to be construed as limiting the disclosure in scope or spirit to the specific procedures and compounds described in them. In all cases, unless otherwise specified, the column chromatography is performed using a silica gel solid phase.

### Example 1

### Synthesis of (9-ethyl-9H-carbazol-3-yl)(4-methylnaphthalen-1-yl)methanone, ST-34

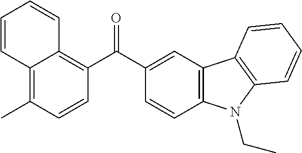 [SMILES: CCn1c2ccccc2c2cc(C(=O)c3ccc(C)c4ccccc34)ccc21]

### Synthesis of 9-ethyl-9H-carbazole (compound 1.1)

A mixture of carbazole (10 g, 59.80 mmol), ethyl bromide (6.65 mL, 89.75 mmol), and powdered NaOH (4 g, 100 mmol) in dry acetone (100 mL) was refluxed for 16 h under nitrogen. The organic solvents were evaporated in vacuo. The obtained residue was diluted with water (50 mL) and extracted into tert-butyl methyl ether (100 mL). The organic layer was washed with water, brine, dried (MgSO4), filtered, and evaporated in vacuo. The obtained residue was crystallized from ethanol. Yield: 8.62 g (74%); mp 70-71° C.

### Synthesis of (9-ethyl-9H-carbazol-3-yl)(4-methylnaphthalen-1-yl)methanone. ST-34

Under argon atmosphere, AlCl₃ (309 mg, 2.32 mmol) was added to a solution of carbazole 1.1 (426 mg, 2.18 mmol) in dry benzene (30) mL, and the obtained solution was placed in an ice-water bath for 20 min. 4-methyl-1-naphthoyl chloride (Huffman et al., Bioorganic & Medicinal Chemistry 13:89 (2005)) (487.87 mg, 2.62 mmol) was added dropwise via a syringe to the solution, and the reaction mixture was then allowed to warm to room temperature and stirred for 16 h. The reaction mixture was cooled on an ice-water bath then poured onto a mixture of ice and concentrated NaOH and extracted with diethyl ether. The organic phase was washed with saturated aqueous sodium bicarbonate, brine, dried (MgSO₄), filtered and evaporated in vacuo. The obtained residue was purified by column chromatography on silica gel eluting with ethyl acetate/heptanes in different proportions to provide example 1 as a yellow glass. Yield: 427 mg (54%). LC/MS m/s [M+H]⁺ 364.07

### Example 2

### Synthesis of 5-ethyl-7-methoxy-2-[(4-methylnaphthalen-1-yl)carbonyl]-1H,2H,3H,4H,5H-pyrido[4,3-b]indole; ST-33

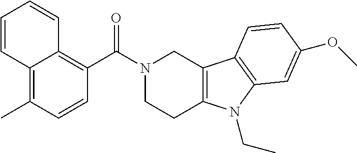 [SMILES: CCn1c2c(c3ccc(OC)cc31)CN(C(=O)c1ccc(C)c3ccccc13)CC2]

### 2.1. Synthesis of ethyl 7-methoxy-3,4-dihydro-1H-pyrido[4,3-b]indole-2(5H)-carboxylate (compound 2.1)

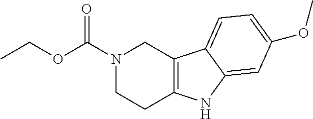 [SMILES: CCOC(=O)N1CCc2[nH]c3cc(OC)ccc3c2C1]

Title compound was prepared by heating phenylhydrazine hydrochloride (4.750 g, 27.2 mmol) and 1-carbethoxy-4-piperidone (5.588 g, 32.64 mmol) in anhydrous ethanol (150 mL) at reflux for 16 h. The solvent was evaporated in vacuo, and the obtained residue was purified by silica gel chromatography using ethyl acetate/heptanes in different proportions to afford the title compound as a white solid. Yield 4.52 g (61%).

### Synthesis of ethyl 5-ethyl-7-methoxy-3,4-dihydro-1H-pyrido[4,3-b]indole-2(5H)-carboxylate (compound 2.2)

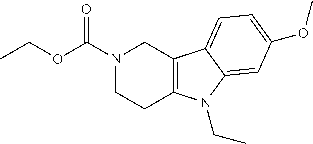 [SMILES: CCOC(=O)N1CCc2c(c3ccc(OC)cc3n2CC)C1]

Sodium hydride (131 mg, 3.28 mmol) in the form of a 60% dispersion in oil was washed with pentanes (25 mL) on a glass filter and added in small portions to a solution of 2.1 (0.5 g, 1.82 mmol) in DMF at 0° C. under N₂. Then, ethyl bromide (2.73 mmol) was added at 0° C. and the mixture was stirred at room temperature for 1 h. The reaction mixture was quenched with saturated aqueous ammonium chloride (3 mL) on an ice-water bath, and extracted with ethyl acetates (150 mL). The organic phase was washed with saturated aqueous sodium bicarbonate (50 mL×2), brine (50 mL), dried (MgSO₄), filtered and evaporated in vacuo. The obtained residue was purified by column chromatography on silica gel using ethyl acetate/heptanes in different proportions to afford the title compound (460 mg, 83%) as a yellowish glass.

### Synthesis of 5-ethyl-7-methoxy-2,3,4,5-tetrahydro-1H-pyrido[4,3-b]indole (compound 2.3)

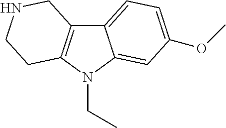 [SMILES: CCn1c2c(c3ccc(OC)cc31)CNCC2]

Solid KOH 4166 mg, 74.39 mmol) was added to a solution of carbethoxyindole 2.2 (460 mg, 1.69 mmol) in a mixture of ethanol (80 mL) and water (10 mL). The resulting solution was heated at reflux under N₂ for 48 h. The obtained solution was concentrated in vacuo to remove ethanol, diluted with saturated aqueous sodium bicarbonate (50 mL) and extracted with ethyl acetate (150 mL). The organic phase was washed with saturated aqueous sodium bicarbonate (50 mL×2), brine (50 mL), dried (MgSO₄), filtered and evaporated in vacuo. The obtained residue was purified on a Biotage® KP-NH cartridge (amino-modified silica gel) using heptanes/ethyl acetate in different proportions to afford the title compound as a yellowish oil (free base form). Yield: 273 mg (70%).

### 2-benzoyl-7-methoxy-5-pentyl-1H,2H,3H,4H,5H-pyrido[4,3-b]indole (Example 2, ST-33)

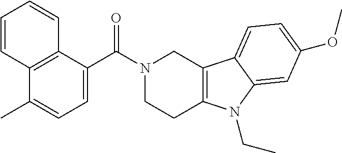 [SMILES: CCn1c2c(c3ccc(OC)cc31)CN(C(=O)c1ccc(C)c3ccccc13)CC2]

Free-base form of amine 23 (273 mg, 1.19 mmol) was suspended in anhydrous DCM (120 mL) under nitrogen, and the obtained suspension was cooled with ice-cold water. DIPEA (429 mmol) was added to the solution, followed by and 4-methyl-1-naphthoyl chloride (364 mg, 1.78 mmol). The flask was removed from the ice bath, and the reaction mixture was stirred for 3 h. After concentration, the residue was purified by column chromatography on silica gel, eluting with EtOAc/heptanes in different proportions to afford example 2 as a pale greenish solid. Yield: 261 mg (55%); mp 209-210° C. LC/MS m/s [M+H]⁺ 399.15

### Example 3

### Cell Culture

All cell lines were grown at 5% CO₂ and 37° C. in cell culture growth media consisting of DMEM+GlutaMAX™-I (Gibco, Carlsbad, Calif.) supplemented with HEPES (10 mM), NaHCO₃ (5 mM), penicillin (100 U/ml)/streptomycin (00 μg/ml) and 10% FBS (heat-inactivated at 65° C. for 30 min) in 10 cm Falcon dishes (BD Biosciences, San Jose, Calif.). Cell maintenance consisted of media changes approximately every 3 days and when cells became 90% confluent cells were trypsinized (1× 0.25% Trypsin-EDTA, GIbco, Carlsbad, Calif.), resuspended in growth media and re-plated in cell culture dishes at a 1:10 dilution.

### Generation of Stable HEK293 Cell Lines.

Stable CB₁ and CB₂ expressing HEK293 cell lines were generated using plasmids containing full coding region of mouse CB₁ and CB₂.

Fragments were amplified from total RNA of cell lines by reverse transcriptase-polymerase chain reaction (RT-PCR). Sequence was confirmed and the fragment was cloned into the EcoRI site of the pIRES2-eGFP-express vector. Cells were grown to approximately 80% confluence in 10 cm cell culture dishes, transfected with the cDNA pIRES-eGFP-vector containing the human CB₁ or CB₂ receptor using LipofectAMINE™ 2000 reagent in the serum-free media Opti-MEM 1 according to the manufacturer's description. Cells were subject to FACS sorting 48 his after transfection and single cell sorted based on dsRed expression into 96-well plates. Of the dsRed expressing positive clones, 3 were validated for CB₁ and CB₂ protein expression by radioligand binding analysis (methods discussed below).

### Example 4

### Radioligand Binding

### Membrane Protein Preparations for Radioligand Binding.

Cells were grown to 90% confluence, rinsed twice with 1× PBS and stored at −80° C. until further use. To prepare crude cellular membrane fractions, dishes were removed from −80° C. and thawed at room temperature for 5 min. Once thawed, cells were lysed with ice-cold homogenization buffer (50 mM Tris, 1 mM EDTA, 3 mM MgCl₂) and gently scraped from the dish. Total cell lysates from 3-5 dishes were collected on ice in 3 ml of ice-cold homogenization buffer Cells were homogenized at 6,500 rpm (PRO Scientific, Oxford, Conn.) twice for 10 sec and centrifuged at 11,500 rpm for 20 min at 4° C. Supernatants were discarded and cell pellets were resuspended in homogenization buffer and tris rated 10 times in ice-cold homogenization buffer. Crude cellular membrane homogenates were stored at −80° C. until further use. On the day of experiments, frozen homogenates were thawed at room temperature, gently triturated 10 times and subsequently dounce homogenized in a 7 ml glass tissue grinder (Wheaton Science Products, Millville, N.J.) by 5 strokes while on ice. The DC protein assay (BioRad, Hercules, Calif.) was utilized as instructed by the manufacturer to determine protein concentrations and BSA in homogenization buffer (see radioligand binding assay methods) was used for protein standards.

### Radioligand Binding Assay.

Due to the lipophillic nature of the tested compounds, all experiments were performed in silanized glass test tubes (Alltech, Deerfield, Ill.) with silanized pipette tips (VWR Scientific, Brisbane, Calif.) to reduce the loss of ligands. While on ice, the reaction components were added to the test tubes in the following order 50 μl of non-radiolabeled ligand, 50 μl of radioligand and 100 μl of protein (50 μg) for a total reaction volume of 200 μl. The reactions were initiated with the addition of protein. Reactions did not contain greater than 0.1% DMSO. However, due to solubility limitations of some of the tested compounds, greater DMSO concentrations were used but controlled for in each experiment with the appropriate DMSO control. All components were prepared in binding buffer (50 mM Tris base, 1 mM EDTA, 3 mM MgCl₂, 1 mg/ml BSA, pH=7.4) Once reactions were initiated, tubes were covered with parafilm and incubated in a water bath held at 30° C. with mild agitation for 1 hour. Reactions were stowed by adding ice-cold binding buffer under rapid filtration using the Brandel harvester (Brandel, Gaithersburg, Md.) and collected on Whatman GFB filter strips (Brandel, Gaithersburg Md.) that had been incubated in binding buffer for 1 h at mom temperature. Filters were immediately transferred to 7 ml glass scintillation vials (VWR Scientific, Brisbane, Calif.) using the Brandel Manual Deposit (Brandel, Gaithersburg, Md.) and 5 ml scintillation fluid (National Diagnostics, Atlanta Ga.; Ecoscint XR) was added to each scintillation vial. Samples were rapidly vortexed for 10 sec, followed by 3 hour incubation at room temperature prior to obtaining radioactive counts in the scintillation counter (PerkinElmer, Boston Mass.). For radioligand saturation curves, [³H]-CP95440 concentrations varied while using a constant saturating dose of a high-affinity non-radiolabeled ligand to determine non-specific binding. In radioligand competition experiments, [³H]-CP95440 was held at the calculated K_{d}, ˜1 nM, and the competing ligand concentrations varied. For competition binding assays, specific binding was calculated by subtracting the average dpm (disintegrations per minute, dpm) if non-specific points from the individual dpm values for each total binding point and expressed as either fmol/mg for saturation analyses or as a percent of total radioligand binding in competition curves. All experiments were performed in duplicate or triplicate, at a minimum of three different experiments.

### Example 5

### Cell Viability Assay

Cells were plated media supplemented with 10% serum in 96-well plates (10⁴ cells per wellI; 0.1 ml per well). Once they reached ˜70% confluence, they were rinsed with PBS and kept for an additional 24 his in media supplemented with 1% serum, at which time drugs or vehicle (DMSO, 0.1%, prepared in 10 μl serum-free media) were directly added to each well. After 3 days, cell viability was assessed using the Cell Proliferation Reagent WST-1 (Roche, Indianapolis, Ind.). Briefly, WST-1 reagent (10 μl) was added to each well for 3 hrs at 37° C. with 5% CO₂ and WST-1 products were read at 450 nm using Packard SpectraCount™.

### Example 6

### Quantitative RT-PCR

RNA was extracted using PrefectPure RNA Cultured Cell Kit (5 prime) Real-time quantitative PCR assays were performed using the Brilliant® II QRT-PCR Master Mix, 1-Step kit (Stratagem) and probes were obtained from the Universal Probe Library Set (Roche Applied Science). The following sense/antisense primers and probes were used: human CB₁: 5′-TGTCTGTCTGCACACCTTGAA-3′ and 5′-CATCTGCACAT GACAGAGAGG-3′, probe #40; human CB₂: 5′-TGGGAGAGGACAGAAAACAACT-3′ and 5′-GAGCTTGTCTAGAAGGCTTTGG-3′, probe #24; human GPR124 5′-GGCTCCT TCCTGGGACTG-3′ and 5′-GCACTGTGCTGATGATGTTGT-3′-probe #67; mouse GPR124 5′-GTCCCTGTTGGAGAAGTTGG-3′ and 5′-AGCGMTAGCTCTCTC CCAGA-3′, probe #1. Universal ProbeLibrary Human HPRT Gene Assays (Roche Applied Science) were used as references in dual color qPCR reactions Amplifications were run in a Mx3000P™ Real-Time PCR System (Stratgene).

### Example 7

### Data Analysis

All data were analyzed using the GraphPad PRISM® 4.02 program (GraphPad Software, San Diego, Calif.). Data from radioligand binding experiments were calculated as follows: the average dpm values of the non-specific points were subtracted from each individual total dpm values. For saturation analyses, the calculated specific binding values were expressed as fmol/mg and graphed against the free concentrations of [³H]-CP55940. All radioligand competition binding values were normalized by expressing values as a percent of the maximal amount of radioligand displaced by a non-radiolabeled compound in each cell line. All data is represented as a mean±SEM. Statistical analyses were performed using GraphPad PRISM® 4.02.

### Example 8

### Design of a Genetic Approach to Identify the Gene Encoding for AAI Receptors

### 1: Select Cells Lacking CB₁/CB₂ Receptors:

The expression level of CB₁ and CB₂ mRNA is determined by qPCR in eight human cell lines and found three, T98 g, MDA231 and sknmc cells, that lack CB₁ and CB₂ mRNA (Table 1).

TABLE 1

| human cell lines | CB1, ΔCt | CB2, ΔCt |
|---|---|---|
| T98G | no ct | no ct |
| MDA231 | no ct | no ct |
| sknmc | no ct | no ct |
| SW480 | 8.29 | no ct |
| SF767 | 11.11 | no ct |
| HT29 | 13.41 | no ct |
| U87 | 7.73 | no ct |
| hek | 10.13 | no ct |
| U3t3 cDNA | −5.22 | no ct |
| HL60 cDNA | 6.37 | −3.49 |

### 2. Determine the Sensitivity Profile of CB₁/CB₂-KO Cells to WIN155212-2:

WIN55212-2 (ST-1 in Table 1) has been shown to kill tumor cells in culture independently of CB₁/CB₂ receptors. Based on this evidence, it was determined if WIN55212-2 differentially kills T98 g, MDA231 and dome cells, thus providing an index of AI receptor functionality in these cells. Also tested is Δ⁹-THC (classic cannabinoid) and CP55940 (non-classical cannabinoid).

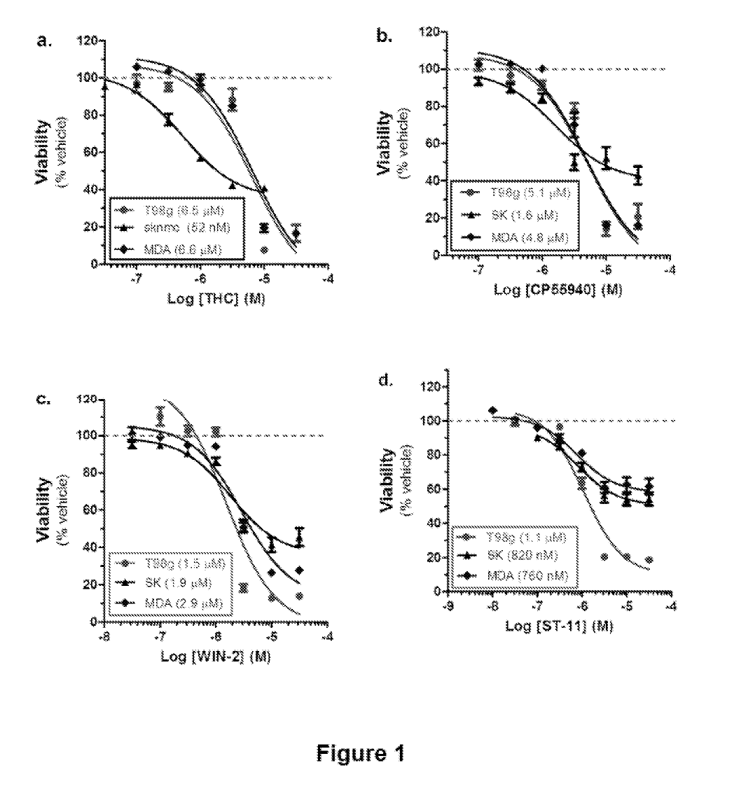

The potency of WIN55212-2 (ST-1) at killing T98 g, MDA231 and sknmc cells was within similar micromolar ranges (1.5-2.9 μM) and exhibited gradual toxic efficacy (T98 g>MDA>sknmc) (FIG. 1). Conversely, both THC and CP55940 exhibited inconsistent potencies and efficacies at killing these cells, suggesting that T98 g, MDA231 and slows cells express AI receptors at different expression levels, and that AI compounds induce the strongest toxic response in the human astrocytoma cell line T98 g.

### 3: SAR Study and Optimization of AAI Compounds:

Because the strongest toxic effect of WIN55212-2 (ST-1) was measured in T98 g cells, these cells are used as readout to study the structure activity relationship (SAR) of AI compounds at inducing cell death. Table 2 provides the chemical structure, potency and efficacy of several AI compounds.

TABLE 2

| Compound ID | Structure | EC₅₀ (μM) |
|---|---|---|
| ST-1 WIN55212-2 | 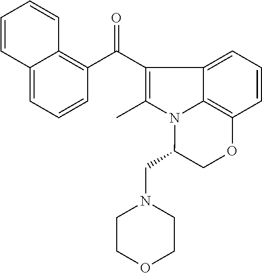 [SMILES: Cc1c(C(=O)c2cccc3ccccc23)c2cccc3c2n1[C@@H](CN1CCOCC1)CO3] | 2.1 |
| ST-2 | 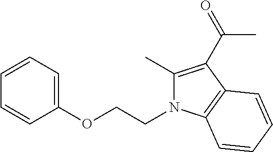 [SMILES: CC(=O)c1c(C)n(CCOc2ccccc2)c2ccccc12] | 21 |
| ST-3 | 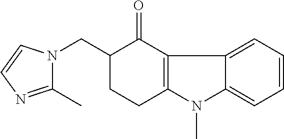 [SMILES: Cc1nccn1CC1CCc2c(c3ccccc3n2C)C1=O] | inactive |
| ST-4 | 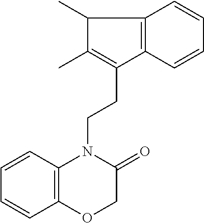 [SMILES: CC1=C(CCN2C(=O)COc3ccccc32)c2ccccc2C1C] | inactive |
| ST-5 | 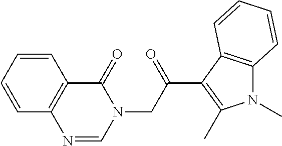 [SMILES: Cc1c(C(=O)Cn2cnc3ccccc3c2=O)c2ccccc2n1C] | inactive |
| ST-6 | 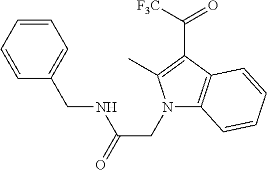 [SMILES: Cc1c(C(=O)C(F)(F)F)c2ccccc2n1CC(=O)NCc1ccccc1] | inactive |
| ST-7 | 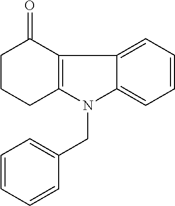 [SMILES: O=C1CCCc2c1c1ccccc1n2Cc1ccccc1] | inactive |
| ST-8 | 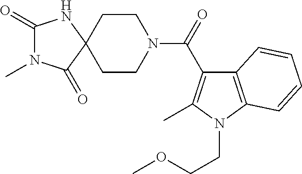 [SMILES: COCCn1c(C)c(C(=O)N2CCC3(CC2)NC(=O)N(C)C3=O)c2ccccc21] | 29 |
| ST-9 | 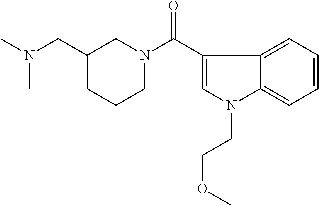 [SMILES: COCCn1cc(C(=O)N2CCCC(CN(C)C)C2)c2ccccc21] | inactive |
| ST-10 | 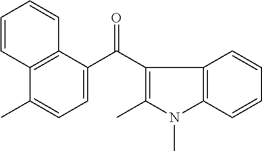 [SMILES: Cc1ccc(C(=O)c2c(C)n(C)c3ccccc23)c2ccccc12] | 2.9 |
| ST-11 | 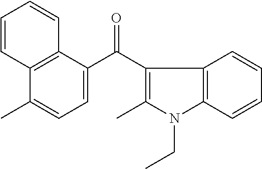 [SMILES: CCn1c(C)c(C(=O)c2ccc(C)c3ccccc23)c2ccccc21] | 1.3 |
| ST-12 | 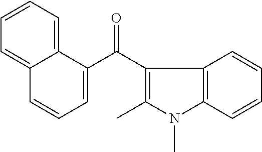 [SMILES: Cc1c(C(=O)c2cccc3ccccc23)c2ccccc2n1C] | 3.1 |
| ST-13 | 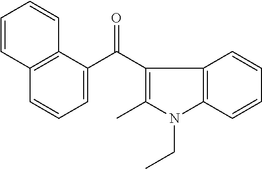 [SMILES: CCn1c(C)c(C(=O)c2cccc3ccccc23)c2ccccc21] | 7.9 |
| ST-14 | 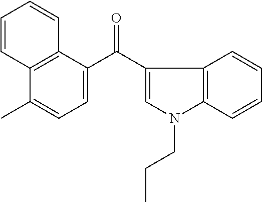 [SMILES: CCCn1cc(C(=O)c2ccc(C)c3ccccc23)c2ccccc21] | inactive |
| ST-15 | 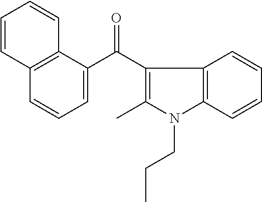 [SMILES: CCCn1c(C)c(C(=O)c2cccc3ccccc23)c2ccccc21] | 1.5 |
| ST-16 | 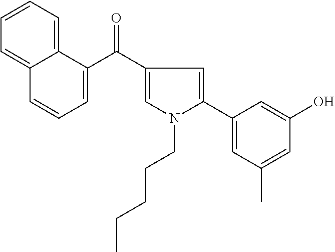 [SMILES: CCCCCn1cc(C(=O)c2cccc3ccccc23)cc1-c1cc(C)cc(O)c1] | inactive |
| ST-17 | 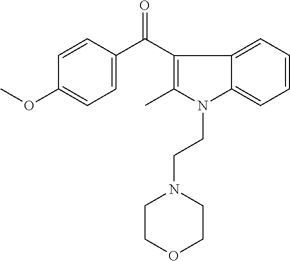 [SMILES: COc1ccc(C(=O)c2c(C)n(CCN3CCOCC3)c3ccccc23)cc1] | 24 |
| ST-18 | 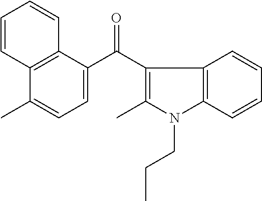 [SMILES: CCCn1c(C)c(C(=O)c2ccc(C)c3ccccc23)c2ccccc21] | 1.9 |
| ST-19 | 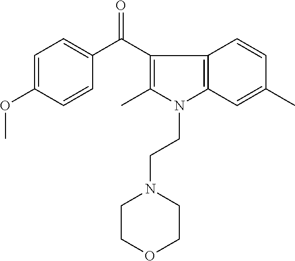 [SMILES: COc1ccc(C(=O)c2c(C)n(CCN3CCOCC3)c3cc(C)ccc23)cc1] | 7.4 |
| ST-20 | 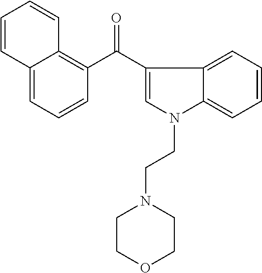 [SMILES: O=C(c1cccc2ccccc12)c1cn(CCN2CCOCC2)c2ccccc12] | 23 |
| ST-21 | 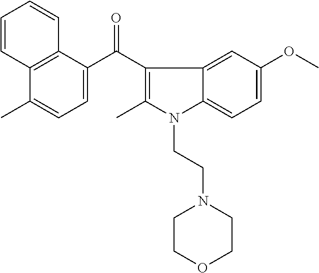 [SMILES: COc1ccc2c(c1)c(C(=O)c1ccc(C)c3ccccc13)c(C)n2CCN1CCOCC1] | 29 |
| ST-22 | 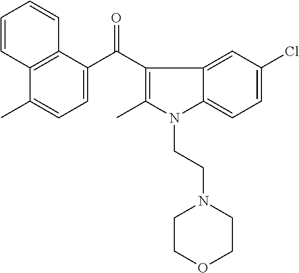 [SMILES: Cc1ccc(C(=O)c2c(C)n(CCN3CCOCC3)c3ccc(Cl)cc23)c2ccccc12] | 5.4 |
| ST-23 | 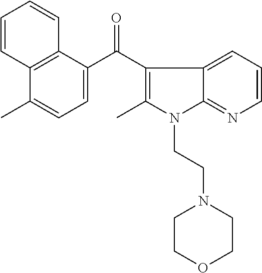 [SMILES: Cc1ccc(C(=O)c2c(C)n(CCN3CCOCC3)c3ncccc23)c2ccccc12] | 3.5 |
| ST-24 | 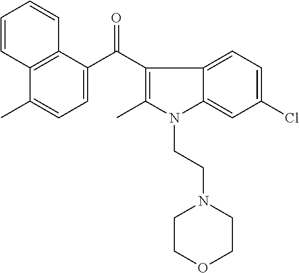 [SMILES: Cc1ccc(C(=O)c2c(C)n(CCN3CCOCC3)c3cc(Cl)ccc23)c2ccccc12] | 1.4 |
| ST-25 | 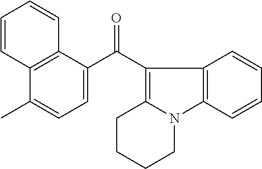 [SMILES: Cc1ccc(C(=O)c2c3n(c4ccccc24)CCCC3)c2ccccc12] | 0.634 |
| ST-26 | 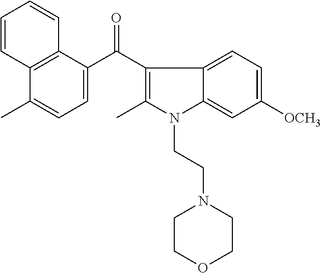 [SMILES: COc1ccc2c(C(=O)c3ccc(C)c4ccccc34)c(C)n(CCN3CCOCC3)c2c1] | 2.5 |
| ST-27 | 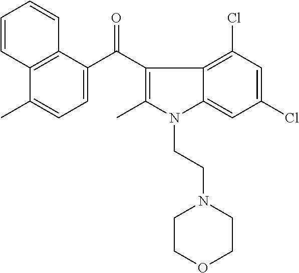 [SMILES: Cc1ccc(C(=O)c2c(C)n(CCN3CCOCC3)c3cc(Cl)cc(Cl)c23)c2ccccc12] | 0.409 |
| ST-28 | 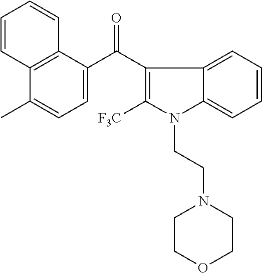 [SMILES: Cc1ccc(C(=O)c2c(C(F)(F)F)n(CCN3CCOCC3)c3ccccc23)c2ccccc12] | 2.7 |
| ST-29 | 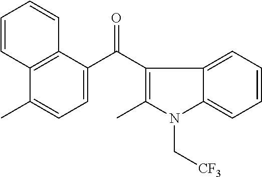 [SMILES: Cc1ccc(C(=O)c2c(C)n(CC(F)(F)F)c3ccccc23)c2ccccc12] | 1.0 |
| ST-30 | 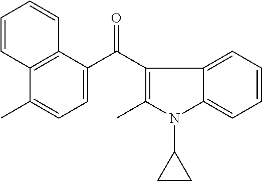 [SMILES: Cc1ccc(C(=O)c2c(C)n(C3CC3)c3ccccc23)c2ccccc12] | 1.0 |
| ST-31 | 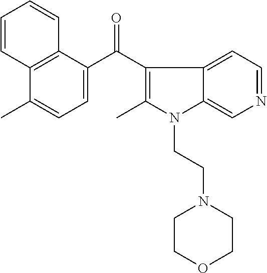 [SMILES: Cc1ccc(C(=O)c2c(C)n(CCN3CCOCC3)c3cnccc23)c2ccccc12] | 20 |
| ST-32 | 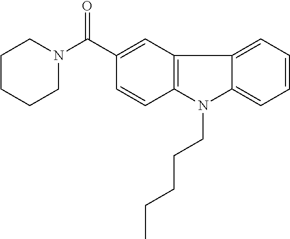 [SMILES: CCCCCn1c2ccccc2c2cc(C(=O)N3CCCCC3)ccc21] | 6.2 |
| ST-33 | 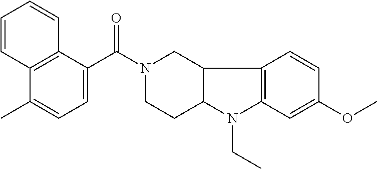 [SMILES: CCN1c2cc(OC)ccc2C2CN(C(=O)c3ccc(C)c4ccccc34)CCC21] | 1.4 |
| ST-34 | 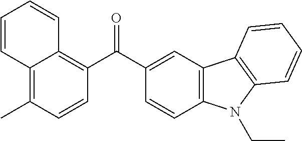 [SMILES: CCn1c2ccccc2c2cc(C(=O)c3ccc(C)c4ccccc34)ccc21] | 0.077 |
| ST-35 | 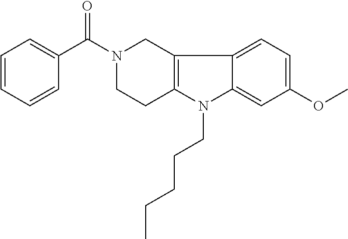 [SMILES: CCCCCn1c2c(c3ccc(OC)cc31)CN(C(=O)c1ccccc1)CC2] | ~30 |
| ST-36 | 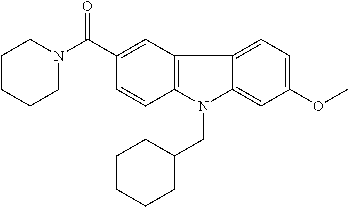 [SMILES: COc1ccc2c3cc(C(=O)N4CCCCC4)ccc3n(CC3CCCCC3)c2c1] | 3.7 |
| ST-37 | 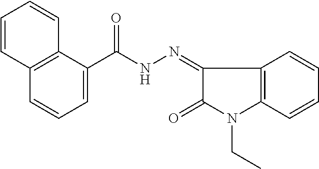 [SMILES: CCN1C(=O)/C(=N\NC(=O)c2cccc3ccccc23)c2ccccc21] | 5.6 |
| ST-40 | 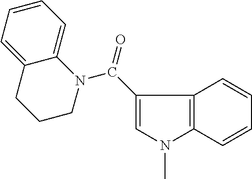 [SMILES: Cn1cc(C(=O)N2CCCc3ccccc32)c2ccccc21] | inactive |
| ST-41 | 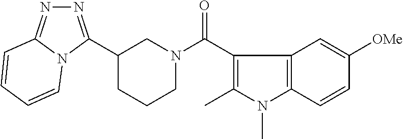 [SMILES: COc1ccc2c(c1)c(C(=O)N1CCCC(c3nnc4ccccn34)C1)c(C)n2C] | inactive |
| ST-42 | 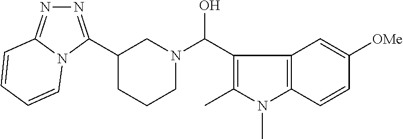 [SMILES: COc1ccc2c(c1)c(C(O)N1CCCC(c3nnc4ccccn34)C1)c(C)n2C] | inactive |
| ST-44 | 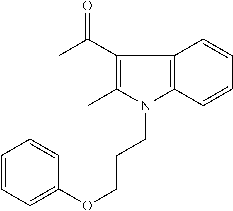 [SMILES: CC(=O)c1c(C)n(CCCOc2ccccc2)c2ccccc12] | >20 |
| ST-45 | 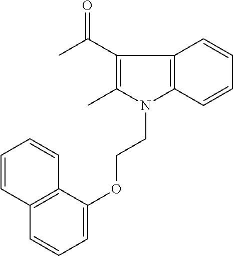 [SMILES: CC(=O)c1c(C)n(CCOc2cccc3ccccc23)c2ccccc12] | 30 |
| ST-46 | 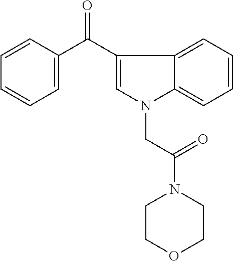 [SMILES: O=C(c1ccccc1)c1cn(CC(=O)N2CCOCC2)c2ccccc12] | inactive |
| ST-47 | 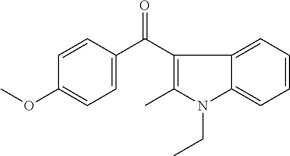 [SMILES: CCn1c(C)c(C(=O)c2ccc(OC)cc2)c2ccccc21] | ~20 |
| ST-48 | 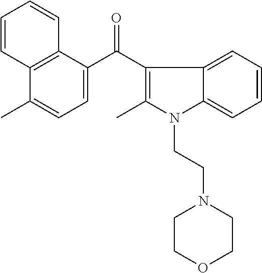 [SMILES: Cc1ccc(C(=O)c2c(C)n(CCN3CCOCC3)c3ccccc23)c2ccccc12] | 6.7 |
| ST-49 | 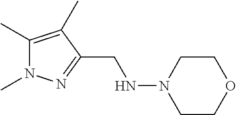 [SMILES: Cc1c(CNN2CCOCC2)nn(C)c1C] | inactive |
| ST-53 | 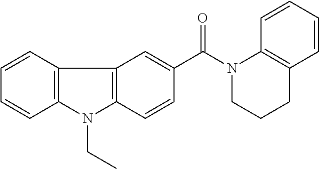 [SMILES: CCn1c2ccccc2c2cc(C(=O)N3CCCc4ccccc43)ccc21] | 0.516 |
| ST-54 | 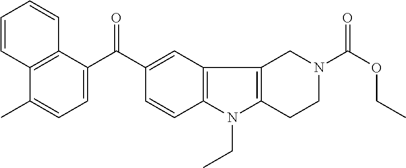 [SMILES: CCOC(=O)N1CCc2c(c3cc(C(=O)c4ccc(C)c5ccccc45)ccc3n2CC)C1] | 6 |
| ST-58 | 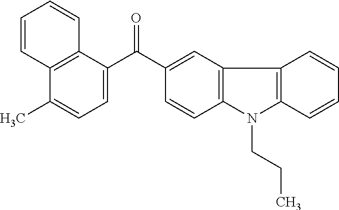 [SMILES: CCCn1c2ccccc2c2cc(C(=O)c3ccc(C)c4ccccc34)ccc21] | inactive |
| ST-59 | 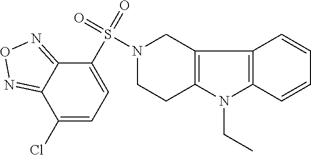 [SMILES: CCn1c2c(c3ccccc31)CN(S(=O)(=O)c1ccc(Cl)c3nonc13)CC2] | inactive |
| ST-60 | 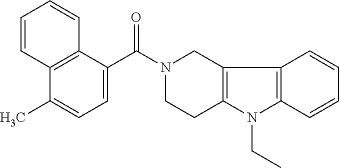 [SMILES: CCn1c2c(c3ccccc31)CN(C(=O)c1ccc(C)c3ccccc13)CC2] | 3 |
| ST-62 | 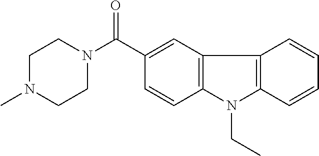 [SMILES: CCn1c2ccccc2c2cc(C(=O)N3CCN(C)CC3)ccc21] | inactive |
| ST-63 | 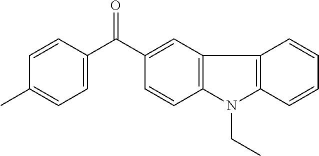 [SMILES: CCn1c2ccccc2c2cc(C(=O)c3ccc(C)cc3)ccc21] | 0.715 |
| ST-64 | 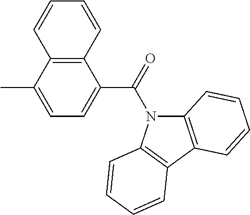 [SMILES: Cc1ccc(C(=O)n2c3ccccc3c3ccccc32)c2ccccc12] | 1.87 |

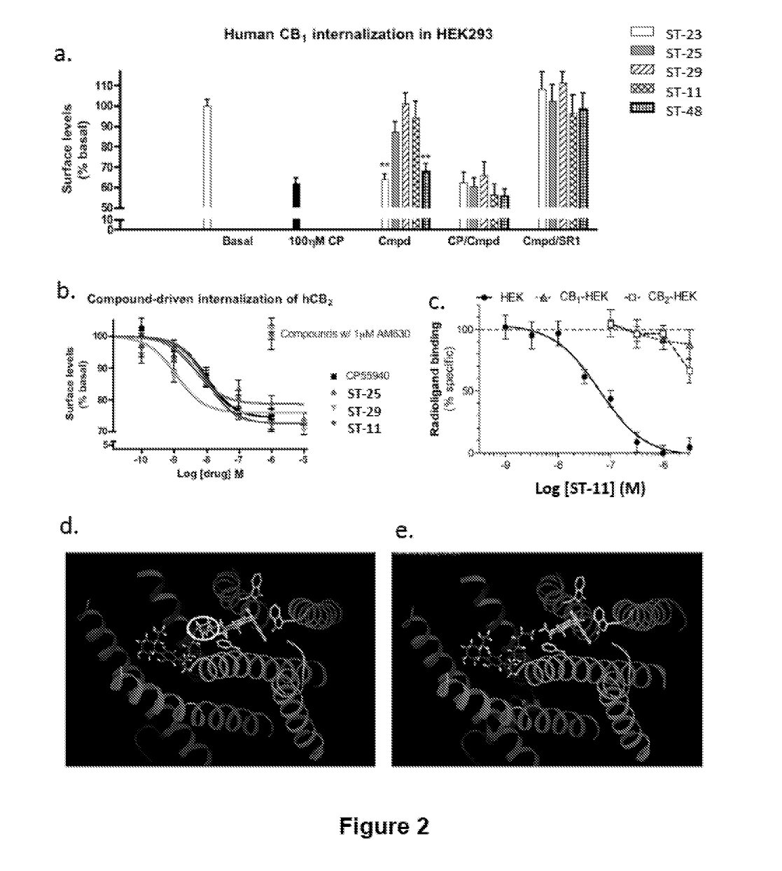

Based on these results and the chemical characteristics of these compounds, we selected several ST compounds, ST-11, ST-23, ST-25, ST-29 and ST-48, and tested their ability to cross-activate human CB₁ and CB₂ receptors, initially by measuring HA-tagged receptor internalization. FIG. 2a shows that ST-23 and ST-48 engage CB₁ receptors and FIG. 2b shows that ST-11, ST-29 and ST-48 engage CB₂ receptors. FIG. 2c ST-11 competes for [3H]-WIN55212-2 in HEK293 cells (circles), but not for [³H]-CP55940 binding at CB₁ (triangle) and CB₂ (squares). FIGS. 2d and 2e illustrate the co-docking of CP55940 and either WIN55212-2 (d) or ST-11 (e) in hCB₁ receptors.

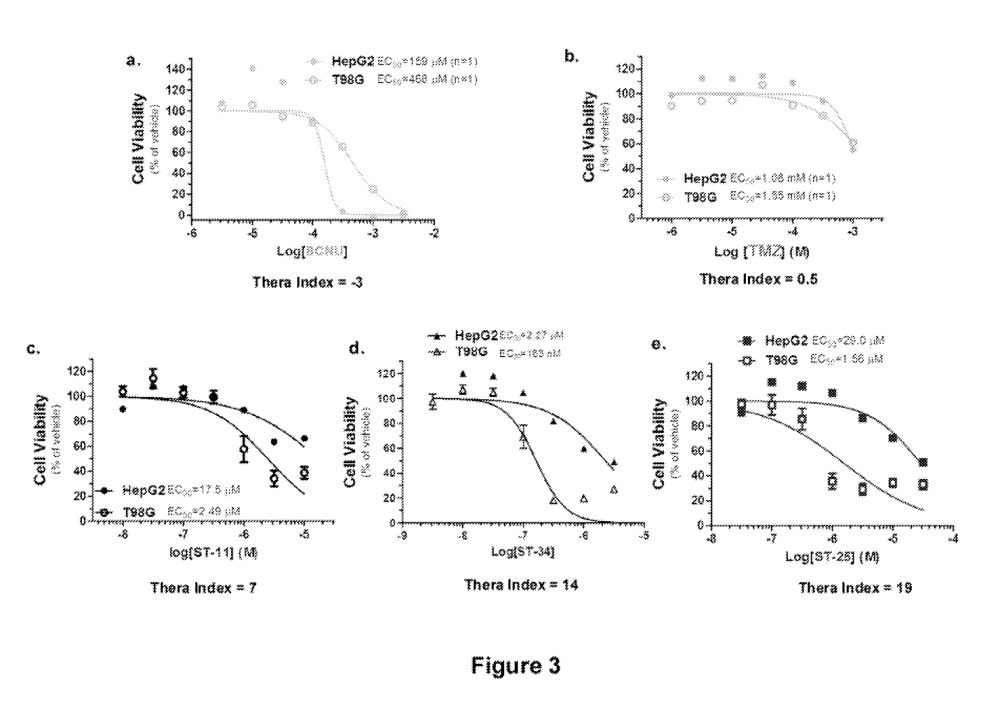

Based on these results, the study was focused on the mechanism of action of ST compounds by using the prototypical scaffolds ST-11, ST-25 and ST-34. FIG. 3 shows the potency of standard care therapeutics (BCNU and temozolamide, TMZ) as compared to ST-11, ST-25 and ST-34 when tested in human T98 g cells and HepG2 cells, providing the in vitro therapeutic index of these compounds. These results indicate that ST-compounds are likely to exhibit lower toxic side effects compared to standard care.

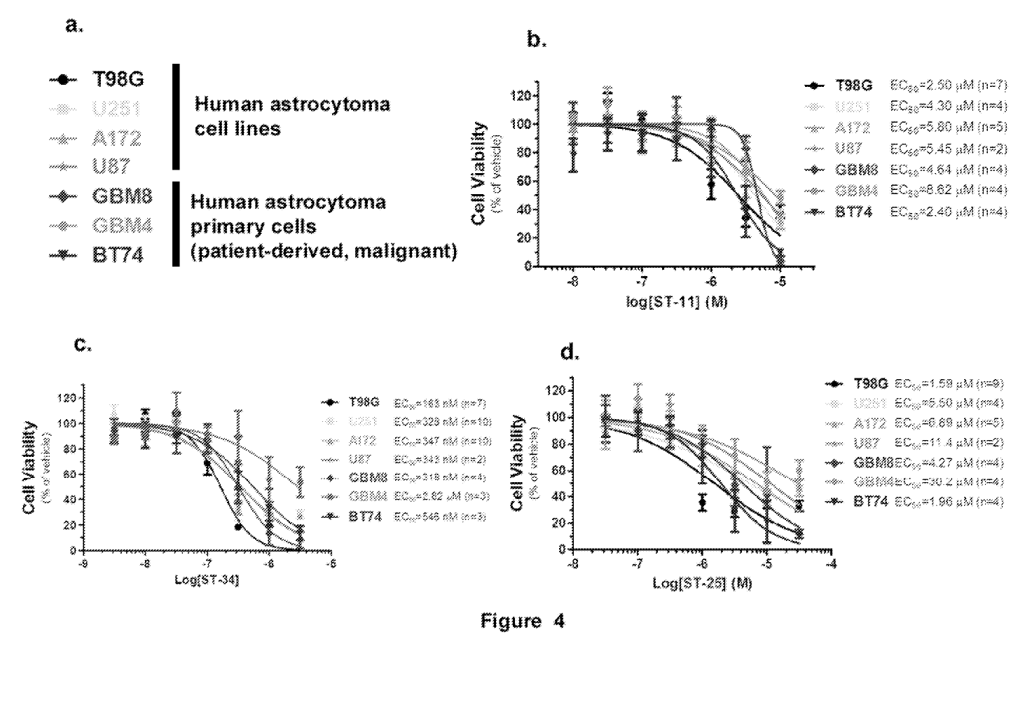

GBM tumors are heterogenous in nature, in the genetic mutations they carry and their sensitivity to standard care. FIG. 4 shows the potency of ST-11, ST-25 and ST-34 in human astrocytomas cell lines and astrocytoma cells derived from patients, suggesting that these compound have a broad array of cells they target, including cells that resist standard care treatment by TMZ (i.e. T98 g and BT72 cells).

### 4: Identification of Mile Candidates for AI Receptors:

Because evidence suggests that AI receptors couple to G proteins, gene array analysis of T98 g, MDA231 and shone cells was performed focusing to mRNA that encode for GPCRs. Gene arrays were generated by pooling total mRNA extracted from three independent cultures for each cell line. AU three cell lines expressed 39 GPCR mRNAs in common. These 39 genes represent gene candidates:

| | | | | | | |
|---|---|---|---|---|---|---|
| GPR1 | GPR126 | GPR18 | GPR25 | GPR45 | GPR124 | GPRS5 |
| GPR116 | GPR135 | GPR19 | GPR37 | GPR50 | GPR64 | GPR97 |
| GPR12 | GPR144 | GPR20 | GPR39 | GPR52 | GPR65 | |
| GPR124 | GPR17 | GPR21 | GPR4 | GPR56 | GPR68 | |
| GPR125 | GPR173 | GPR22 | GPR44 | GPR6 | GPR75 | |

### 5: Identification of GPR124 as an AI Receptor:

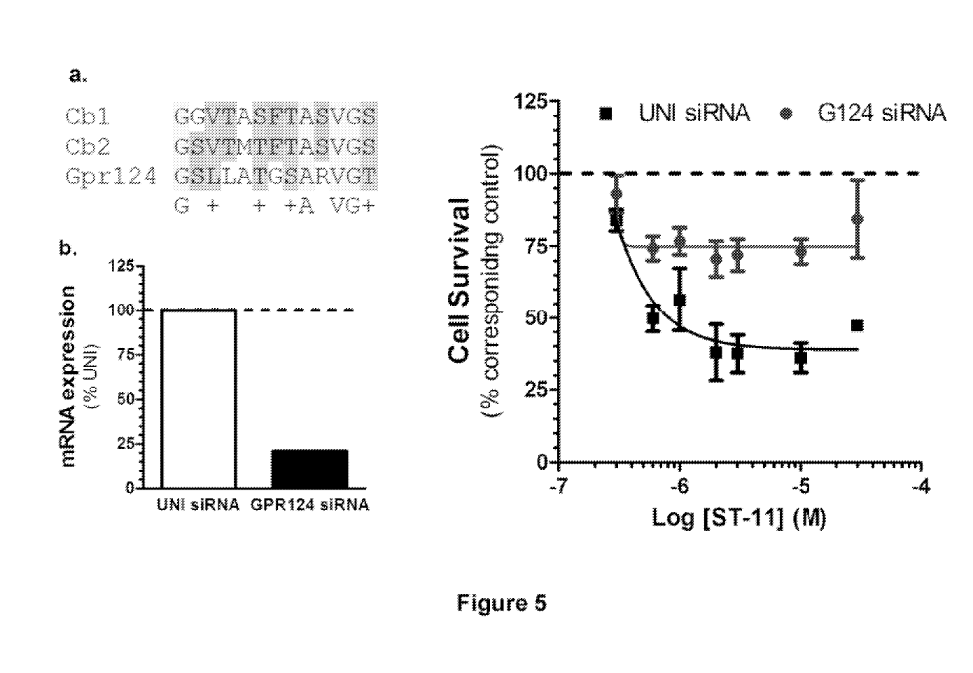

To determine which of the gene candidate encodes for AI receptors, an siRNA approach is used to knock down each candidate individually to determine which one prevents the toxic effect of ST-11 and thus encodes for AI receptors. The prediction is that knocking down AI receptors prevents the cell death induced by ST-11. T98 g cells were treated with siRNA targeting each of the 39 GPCR gene candidates individually. The first experiment was designed to knock down each GPCR candidate liming a mixture of 3 siRNAs, and thus maximize genetic targeting efficacy. Thus, cells were incubated with siRNA mixtures and then treated with ST-11 (1 μM) and cell viability measured after three days. Only one mixture of siRNAs (that targeting GPR124) decreased the toxicity of ST-11. To validate this result, the mixture of 3 siRNA targeting GPR124 was deconvulted by testing each siRNA separately, measuring their respective efficacy at knocking down GPR124 mRNA by qPCR. Two of the three siRNAs knocked-down GPR124 expression by more than 80% over 4 days (FIG. 5b). FIG. 5c shows the results obtained with one of these siRNA, demonstrating that the ST-11-induced killing of T98 g cells is reduced when GPR124 mRNA expression is concomitantly reduced. The killing effect induced by ST-11 is mediated by GPR124, an AI receptor.

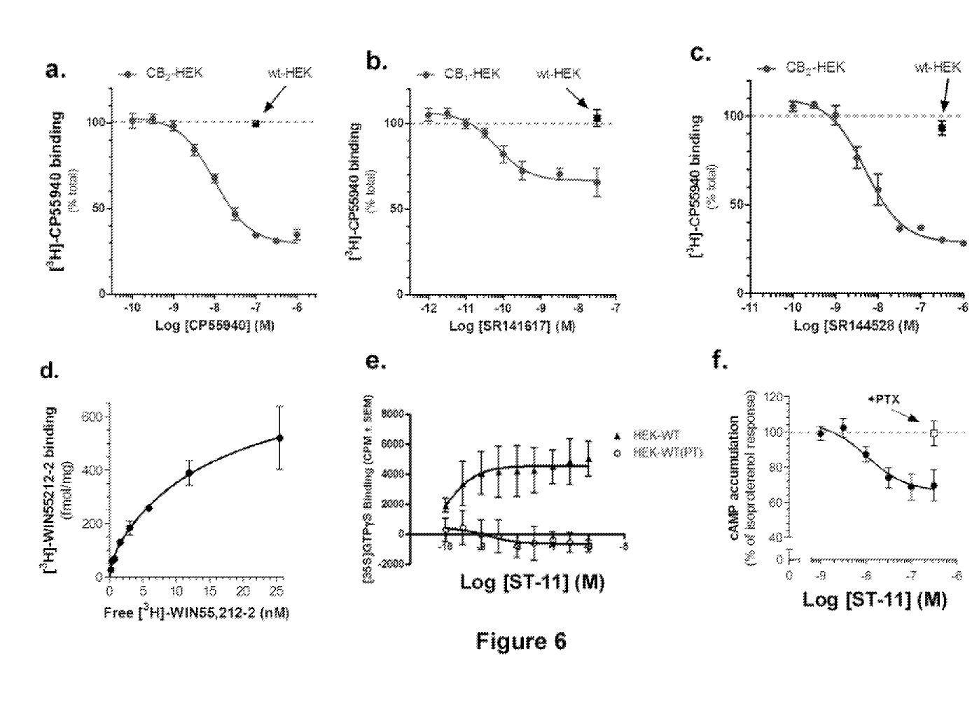

When comparing the overall amino acid sequence of GPR124 to that of CB₁ and CB₂ receptors no significant homology was found. However, when focusing the comparison to the amino acid sequence encoding for the third transmembrane domains of GPR124 and CB₂ receptors, 84% homology was found (FIG. 5a). Site directed mutagenesis studies show that ST-1 interacts directly with the third transmembrane domain of CB₂ receptors. Therefore, it is likely that ST-1, ST-11, and other AI compounds interact directly with the third transmembrane domain of GPR124. To determine the signaling mechanism of AI receptors, effect of ST-11 in HEX293 cells, which express GPR124 endogenously and commonly used to study GPCR pharmacology and signaling pathway, was tested. FIG. 6 a-c shows that HEK293 cells do not express CB₁ and CB₂ receptors as measured by radioligand binding, but express AI receptors as indicated by abundant [³H]-WIN55212-2 binding (d) and activation of GTP□S binding (e) and inhibition of cAMP production by ST-11 (f), suggesting that AI receptor couple to Gi/o proteins.

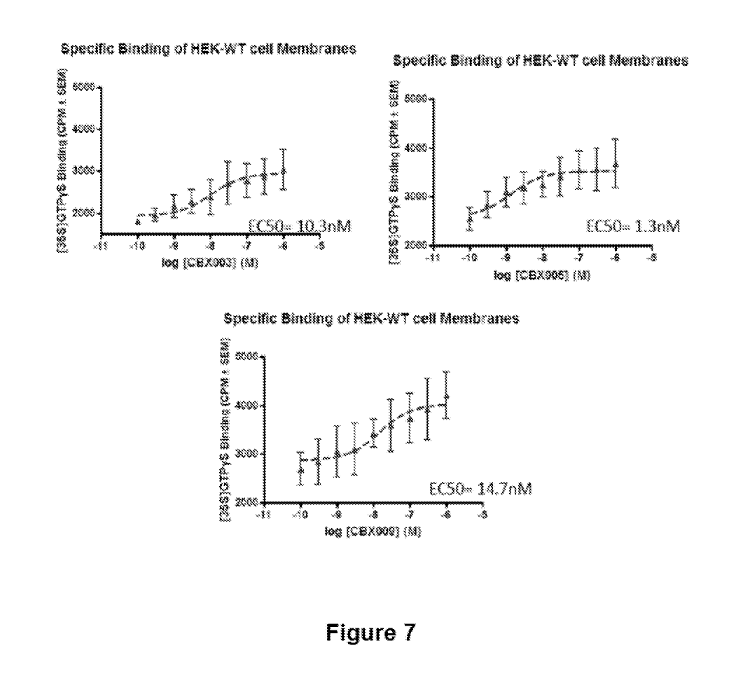

FIG. 7 shows that ST-23, ST-25 and ST-29 also increase GTP□S binding in HEK293 cell homogenates, indicating that these compounds act as agonists at AI receptors.

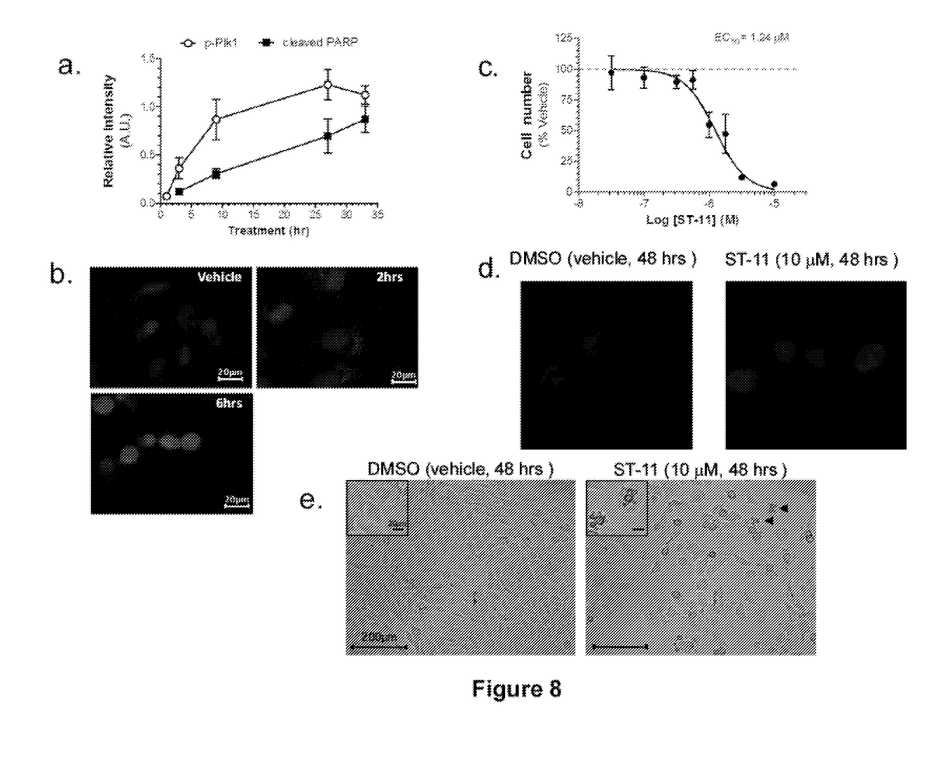

To determine the signaling pathway activated by AI receptors, T98 g cells were treated and changes in select kinase and mediatior of cell death were measured. FIG. 8 shows that ST-11 induces the activation of PLK-1 and cleaves PARP (a) and caspase 3 activation (b) within hours, which is followed by cell death as measured by reduction in cell number (c), nuclear fragmentation (d) and cell blebbing after 48 hrs.

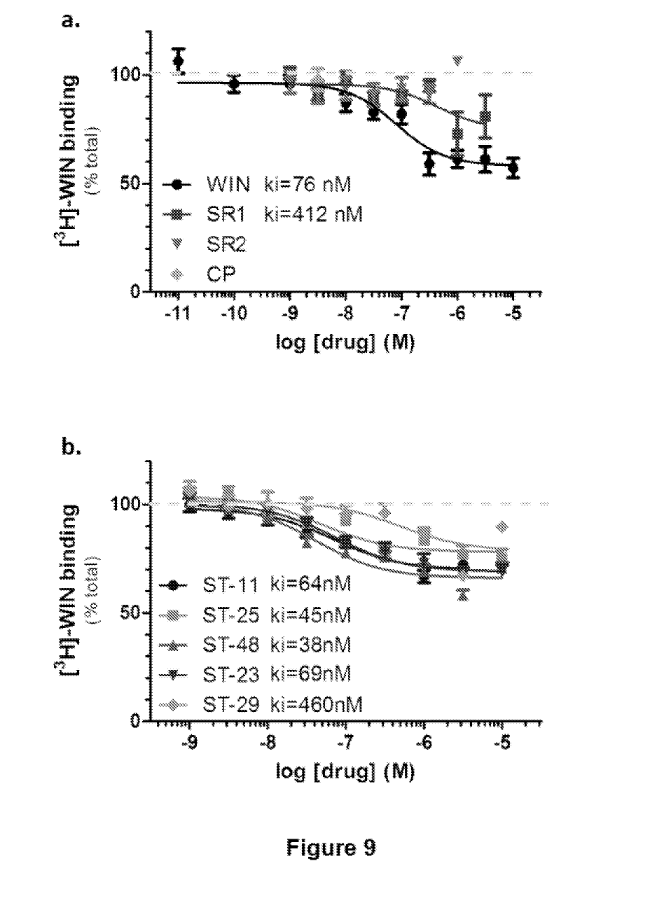

To further study how ST compounds affect astrocytoma cell biology, a mouse astrocytoma cell line that expresses AI receptor endogenously was identified. FIG. 9 shows that DBT cells, a mouse astrocytoma cell line, does not express CB₁ and CB₂ receptors as measured by radioligand binding (a), yet likely express AI receptors as measured by radioligand binding competition with ST compounds.

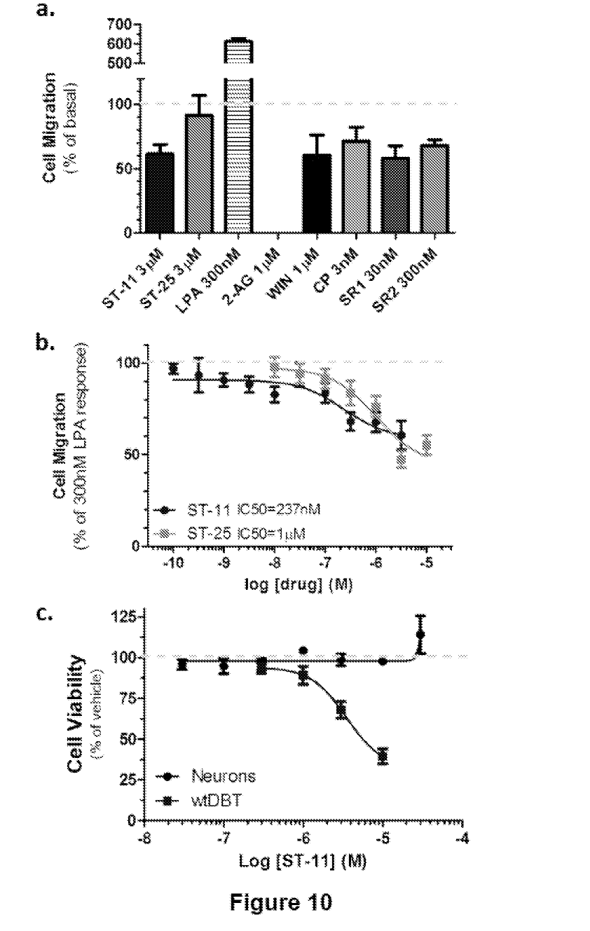

Cannabinoid receptors are known to regulate cell migration. FIG. 10 shows that ST-compounds do not stimulate cell migration (a), yet inhibit DBT cell migration stimulated by LPA (b). ST-11 kills DBT cells but not mouse neurons in primary culture (c).

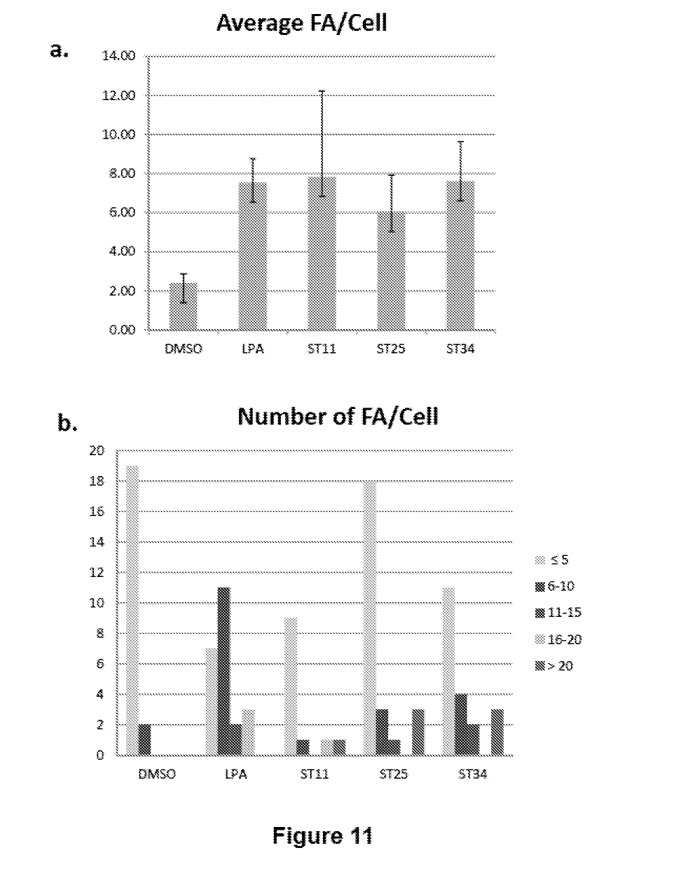

To determine the mechanism of action of ST-compounds on cell migration, the cells in culture were tested with these compounds for their ability to attach to extracellular substrasts. FIG. 11 shows that ST-compounds, similarly to the chemoattactant LPA, increase the number of focal adhesion in the human astrocytoma cell line U87MG cells.

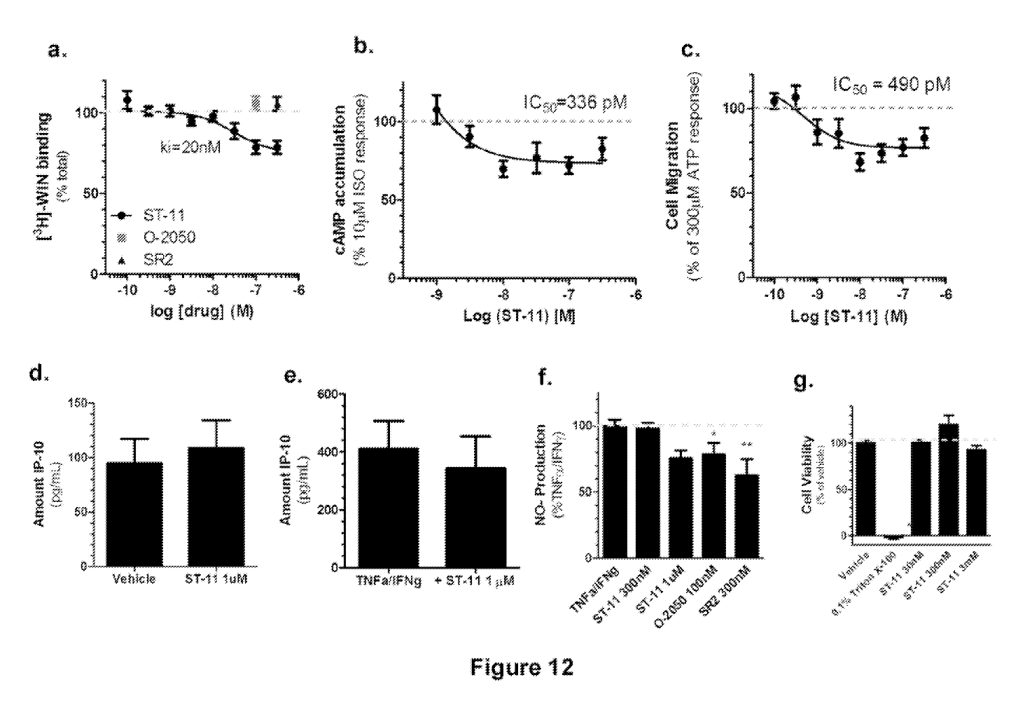

Evidence indicates that immune cells, including microglial cells, express AI receptors. FIG. 12 shows that mouse microglia in primary culture express AI receptors as suggested by [³H]-WIN55212 binding competed by ST-11 (a). ST-11 inhibits cAMP production stimulated by Isoproterenol (b) and microglia cell migration stimulated by ATP (c). ST-11 does not stimulate or inhibit IP production (d & e), NO production (f) and affect cell viability in microglia. Together these results suggest that AI receptors are expressed by both mouse astrocytomas and microglial cells, indicating that ST-compounds influence brain tumor pathogenesis.

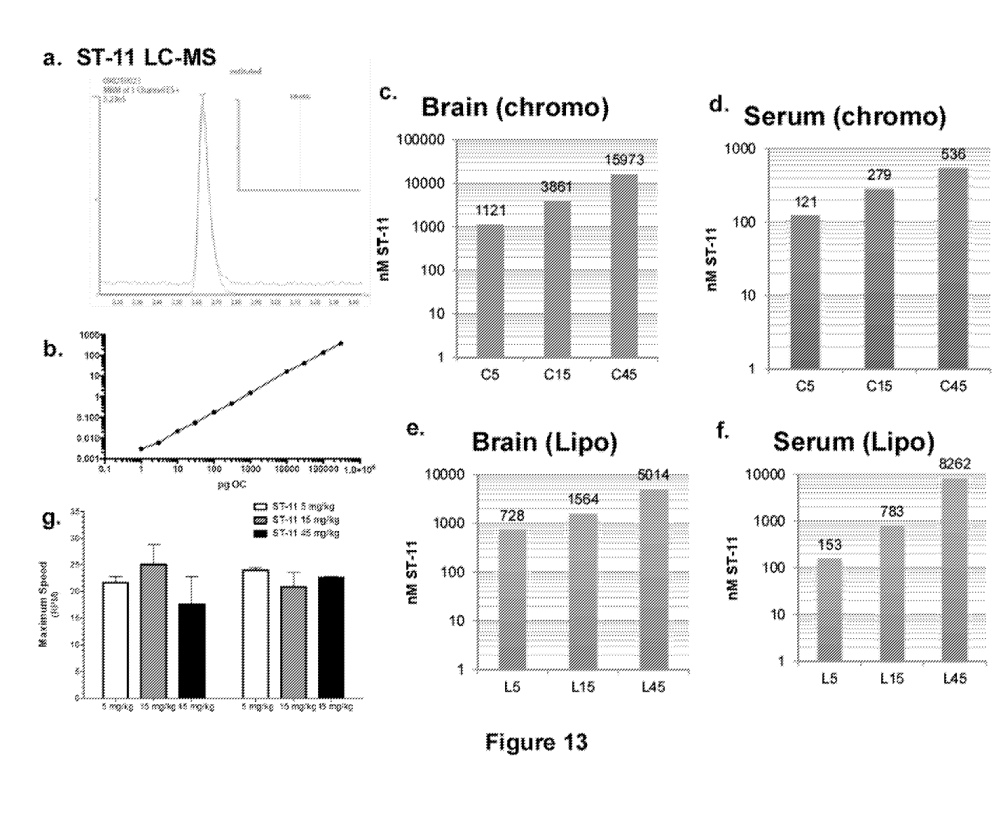

As an initial step to test the therapeutic efficacy of ST-11 in vivo, the pharmacokinetic profile and acme toxicity profile of ST-11 in healthy mice were determined. FIG. 13 illustrates an LC-MS chromatogram and calibration curve of ST-11 (a & 13). PK profile of ST-11 (c, d, e & f). ST-11 does not influence locomotor activity on an accelerating rotarod, suggesting lack of acute toxicity.

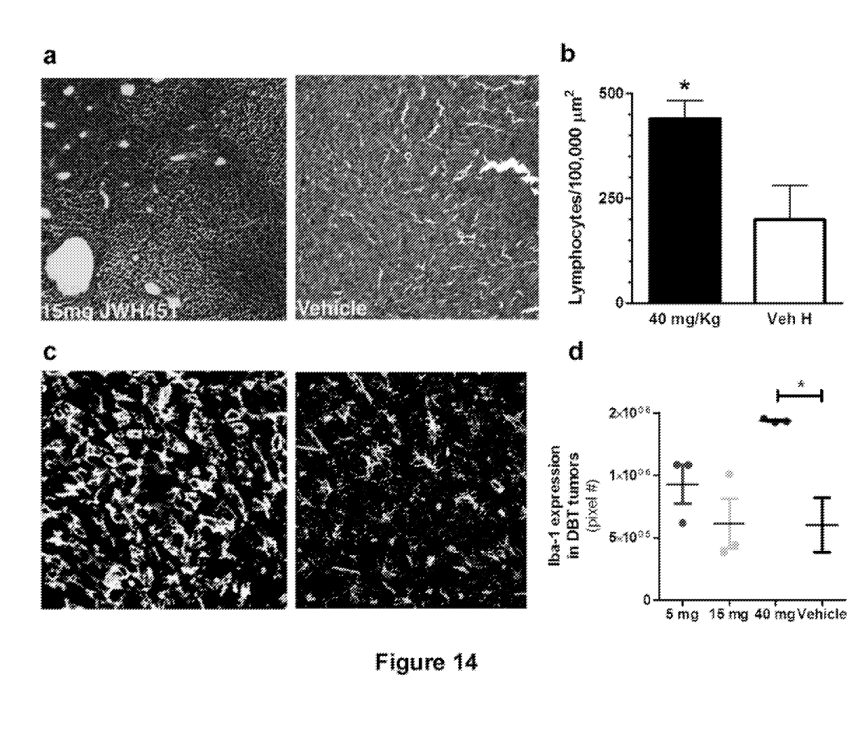

FIG. 14 shows that ST-11 increases the number of lymphocytes (a, b) and microglia (c, d) in DBT tumors implanted in BalbC mice and treated daily ova 2 weeks.

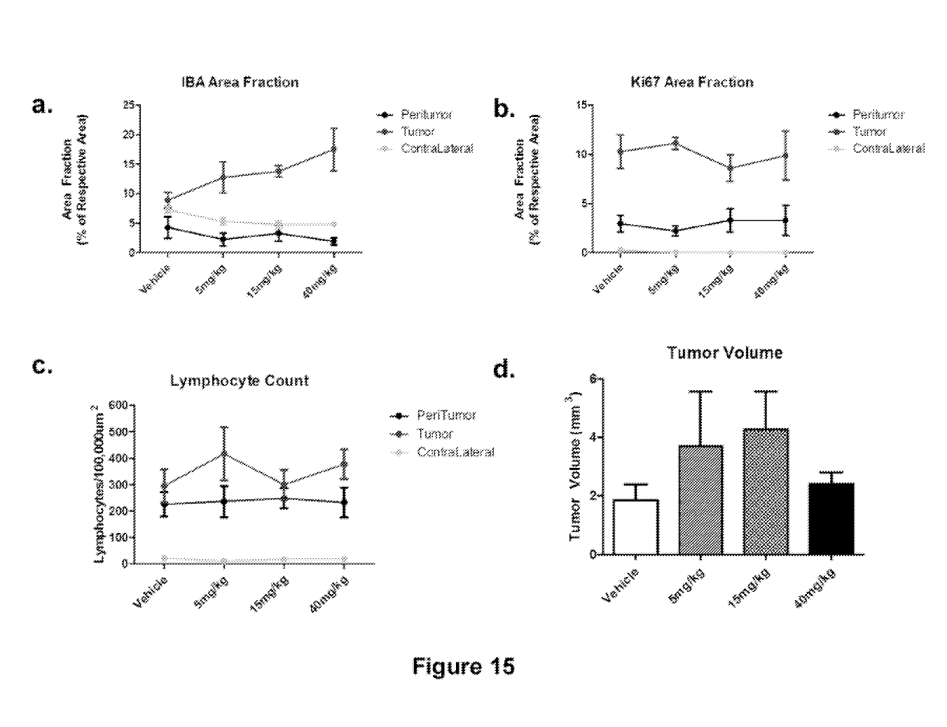

FIG. 15 shows the effect of ST-11 on mouse microglia (a) and lymphocyte cell number (c), as well as on cell division (b) and overall tumor volume (d) i in DBT tumors implanted in BalbC mice and treated daily over 2 weeks.

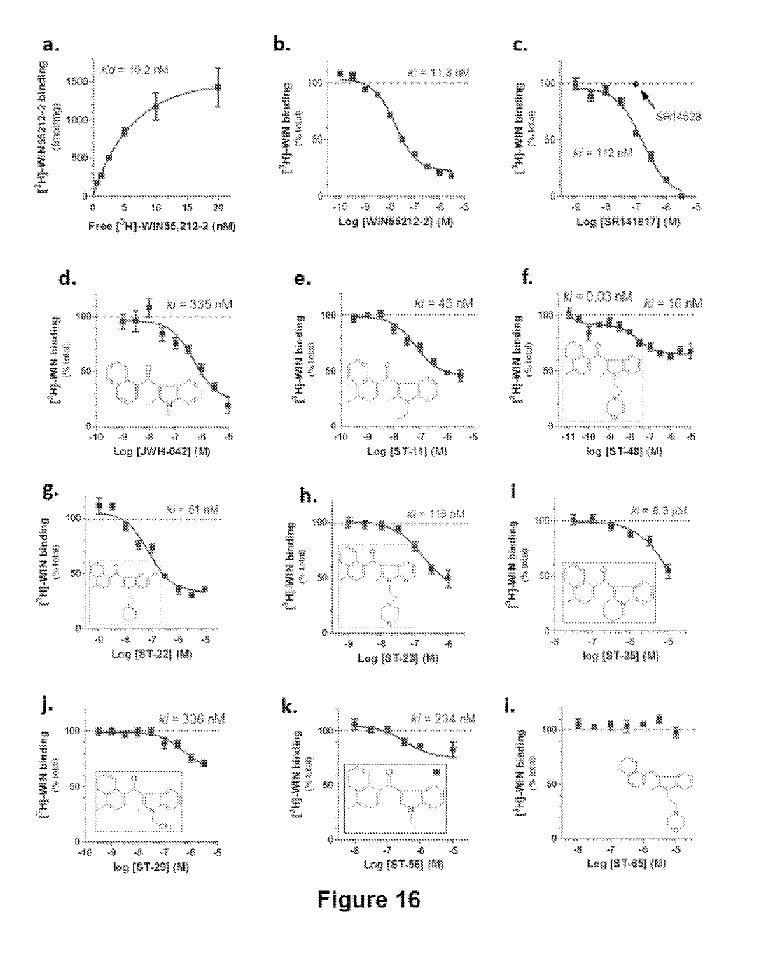

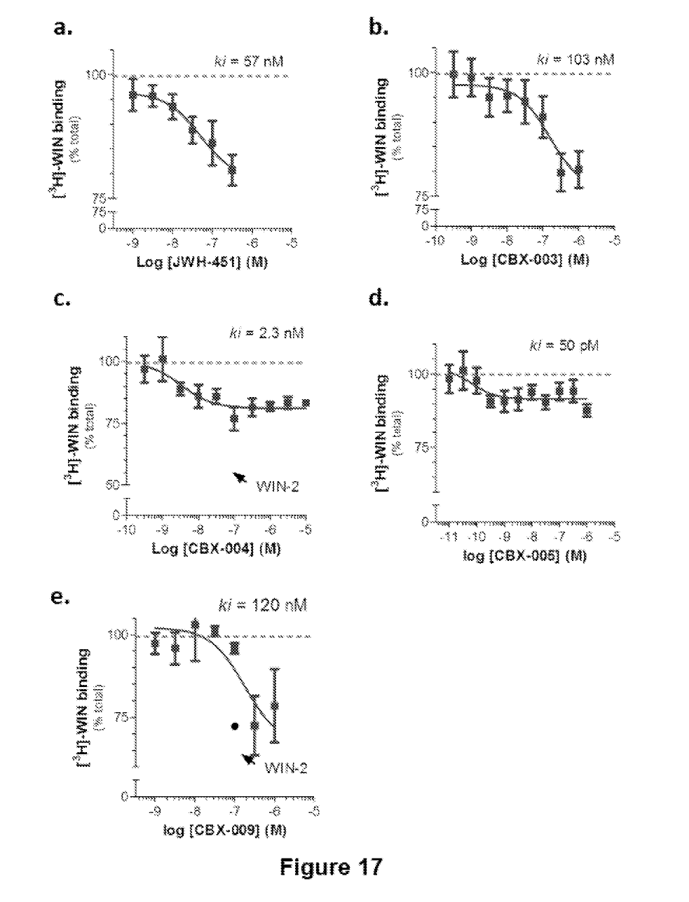

AI receptors were evaluated Ear their expression by neurons. FIG. 16 shows that ST-compounds compete for [³H]-WIN55212-2 binding in sknmc cells, a human neuronal cell line, suggesting that these cells express AI receptors. FIG. 17 shows that ST-compounds compete for [³H]-WIN55212-2 binding in CB₁−/− mouse brain homogenates, suggesting that mouse neurons express AI receptors. Together these results suggest that neuron express significant level of AI receptors both in culture and in mice brain.

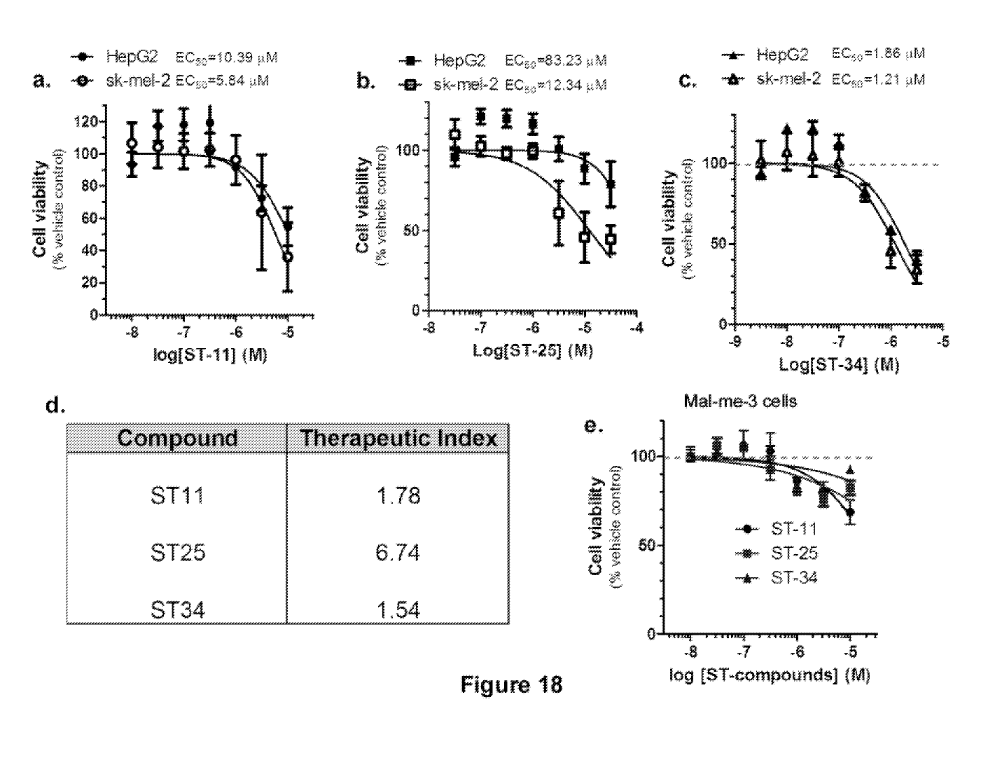

Astrocytoma and melanoma develop from common precursors and lineages, suggesting that both types of cancers might respond similarly to therapeutics. FIG. 18 illustrates the potency of ST compounds at killing human melanoma cells lines in culture

### Example 9

### 9-ethyl-9H-carbazol-3-yl)(piperidin-1-yl)methanone, ST-53

### Synthesis of 9-ethyl-carbazole (compound 9.1

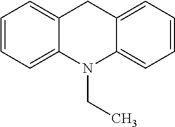 [SMILES: CCN1c2ccccc2Cc2ccccc21]

Under argon atmosphere, a solution of carbazole (5.0 g, 29.93 mmol), 1-bromopethane (5.9 mL, 37.41 mmol), and Cs₂CO₃ (19.5 g, 59.86 mmol) in DMF (10 mL) was subjected to microwave irradiation at 140° C. for 2 h. The reaction mixture was cooled, diluted with ethyl acetate (50 mL), and filtered. The organic solvents were evaporated in vacuo. The resultant dark oil was purified by column chromatography on silica gel using heptanes/ethyl acetate in different proportions to afford the title compound as yellowish oil (5.409 g, 92.6%).

### Synthesis of 9-ethyl-9H-carbazole-3-carbaldehyde (compound 9.2)

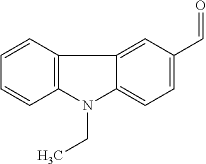 [SMILES: CCn1c2ccccc2c2cc(C=O)ccc21]

POCl₃ (2.3 mL, 10.25 mmol) was added, over a period of 10 min, to an ice-cooled, stirred DMF (5 mL) and Carbazole 9.1 (1.0 g, 5.13 mmol) under argon. The solution was allowed to stir at room temperature for 16 h. The reaction mixture was cooled and then poured into crushed ice. After warming to room temperature, the resultant product was extracted into ethyl acetate, and the organic phase was washed with water, brine, dried (MgSO₄), filtered, and evaporated in vacuo. The obtained residue was purified by column chromatography on silica gel using heptanes/ethyl acetate in different proportions to afford the title compound as a white solid (1.016 g, 88.8%).

### Synthesis of 9-ethyl-9H-carbazole-3-carboxylic acid (compound 9.3)

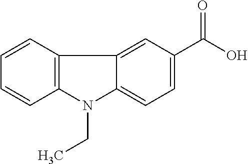 [SMILES: CCn1c2ccccc2c2cc(C(=O)O)ccc21]

To a cold solution (ice bath) of 9-ethyl-3-formylcarbazole 9.2 (1.0 g, 4.48 mmol) in water/acetone (50 mL, 1:1. v/v) was added dropwise with stirring a solution of potassium permanganate (1.4 g, 8.96 mmol) in acetone (50 mL). The mixture was heated 5 h at reflux and allowed to cool to room temperature. The mixture was filtered through a pad of celite and concentrated in vacuo to remove acetone. The obtained solution was diluted with water (100 mL), basified with NaOH to pH ca. 10, and extracted with heptane/ether (4:1, v/v, 50 mL×3) to remove the unreacted starting material. The aqueous solution was cooled on an ice-water bath and acidified with ice-cold solution of sulfuric acid (20%) to pH ca. 2. The resultant bulky precipitate was extracted into ethyl acetate and the extract was washed with brine, dried over magnesium sulfate, filtered, and concentrated in vacuo. The precipitated product was collected by filtration, washed several times with cyclohexanes, and dried overnight to produce the title compound 3 (973 mg, 90.8%) as a greenish solid.

### Synthesis of (9-ethyl-carbazol-3-yl)(1,2,3,4-tetrahydraquinoline)methanone (compound 9.4) ST-53

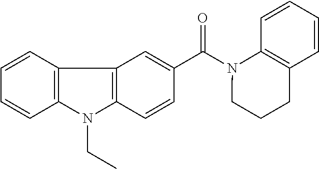 [SMILES: CCn1c2ccccc2c2cc(C(=O)N3CCCc4ccccc43)ccc21]

9H-carbazole-3-carboxylic acid 9.3 (300 mg, 1.07 mmol), 1,2,3,4-tetrahydraquinoline (215 mg, 2.53 mmol), DIPEA (363 mL, 2.14 mmol), and DMAP (156 mg, 1.28 mmol) were added to DCM (30 mL) under argon. EDC (350 mg, 1.83 mmol) was added to the solution, and the reaction mixture was then stirred for 16 h. The solvent was removed in vacuo, and the obtained residue was extracted into ethyl acetate (100 mL), The organic layer was washed consecutively with 5% citric acid solution (50 mL×3), concentrated sodium bicarbonate (50 mL×3), brine (50 mL), dried over magnesium sulfate, filtered, and concentrated in vacuo. The residue was purified on silica gel using heptanes/ethyl acetate in different proportions to afford the title compound as a yellowish glass (345 mg, 93%). LC/MS m/s [M+H]⁺ 355.1831

### Example 10

### (9-ethyl-carbazol-3-yl)(N-methyl piperazine)methanone, ST-62

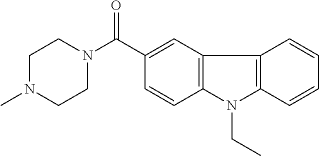 [SMILES: CCn1c2ccccc2c2cc(C(=O)N3CCN(C)CC3)ccc21]

Using 9H-carbazole-3-carboxylic acid 9.3 (300 mg, 0.40 mmol) and N-methyl piperazine (74 mL, 0.71 mmol) as starting compounds, the title compound was prepared following the procedures described in preparation of compound 4. A colorless viscous oil was obtained. Yield: 44 mg (33%). LC/MS m/s [M+H]⁺ 322.1952.

### Example 11

### (9-ethyl-carbazol-3-yl)(p-methylphenyl)methanone, ST-63

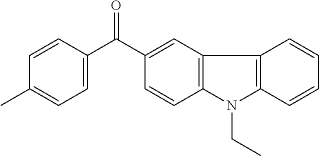 [SMILES: CCn1c2ccccc2c2cc(C(=O)c3ccc(C)cc3)ccc21]

Under argon atmosphere, AlCl₃ (309 mg, 2.32 mmol) was added to a solution of carbazole 9.1 (200 mg, 2.11 mmol) in dry benzene (30) mL, and the obtained solution was placed in an ice-water bath for 20 min. p-Methylbenzoyl chloride (282 mL, 2.43 mmol) was added dropwise via a syringe to the solution, and the reaction mixture was then allowed to warm to room temperature and stirred for 16 h. The reaction mixture was cooled on an ice-water bath then poured onto a mixture of ice and concentrated NaOH and extracted with ethyl acetate. The organic phase was washed with saturated aqueous sodium bicarbonate, brine, dried (MgSO₄), filtered and evaporated in vacuo. The obtained residue was purified by column chromatography on silica gel eliding with ethyl acetate/heptanes in different proportions to give ST-63 (514 mg, 71%) as a greenish oil. LC/MS m/s [M+H]⁺ 314.1545.

### Example 12

### (9-ethyl-carbazol-3-yl)(8-quinoline)methanone

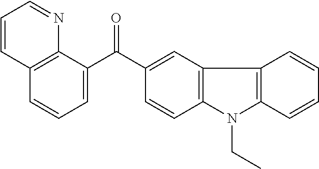 [SMILES: CCn1c2ccccc2c2cc(C(=O)c3cccc4cccnc34)ccc21]

Using 9-ethyl-carbazole-3-carboxylic acid 9.1 (190 mg, 0.97 mmol) and 8-quinoline acid chloride (279 mg, 1.46 mmol) as starting compounds, the title compound was prepared following the procedures described in preparation of example 11. A yellowish viscous oil was obtained. Yield: 124.6 mg (36.5%). LC/MS m/s [M+H]⁺351.1500.

### Example 13

### (9-ethyl-carbazol-3-yl)(5-quinoline)methanone

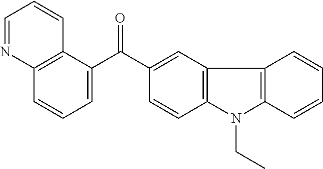 [SMILES: CCn1c2ccccc2c2cc(C(=O)c3cccc4ncccc34)ccc21]

Using 9-ethyl-carbazole-3-carboxylic acid 9.1 (190 mg, 0.97 mmol) and 5-quinoline acid chloride (279 mg, 1.46 mmol) as starting compounds, the title compound was prepared following the procedures described in preparation of example 11. A yellowish viscous oil was obtained. Yield: 119.2 mg (35%). LC/MS m/s [M+H]⁺ 351.1487

### Example 14

### (9-ethyl-carbazol-3-yl)(naphthyl)methanone

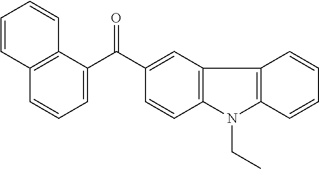 [SMILES: CCn1c2ccccc2c2cc(C(=O)c3cccc4ccccc34)ccc21]

Using 9-ethyl-carbazole-3-carboxylic acid 9.1 (100 mg, 0.40 mmol) and 1-naphthoyl chloride (74 mL, 0.71 mmol) as starting compounds, the title compound was prepared following the procedures described in preparation of example 11. A yellowish viscous oil was obtained. Yield: 141.1 mg (41.5%). LC/MS m/s [M+H]⁺ 350.1536.

### Example 15

### (9-ethyl-carbazol-3-yl)(phenylacetyl)methanone

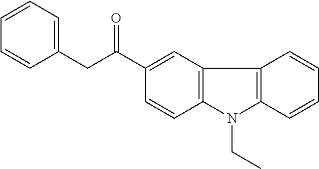 [SMILES: CCn1c2ccccc2c2cc(C(=O)Cc3ccccc3)ccc21]

Using 9-ethyl-carbazole-3-carboxylic acid 9.1 (200 mg, 1.03 mmol) and phenylacetyl chloride (154.02 mL, 1.54 mmol) as starting compounds, the title compound was prepared following the procedures described in preparation of example 11. A yellowish viscous oil was obtained. Yield: 127.3 mg (633%). LC/MS m/s [M+H]⁺314.1530.

### Example 16

### (9-ethyl-carbazol-3-yl)(5,6,7,8-tetrahydra-8-quinoline)methanone

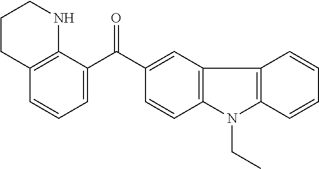 [SMILES: CCn1c2ccccc2c2cc(C(=O)c3cccc4c3NCCC4)ccc21]

To a solution of example 12 (40 mg) and THF was added sodium cyanobocohydride and borohydride THF complex in THF (10 ml) under argon. The solution stirred for 24 hours. Every eight hours another equivalent of sodium cyanoborohydride and borohydride THF complex was added. Upon completion of the reaction, the product was then extracted with EtOAc (3×10 mL) from water (50 mL), and dried over MgSO₄. The obtained residue was purified by column chromatography on silica gel eluting with ethyl acetate/heptanes in different proportions to obtain the pure product. Yield: 10 mg (33%). LC/MS m/s [M+H]⁺ 355.1814.

### Example 17

### (9-ethyl-carbazol-3-yl)(5,6,7,8-tetrahydra-5-quinoline)methanone

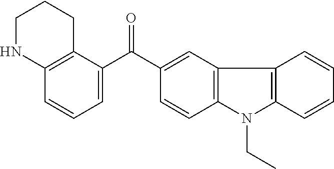 [SMILES: CCn1c2ccccc2c2cc(C(=O)c3cccc4c3CCCN4)ccc21]

To a solution of example 13 (40 mg) and THF was added sodium cyanoborohydride and borohydride THF complex in THF (10 ml) under argon. The solution stirred for 24 hours. Every eight hours another equivalent of sodium cyanoborohydride and borohydride THF complex was added. Upon completion of the reaction, the product was then extracted with EtOAc (3×10 mL) from water (50 mL), and dried over MgSO₄. The obtained residue was purified by column chromatography on silica gel eluting with ethyl acetate/heptanes in different proportions to obtain the pure product. LC/MS m/s [M+H]⁺ 355.1814.

### Example 18

### 9-propyl-carbazol-3-yl)(4-methyl-naphthyl)methanone ST-58

### Synthesis of 9-propylcarbazole (compound 18.1

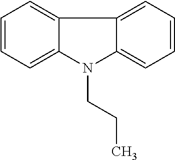 [SMILES: CCCn1c2ccccc2c2ccccc21]

Using carbazole (500 mg, 2.70 mmol) and 1-iodopropane (0.84 mL, 3.38 mmol) as starting compounds, the title compound was prepared following the procedures described in preparation of compound 9.1. A viscous oil was obtained. Yield: 469.4 mg (83.1%).

### Synthesis of (9-propyl-carbazol-3-yl)(4-methyl-naphthyl)methanone (compound 18.2)

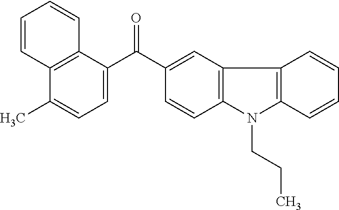 [SMILES: CCCn1c2ccccc2c2cc(C(=O)c3ccc(C)c4ccccc34)ccc21]

Using 9-ethyl-carbazole-3-carboxylic acid 18.1 (350 mg, 1.67 mmol) and 4-methyl 1-naphthoyl chloride (245.6 mg, 2.0 mmol) as starting compounds, the title compound was prepared following the procedures described in preparation of example 11. A yellowish viscous oil was of ST-58 was obtained. Yield: 191.4 mg (30.3%). LC/MS m/s [M+H]⁺ 378.1851.

### Example 19

### 2-(4-methylnaphthoyl)-5-ethyl-1H,2H,3H,4H,5H-pyrido[4,3-b]indole, ST-60

### Synthesis of ethyl 3,4-dihydro-1H-pyrido[4,3-b]indole-2(5H)-carboxylate (compound 19.1)

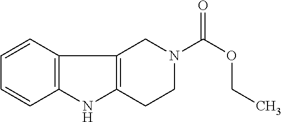 [SMILES: CCOC(=O)N1CCc2[nH]c3ccccc3c2C1]

The Carboline 19.1 was prepared by heating phenylhydrazine (3.64 mL, 36.9 mmol) and 1-carbethoxy-4-piperidone (6.7 mL, 44.4 mmol) in anhydrous ethanol (50 mL) at reflux for 16 h. The solvent was evaporated in vacuo, and the obtained residue was purified by silica gel chromatography using ethyl acetate/heptanes in different proportions to afford the title compound as a white solid. Yield 5.466 g (60.5%).

### Synthesis of ethyl 5-ethyl-3,4-dihydro-1H-pyrido[4,3-b]indole-2(5H)-carboxylate (compound 19.2)

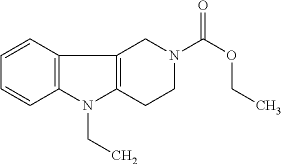 [SMILES: CCOC(=O)N1CCc2c(c3ccccc3n2CC)C1]

Under argon atmosphere, a solution of carboline 19.1 (2.75 g, 11.26 mmol), ethyl bromide (1.26 mL, 16.89 mmol) in DMF (10 mL) was cooled to 0° C., After 15 minutes NaH (900 mg, 22.53 mmol) was added to the solution. The mixture was then allowed to warm to rt and stirred for 2 h. The reaction mixture was diluted with ethyl acetate and filtered. The organic solvents were evaporated in vacuo. The residue was suspended in ethyl acetate (150 mL), and the organic phase was washed with saturated aqueous sodium bicarbonate, brine, dried (MgSO₄), filtered and evaporated in vacuo. The obtained residue was purified by column chromatography on silica gel using ethyl acetate/heptanes in different proportions to afford the title compound (2.316 mg, 75.3%) as a white solid.

### Synthesis of 2(4-methylnaphthoyl)-5-ethyl-1H,2H,3H,4H,5H-pyrido[4,3-b]indole (compound 19.3)

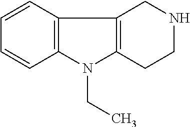

Solid KOH (3 g, 53.57 mmol) was added to a solution of carbethoxyindole 19.2 (2.0 g, 7.35 mmol) in a mixture methanol (80 mL) and water (10 mL). The resulting solution was heated at reflux for 24 h. The obtained solution was concentrated in vacuo to remove ethanol, diluted with saturated aqueous sodium bicarbonate (50 mL) and extracted with ethyl acetate (150 mL). The organic phase was washed with saturated aqueous sodium bicarbonate (50 mL×2), brine (50 mL), dried (MgSO₄), filtered and evaporated in vacuo. The obtained residue was purified by column chromatography on silica gel using ethyl acetate/heptanes in different proportions to afford the title compound 19.3: 973 mg (66.1%),

### Synthesis of 2-(4-methylnaphthoyl)-5-ethyl-1H,2H,3H,4H,5H-pyrido[4,3-b]indole, ST-60

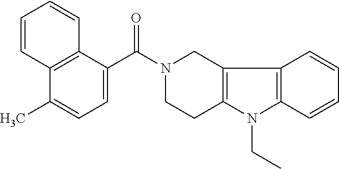 [SMILES: CCn1c2c(c3ccccc31)CN(C(=O)c1ccc(C)c3ccccc13)CC2]

Using amine 19.3 (250 mg, 1.25 mmol) and 4-methyl-1-napthylic acid (348.2 mg, 1.87 mmol) as starting compounds, the title compound was prepared following the procedures described in preparation of example 2 as a yellowish oil. Yield: 215.6 mg (41.4%). LC/MS m/s [M+H]⁺ 369.1936.

### Example 20

### 8-(4-methyl-naphthyl)-5-ethyl-1H,2H,3H,4H,5H-pyrido[4,3-b]indole-2-carboxylate ST-54

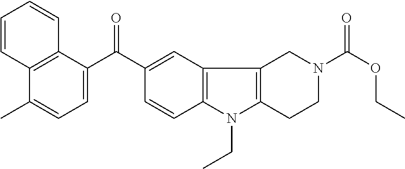 [SMILES: CCOC(=O)N1CCc2c(c3cc(C(=O)c4ccc(C)c5ccccc45)ccc3n2CC)C1]

Using amine 19.1 (100 mg, 0.32 mmol) and 4-methylnaphthalene-1-carbonyl chloride (131 mg, 0.49 mmol) as starting compounds, the title compound was prepared following the procedures described in preparation of compound ST-54 as a dark viscous oil. Yield: 88%. LC/MS m/s [M+H]⁺441.3099.

### Example 21

### 2-(7-Fluorobenzofurazan-4-sulfonoyl)-5-ethyl-1H,2H,3H,4H,5H-pyrido[4,3-b]indole ST-59

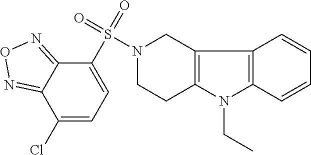 [SMILES: CCn1c2c(c3ccccc31)CN(S(=O)(=O)c1ccc(Cl)c3nonc13)CC2]

Amine 19.3 (250 mg, 1.25 mmol) was suspended in anhydrous DCM (30 mL) under argon, and the obtained suspension was cooled with ice-cold water. Triethyl amine (1.0 mL) was added to the solution, followed by 7-chlorobenzofurazan-4-sulfonic acid chloride (377.7 mg, 1.49 mmol). The flask was removed from the ice bath, and the reaction mixture was stirred at rt for 4 h. After concentration, the residue was purified by column chromatography on silica gel, eluting with EtOAc/heptanes in different proportions to afford 324.2 mg (62.3%) of ST-59 as an orange oil. LC/MS m/s [M+H]⁺417.076.

### Example 22

### 9-(4-methyl-naphthyl)-carbazol-3-yl, ST-64

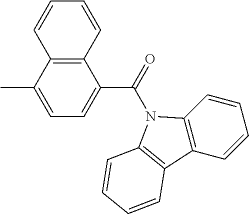 [SMILES: Cc1ccc(C(=O)n2c3ccccc3c3ccccc32)c2ccccc12]

Using carbazole (250 mg, 1.35 mmol) and 4-methyl-1-napthylic acid (344.5 mg, 1.69 mmol) as starting compounds, the title compound was prepared following the procedures described in preparation of ST-59 as a dark oil. Yield: 341.6 mg (75.5%). LC/MS m/s [M+H]⁺ 336.14.

The disclosures of all articles and references mentioned in this application, including patents, are incorporated herein by reference in their entirety.

It is understood that the examples and embodiments described herein are for illustrative purposes only. Unless dearly excluded by the context, all embodiments disclosed for one aspect of the invention can be combined with embodiments disclosed for other aspects of the invention, in any suitable combination. It will be apparent to those skilled in the art that various modifications and variations can be made to the present invention without departing from the scope of the invention. Thus, it is intended that the present invention cover the modifications and variations of this invention provided they come within the scope of the appended claims and their equivalents. All publications, patents, and patent applications cited herein are hereby incorporated herein by reference for all purposes.

1. A compound which is:

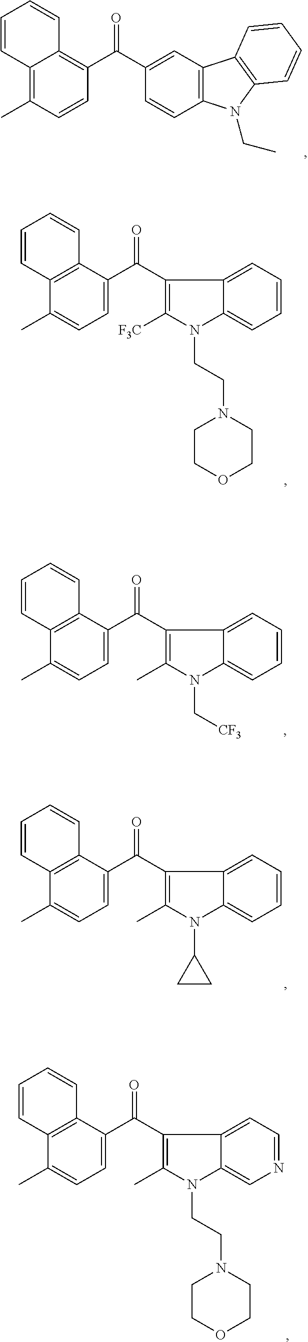 [SMILES: CCn1c2ccccc2c2cc(C(=O)c3ccc(C)c4ccccc34)ccc21 ; Cc1ccc(C(=O)c2c(C(F)(F)F)n(CCN3CCOCC3)c3ccccc23)c2ccccc12 ; Cc1ccc(C(=O)c2c(C)n(CC(F)(F)F)c3ccccc23)c2ccccc12 ; Cc1ccc(C(=O)c2c(C)n(C3CC3)c3ccccc23)c2ccccc12 ; Cc1ccc(C(=O)c2c(C)n(CCN3CCOCC3)c3cnccc23)c2ccccc12]

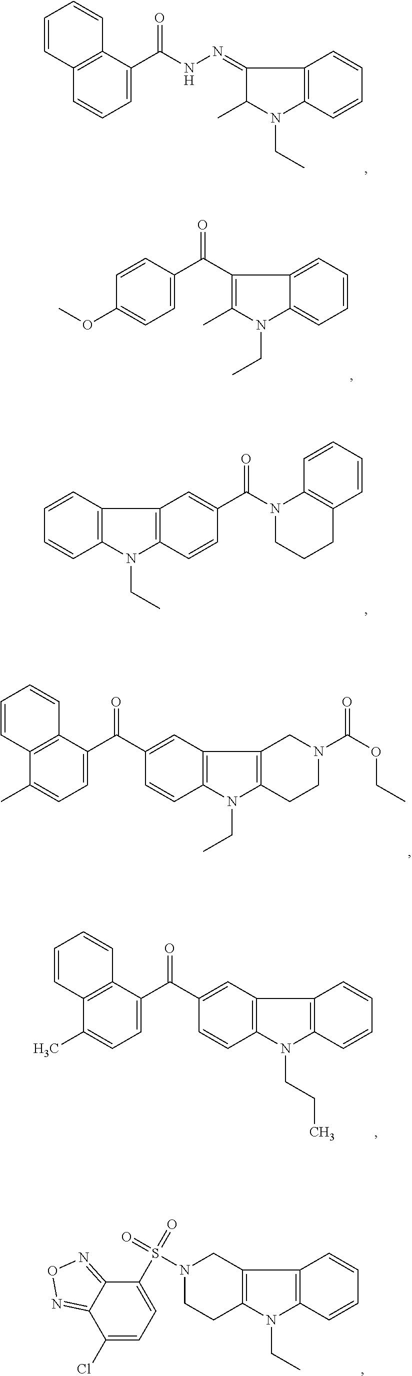 [SMILES: CCN1c2ccccc2/C(=N/NC(=O)c2cccc3ccccc23)C1C ; CCn1c(C)c(C(=O)c2ccc(OC)cc2)c2ccccc21 ; CCn1c2ccccc2c2cc(C(=O)N3CCCc4ccccc43)ccc21 ; CCOC(=O)N1CCc2c(c3cc(C(=O)c4ccc(C)c5ccccc45)ccc3n2CC)C1 ; CCCn1c2ccccc2c2cc(C(=O)c3ccc(C)c4ccccc34)ccc21 ; CCn1c2c(c3ccccc31)CN(S(=O)(=O)c1ccc(Cl)c3nonc13)CC2]

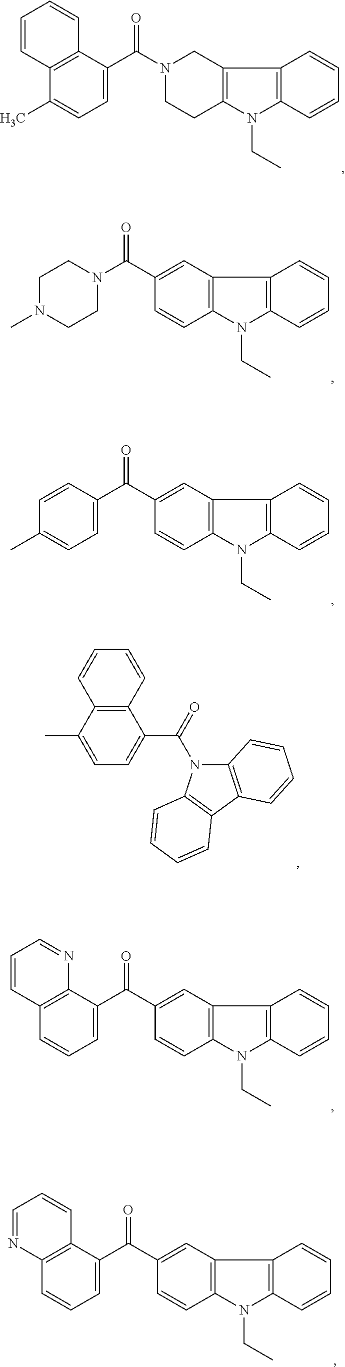 [SMILES: CCn1c2c(c3ccccc31)CN(C(=O)c1ccc(C)c3ccccc13)CC2 ; CCn1c2ccccc2c2cc(C(=O)N3CCN(C)CC3)ccc21 ; CCn1c2ccccc2c2cc(C(=O)c3ccc(C)cc3)ccc21 ; Cc1ccc(C(=O)n2c3ccccc3c3ccccc32)c2ccccc12 ; CCn1c2ccccc2c2cc(C(=O)c3cccc4cccnc34)ccc21 ; CCn1c2ccccc2c2cc(C(=O)c3cccc4ncccc34)ccc21]

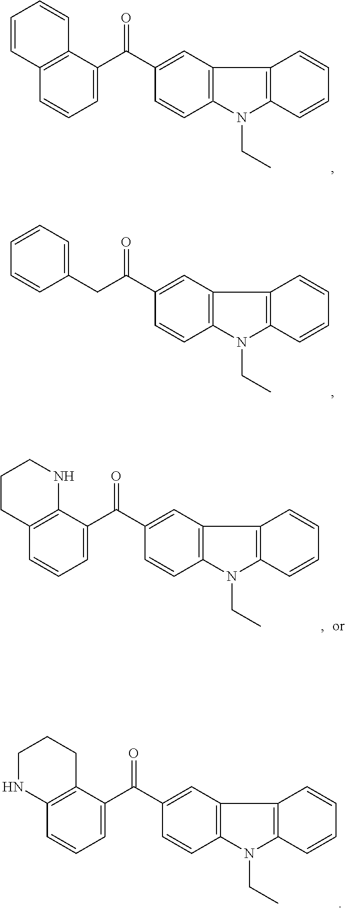 [SMILES: CCn1c2ccccc2c2cc(C(=O)c3cccc4ccccc34)ccc21 ; CCn1c2ccccc2c2cc(C(=O)Cc3ccccc3)ccc21 ; CCn1c2ccccc2c2cc(C(=O)c3cccc4c3NCCC4)ccc21 ; CCn1c2ccccc2c2cc(C(=O)c3cccc4c3CCCN4)ccc21]

2. A pharmaceutical composition comprising a compound of claim 1 and one or more pharmaceutically acceptable diluent, preservative, solubilizer, emulsifier, adjuvant, excipient, or carrier.

3. A method of treating cancer, acognition disorder, schizophrenia, Alzheimer's disease and dementia, Parkinson's disease, depression, multiple sclerosis, amyotrophic lateral sclerosis (ALS), Huntington's disease, Fronto temporal dementia, parkinsonism linked to chromosome 17, or prion diseases in a subject, comprising administering to the subject an effective amount of a compound of claim 1.

4. The method of claim 3, wherein the cancer is selected from the group consisting of glioblastoma, melanoma, breast cancer, medulloblastomas, astrocytoma, and colon cancer.

5. The method according to claim 4, wherein the cancer is glioblastoma or astrocytoma.

6. The method according to claim 4, wherein the cancer is melanoma.

7. (canceled)

8. The method of claim 7, wherein the compound is:

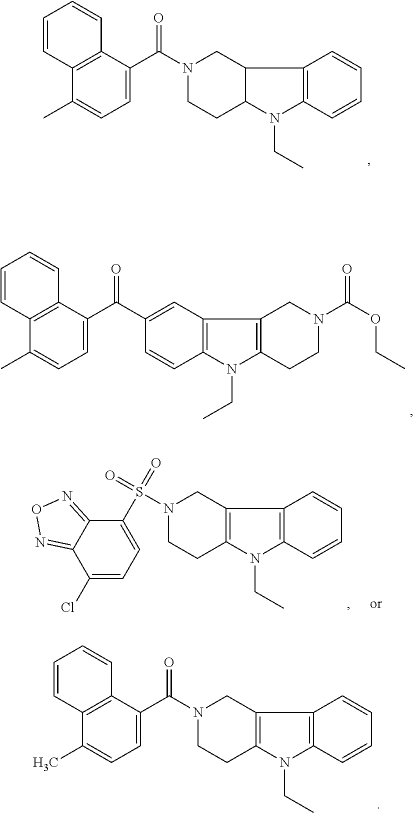

9. The method of claim 7, wherein the compound is:

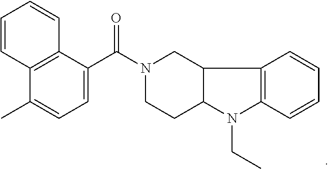 [SMILES: CCN1c2ccccc2C2CN(C(=O)c3ccc(C)c4ccccc34)CCC21]

10. The method of claim 7, wherein the cancer is selected from the group consisting of glioblastoma, melanoma, breast cancer, medulloblastomas, astrocytoma, and colon cancer.

11. The method according to claim 10, wherein the cancer is glioblastoma or astrocytoma.

12. The method according to claim 10, wherein the cancer is melanoma.

13. A method of treating cancer in a subject, comprising administering to the subject an effective amount of the compound of claim 19, wherein the compound is selected from the group consisting of:

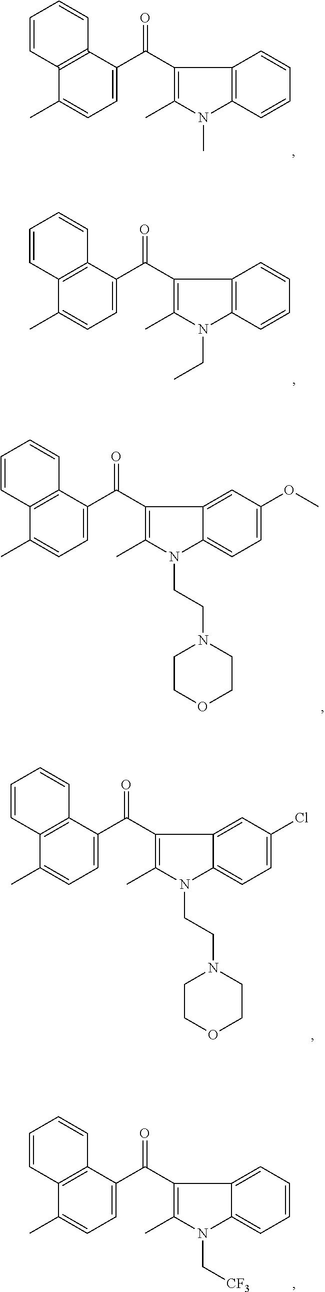 [SMILES: Cc1ccc(C(=O)c2c(C)n(C)c3ccccc23)c2ccccc12 ; CCn1c(C)c(C(=O)c2ccc(C)c3ccccc23)c2ccccc21 ; COc1ccc2c(c1)c(C(=O)c1ccc(C)c3ccccc13)c(C)n2CCN1CCOCC1 ; Cc1ccc(C(=O)c2c(C)n(CCN3CCOCC3)c3ccc(Cl)cc23)c2ccccc12 ; Cc1ccc(C(=O)c2c(C)n(CC(F)(F)F)c3ccccc23)c2ccccc12]

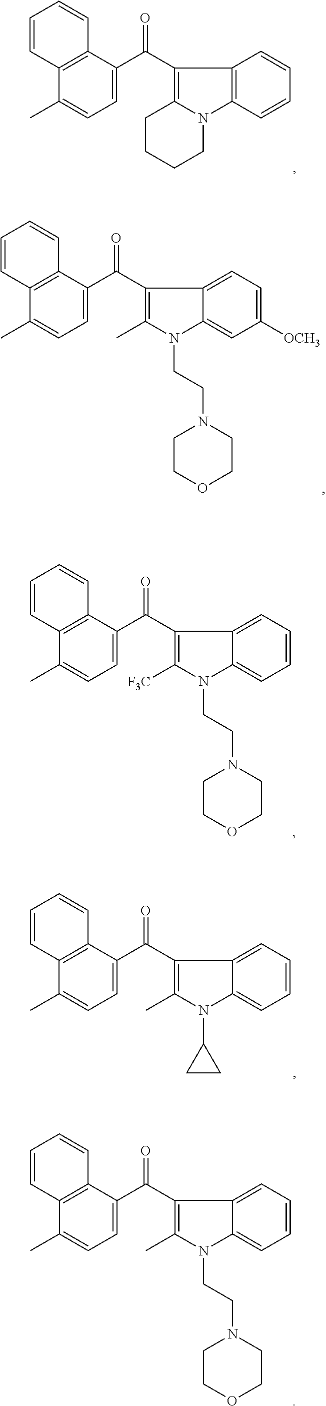 [SMILES: Cc1ccc(C(=O)c2c3n(c4ccccc24)CCCC3)c2ccccc12 ; COc1ccc2c(C(=O)c3ccc(C)c4ccccc34)c(C)n(CCN3CCOCC3)c2c1 ; Cc1ccc(C(=O)c2c(C(F)(F)F)n(CCN3CCOCC3)c3ccccc23)c2ccccc12 ; Cc1ccc(C(=O)c2c(C)n(C3CC3)c3ccccc23)c2ccccc12 ; Cc1ccc(C(=O)c2c(C)n(CCN3CCOCC3)c3ccccc23)c2ccccc12]

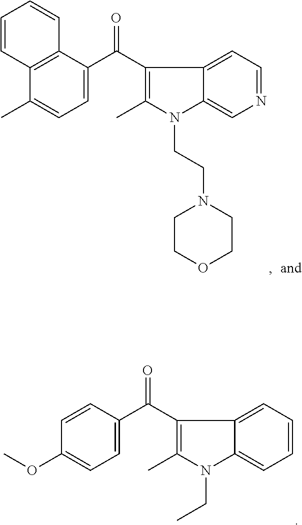 [SMILES: Cc1ccc(C(=O)c2c(C)n(CCN3CCOCC3)c3cnccc23)c2ccccc12 ; CCn1c(C)c(C(=O)c2ccc(OC)cc2)c2ccccc21]

14. The method according to claim 13, wherein the compound is:

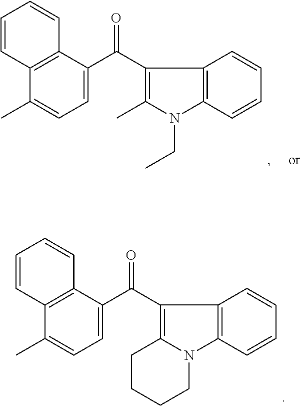 [SMILES: CCn1c(C)c(C(=O)c2ccc(C)c3ccccc23)c2ccccc21 ; Cc1ccc(C(=O)c2c3n(c4ccccc24)CCCC3)c2ccccc12]

15. The method of claim 13, wherein the cancer is glioblastoma, melanoma, breast cancer, medulloblastomas, astrocytoma, or colon cancer.

16. The method according to claim 15, the cancer is glioblastoma or astrocytoma.

17. (canceled)

18. A compound of formula (I) 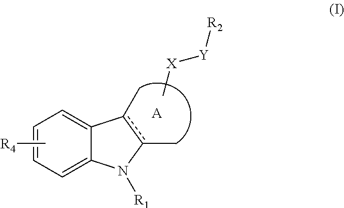
or a salt of prodrug of, wherein:
ring A is a saturated or unsaturated 6 or 7-member ring, which can optionally contain one or more nitrogen atoms, and is optionally substituted with R₉;
X is selected from the group consisting of CH(OH), C═O, C═S, and S(O)₁₋₂;
Y is selected from the group consisting of absent, O, N(R₃), and C(R₃)(R₈);
R₁ is selected from the group consisting of optionally substituted alkyl, optionally substituted alkynyl, optionally substituted alkynyl, optionally substituted alkylcarbonyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₂ is selected from the group consisting of optionally substituted alkyl, optionally substituted alkynyl, optionally substituted alkynyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₃ is selected from the group consisting of optionally substituted alkyl, optionally substituted alkynyl, optionally substituted alkynyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical; or
or R₂ and R₃, with the atoms to which they are attached form an optionally substituted cycloalkyl, an optionally substituted heteroaryl, an optionally substituted heterocyclyl, or an optionally substituted aryl;
R₄ is selected from the group consisting of hydrogen, optionally substituted alkyl, optionally substituted alkynyl, optionally substituted alkynyl, optionally substituted cycloalkyl, optionally substituted aryl, optionally substituted aralkyl, optionally substituted heteroaryl, optionally substituted heterocyclyl, halogen, optionally substituted alkoxy, optionally substituted haloalkoxy, hydroxyl, N(R₅)(R₆), and polyether radical;
R₅ and R₆ are independently selected from the group consisting of hydrogen, optionally substituted alkyl, optionally substituted acyl, optionally substituted heteroalkyl, optionally substituted aryl, optionally substituted cycloalkyl, optionally substituted heteroaryl, and optionally substituted heterocyclyl;
R₈ is selected from the group consisting of hydrogen, optionally substituted alkyl, optionally substituted alkenyl, optionally substituted alkynyl, optionally substituted cycloalkyl, optionally substituted aryl, optionally substituted aralkyl, a halogen, optionally substituted alkoxy, and hydroxyl, or can form an optionally substituted cycloalkyl, an optionally substituted heteroaryl, an optionally substituted heterocyclyl, or an optionally substituted aryl with R₃; and R₉ is selected from the group consisting of hydrogen, optionally substituted alkyl, optionally substituted alkynyl, optionally substituted alkynyl, optionally substituted cycloalkyl, optionally substituted aryl, optionally substituted aralkyl, optionally substituted heteroaryl, optionally substituted heterocyclyl, halogen, optionally substituted alkoxy, optionally substituted haloalkoxy, hydroxyl, and N(R₅)(R₆).

19. A compound of formula (II) 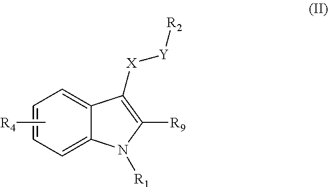
or a salt of prodrug of, wherein:
X is selected from the group consisting of CH(OH), C═O, C═S, and S(O)₁₋₂;
Y is selected from the group consisting of absent, O, N(R₃), and C(R₃)(R₈);
R₁ is selected from the group consisting of optionally substituted alkyl, optionally substituted alkynyl, optionally substituted alkynyl, optionally substituted alkylcarbonyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₂ is selected from the group consisting of optionally substituted alkyl, optionally substituted alkynyl, optionally substituted alkynyl, optionally substituted haloalkyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical;
R₃ is selected from the group consisting of optionally substituted alkyl, optionally substituted alkynyl, optionally substituted alkynyl, optionally substituted cycloalkyl, optionally substituted heteroaryl, optionally substituted heterocyclyl, optionally substituted aryl, optionally substituted aralkyl, optionally substituted heteroaralkyl, optionally substituted (heterocyclyl)alkyl, and polyether radical; or
or R₂ and R₃, with the atoms to which they are attached form an optionally substituted cycloalkyl, an optionally substituted heteroaryl, an optionally substituted heterocyclyl, or an optionally substituted aryl;
R₄ is selected from the group consisting of hydrogen, optionally substituted alkyl, optionally substituted alkynyl, optionally substituted alkynyl, optionally substituted cycloalkyl, optionally substituted aryl, optionally substituted aralkyl, optionally substituted heteroaryl, optionally substituted heterocyclyl, halogen, optionally substituted alkoxy, optionally substituted haloalkoxy, hydroxyl, N(R₅)(R₆), and polyether radical;
R₅ and R₆ are independently selected from the group consisting of hydrogen, optionally substituted alkyl, optionally substituted acyl, optionally substituted heteroalkyl, optionally substituted aryl, optionally substituted cycloalkyl, optionally substituted heteroaryl, and optionally substituted heterocyclyl;
R₈ is selected from the group consisting of hydrogen, optionally substituted alkyl, optionally substituted alkenyl, optionally substituted alkynyl, optionally substituted cycloalkyl, optionally substituted aryl, optionally substituted aralkyl, a halogen, optionally substituted alkoxy, and hydroxyl, or can form an optionally substituted cycloalkyl, an optionally substituted heteroaryl, an optionally substituted heterocyclyl, or an optionally substituted aryl with R₃; and
R₉ is selected from the group consisting of hydrogen, optionally substituted alkyl, optionally substituted alkynyl, optionally substituted alkynyl, optionally substituted cycloalkyl, optionally substituted aryl, optionally substituted aralkyl, optionally substituted heteroaryl, optionally substituted heterocyclyl, halogen, optionally substituted alkoxy, optionally substituted haloalkoxy, hydroxyl, and N(R₅)(R₆);
provided the compound is not:

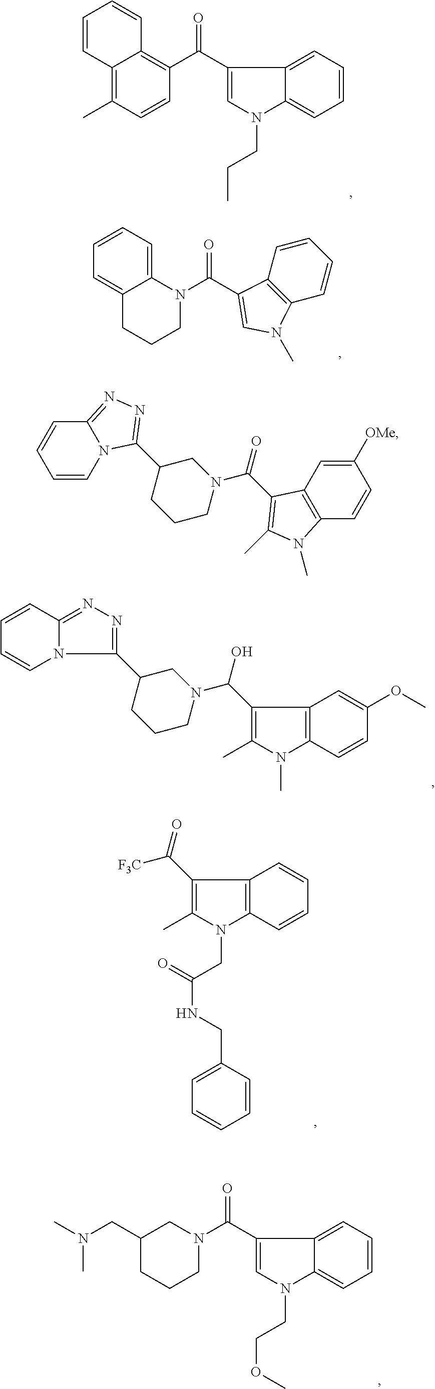

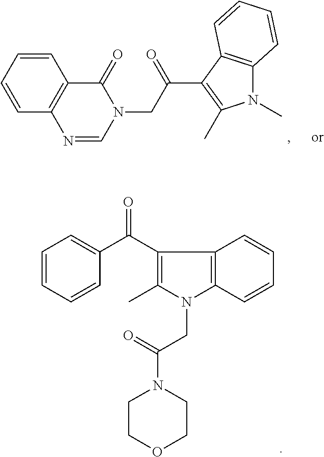 [SMILES: Cc1c(C(=O)Cn2cnc3ccccc3c2=O)c2ccccc2n1C ; Cc1c(C(=O)c2ccccc2)c2ccccc2n1CC(=O)N1CCOCC1]

20. A method of treating or inhibiting glioblastoma, a cognition disorder, schizophrenia, Alzheimer's disease and dementia, Parkinson's disease, depression, multiple sclerosis, amyotrophic lateral sclerosis (ALS), Huntington's disease, Fronto temporal dementia, parkinsonism linked to chromosome 17, prion diseases, or cancer in a subject, comprising administering to the subject an effective amount of the compound of claim 18.

21. A method of treating or inhibiting glioblastoma, a cognition disorder, schizophrenia, Alzheimer's disease and dementia, Parkinson's disease, depression, multiple sclerosis, amyotrophic lateral sclerosis (ALS), Huntington's disease, Fronto temporal dementia, parkinsonism linked to chromosome 17, prion diseases, or cancer in a subject, comprising administering to the subject an effective amount of the compound of claim 19.

22. (canceled)

23. The method according to claim 20, wherein the cancer is glioblastoma, melanoma, breast cancer, medulloblastomas, astrocytoma, or colon cancer.

24. (canceled)

25. (canceled)
